(19) United States
(12) Patent Application Publication
(10) Pub. No.: US 2015/0201943 A1
(43) Pub. Date: Jul. 23, 2015

(54) Expandable Device Useful for Anastomosis

(71) Applicant: BioStruxs, LLC, Westlake Village, CA (US)

(72) Inventors: Mai N. Brooks, Westlake Village, CA (US); James P. Watson, Los Angeles, CA (US)

(73) Assignee: BIOSTRUXS, LLC

(21) Appl. No.: 14/584,957

(22) Filed: Dec. 29, 2014

Related U.S. Application Data
(60) Provisional application No. 61/921,775, filed on Dec. 30, 2013.

(51) Int. Cl. A61B 17/11

(52) U.S. Cl. CPC A61B 17/11; A61B 2017/1103; A61B 2017/1132; A61B 2017/1107; A61B 2017/00867

(57) ABSTRACT

The present specification discloses an expandable device, methods for making such expandable devices, kits comprising one or more expandable devices, and methods and uses for implanting such expandable devices in an anastomosis procedure.

This application claims priority to U.S. Provisional Patent Application 61/921,775, filed Dec. 30, 2013, and entitled “Expandable Device Useful for Anastomosis,” which is hereby incorporated by reference in its entirety.

The circulatory system is an organ system that transports nutrients, oxygen, carbon dioxide, hormones, blood cells, and fluids to and from cells in the body to nourish it and help to fight diseases, stabilize body temperature and pH, and to maintain homeostasis. The circulatory system comprises the cardiovascular system, which carries oxygen and other nutrients to and carbon dioxide and other waste materials away from all body tissues, and the lymphatic/lymphoid system, which absorbs excess fluid from body tissues and returns it to the cardiovascular system.

The cardiovascular system comprises blood, the heart and blood vessels, which include arteries, arteriole, capillaries, venules, and veins. Blood is a fluid comprising plasma, red blood cells, white blood cells, and platelets that is circulated by the heart, which pumps oxygenated blood to the body and deoxygenated blood to the lungs. The blood vessels are the part of the circulatory system that transports blood throughout the body. Arteries and arterioles carry blood away from the heart; capillaries enable the actual exchange of material between the blood and the tissues; and veins and venules carry blood from the capillaries back toward the heart.

The lymphatic/lymphoid system functions to absorb and filter excess interstitial fluids from surrounding tissue and return the resulting lymph to the venous circulation of the cardiovascular system. The lymphatic/lymphoid system can be divided into two systems: the lymphatic system, which comprises a network of conduits composed of lymph vessels and lymph capillaries; and the lymphoid system, which comprises lymphatic organs and tissue like lymph nodes and lymphoid follicles. Lymph vessels are thin-walled, valved structures which return lymph to the cardiovascular system. Lymph enters the lymphatic system via lymph capillaries. Lymph is essentially recycled excess blood plasma after it has been filtered from the interstitial fluid and returned to the lymphatic system. Lymph vessels that carry lymph to a lymph node are called the afferent lymph vessel, and one that carries it from a lymph node is called the efferent lymph vessel, from where the lymph may travel to another lymph node, may be returned to a vein, or may travel to a larger lymph duct. Lymph ducts drain the lymph into one of the subclavian veins and thus return it to general circulation. The lymphatic system does not have a pump to propel the lymph. The movement of lymphatic fluid occurs due to contraction and relaxation of smooth muscles and valves, and due to arterial pulsation. The movement of the lymph is also facilitated by the contraction of the skeletal muscles.

Anastomosis is the connection of two structures. In reference to the medical field, it refers to connections between tubular structures such as, e.g., blood vessels, lymph vessels, loops of intestine, urogenital vessel, endocrine vessel, or respiratory vessel. Circulatory anastomoses, the joining of two circulatory vessels, can be performed to repair an injury, as a preparatory procedure for a therapeutic purpose, or to treat a circulatory-related disease. For example, blood vessels may become severed during a traumatic injury. Part of the procedure in repairing the injury would be a circulatory anastomosis that rejoins the severed vessels, thereby restoring connection of the vessels and reestablishing blood flow. As another example, individuals suffering from kidney failure may have an arteriovenous fistula created by joining an artery with a vein in the lower arm, in order to permit easier withdrawal of blood for hemodialysis. As yet another example, arteriosclerosis can be treated by creation of a coronary anastomosis during a coronary bypass surgery. Similarly, portal hypertension can be treated by surgically creating a portacaval fistula that produces an anastomosis between the hepatic portal vein and the inferior vena cava across the omental foramen. This spares the portal venous system from high pressure which can cause esophageal varices, caput medusae, and hemorrhoids.

Currently, surgical procedures used to create a circulatory anastomosis employ the use of sutures. However, the puncture of sutures through an endothelial wall of a vessel itself is a traumatic event. For example, such punctures induce a cascade of events that are biochemically mediated by trauma, create exposure of basement membrane collagen, and cause blood clots. Thus, there is a need to develop a new procedure for circulatory anastomosis that addresses and alleviates the problems associated with the use of sutures.

The present specification address the shortcomings of current surgical procedures used to create a circulatory anastomosis. The present specification discloses an expandable device used to join two vessels without the need of sutures. The design of an expandable device disclosed herein enables the device to be easier to implant during a surgical procedure and then adapt to the shape of certain blood vessels when exposed to body temperature. The expandable device disclosed herein can therefore allow for atraumatic, sutureless connection of two vessels, often without any puncture of vessel endothelium.

### SUMMARY

Aspects of the present specification provide an expandable device. An expandable device disclosed herein may comprise a tube assembly composed of an expandable metal. A tube assembly disclosed herein may comprise a tube body, a lumen, a first luminal opening and a second luminal opening, where the tube body forms the lumen and the first and second luminal openings are at each end of the tube assembly. An expandable device disclosed herein may be designed to exist in at least two different shapes each comprising a volume, the at least two different shapes comprising a first shape comprising a first volume and a first luminal diameter that occurs at a first temperature and a second shape comprising a second volume and a second luminal diameter that occurs at a second temperature.

Further aspects of the present specification provide a port in an expandable device configured for fluid communication with an interior lumen of the device and with the lumen of a vessel or other tubular structure on or in connection with which the device is employed, whereby the port of an installed expandable device according to aspects of the present specification enables, for example, the introduction of heparin or other clot dissolving drug into a vessel lumen after surgery and anastomosis is completed, beneficially avoiding the need for a follow-on surgical procedure to address the clotting off of the anastomosis.

Other aspects of the present specification provide a method of employing an expandable device disclosed herein. A method disclosed herein may comprise the steps of a) inserting one end of an expandable device disclosed herein into a first lumen of a first vessel; b) inserting the other end of an expandable device disclosed herein into a second lumen of a second vessel; and c) enlarging the expandable device. Enlargement of an expandable device disclosed herein may occur by 1) allowing the device to be warmed by the body temperature of an individual emanating from the surrounding tissue, 2) injecting a fluid into the expandable device that is sufficiently warm enough to enlarge the device; 3) using a heating device that warms the expandable device to a temperature sufficiently warm enough to enlarge the device, or any combination thereof. Likewise, a method may comprise the steps of a) inserting an end of a first lumen of a first vessel into a first end of an expandable device disclosed herein; b) inserting an end of a second lumen of a second vessel into an opposite second end of an expandable device disclosed herein; and c) contracting the expandable device. A first and second vessel may be an artery, an arteriole, a vein, a veiniole, a lymph vessel, or a lymph node. A method disclosed herein may be used to create an arterio-arterial (artery to artery) anastomosis, a veno-venous (vein to vein) anastomosis, a lympho-lymphatic anastomosis, an arterio-venous anastomosis (artery to vein), a lympho-venous anastomosis, or a lymph node to a vein anastomosis.

Other aspects of the present specification provide use of an expandable device disclosed herein to create a connection between two or more tubular structures.

Other aspects of the present specification provide a kit. A kit disclosed herein may comprise one or more expandable devices disclosed herein and may further comprise one or more containers comprising a physiological-acceptable fluid used to enlarge an expandable device, instructional material, and/or a heating device useful for warming the expandable device in order to enlarge the expandable device.

### BRIEF DESCRIPTION OF THE DRAWINGS

FIG. 1 illustrates a cross-section of a circulatory vessel diagraming the tunica intima, the tunica media, and the tunica externa.

FIGS. 2A-2Q illustrates: an end schematic view of an expandable device disclosed herein in a sealed configuration (2A); a perspective schematic view of the expandable device of FIG. 2A in a sealed configuration (2B); an end schematic view of an expandable device disclosed herein in an unsealed configuration (2C); a perspective schematic view of the expandable device of FIG. 2C in an unsealed configuration (2D); a longitudinal schematic view of a straight tube assembly (2E); a longitudinal schematic view of a reducing tube assembly (2F); a longitudinal schematic view of an angle tube assembly (2G); a longitudinal schematic view of a curve tube assembly (2H); a longitudinal schematic view of an alternative curve tube assembly (21); a longitudinal schematic view of an L-shape tube assembly (2J); a longitudinal schematic view of an alternative L-shape tube assembly (2K); a longitudinal schematic view of a tee-shape tube assembly (2L); a longitudinal schematic view of a cross-shape tube assembly (2M); a longitudinal schematic view of an alternative cross-shape tube assembly (2N); a longitudinal schematic view of an alternative reducing tube assembly (2O); a perspective schematic view of an alternative expandable device disclosed herein in an unsealed configuration (2P); and a cross-sectional schematic view of the expandable device of FIG. 2P in an unsealed configuration (2Q).

FIGS. 3A-3D illustrates: a cross-sectional schematic view of an expandable device disclosed herein in a sealed configuration in a first shape comprising a first volume inserted in a vessel (3A); a cross-sectional schematic view of the expandable device of FIG. 3A in a sealed configuration in a second shape comprising a second volume inserted in a vessel (3B); a cross-sectional schematic view of an expandable device disclosed herein in an unsealed configuration in a first shape comprising a first volume inserted in a vessel (3C); and a cross-sectional schematic view of the expandable device of FIG. 3C in an unsealed configuration in a second shape comprising a second volume inserted in a vessel (3D).

FIGS. 4A-4F illustrates: a partial perspective schematic view of an expandable device disclosed herein in a first shape comprising a first volume being implanted into a vessel during an anastomosis procedure (4A); a partial perspective schematic view of a device used to deliver a fluid attached to a port of an expandable device disclosed herein (4B); a partial perspective schematic view of an expandable device disclosed herein in a second shape comprising a second volume implanted into a vessel during an anastomosis procedure (4C); a partial perspective schematic view of an expandable device disclosed herein in a second shape where the tunica externa is pulled over the port (4D); a partial perspective schematic view of an expandable device disclosed herein in a second shape where the tunica externa is pulled over the port (4E); and a partial longitudinal sectional schematic view of an expandable device disclosed herein in a second shape where the tunica externa is pulled over the port (4F).

FIGS. 5A-5B illustrates: a partial longitudinal sectional schematic view of an implanted expandable device disclosed herein with a port protruding outwardly through a vessel and above a skin surface (5A); and a partial longitudinal sectional schematic view of an implanted expandable device disclosed herein with a port protruding outwardly through a vessel and below a skin surface (5B).

FIGS. 6A-6B illustrates: a perspective view of an expandable device disclosed herein in an unsealed configuration (6A); and an end view of the expandable device of FIG. 6A (6B).

FIGS. 7A-7B illustrates: a perspective view of an expandable device disclosed herein in an unsealed configuration (7A); and an end view of the expandable device of FIG. 7A (7B).

FIGS. 8A-8F illustrates: a partial perspective schematic view of an expandable device disclosed herein in an unsealed configuration in a second shape comprising a second volume being positioned on a vessel during an anastomosis procedure (4A); an end sectional schematic view of the expandable device of FIG. 8A in the second shape and positioned on the vessel (8B); a partial perspective schematic view of the expandable device of FIG. 8A in a first shape comprising a first volume secured on a vessel during an anastomosis procedure (8C); an end schematic view of the expandable device of FIG. 8C in the first shape and secured on the vessel (8D); a partial longitudinal sectional schematic view of an expandable device disclosed herein in a first shape where the vessel is held within the expandable device by one or more spikes (8E); and a partial longitudinal sectional schematic view of an expandable device disclosed herein in a first shape where the vessel is held within the expandable device by one or more spikes and the tunica externa is pulled onto the port (8F).

FIGS. 9A-9B illustrates: a partial longitudinal sectional schematic view of an implanted expandable device disclosed herein with a port protruding outwardly above a skin surface and inwardly through a vessel (9A); and a partial longitudinal sectional schematic view of an implanted expandable device disclosed herein with a port protruding outwardly below a skin surface and inwardly through a vessel (9B).

### DETAILED DESCRIPTION

The present specification relates generally to the anastomosis of two endothelial-lined vessels, such as, e.g., a blood vessel or a lymph vessel, though it will be appreciated that such a device may be employed in the anastomosis or joining of a variety of tubular structures, such that the anastomosis of a vessel as herein disclosed is to be understood as illustrative of aspects of the present specification and non-limiting. The expandable device disclosed herein generally comprises a self-expanding or self-contracting tube assembly formed from an expandable metal which enables the device to exist in at least two shapes of different volumes. In the exemplary embodiment wherein the expandable device is to be placed within the vessel or other tubular structure, the initial shape is typically smaller in volume and allows the expandable device to be inserted into the lumens of the cut ends of circulatory vessels. The expandable device is then enlarged to fit tightly against the internal walls of the vessels. This is accomplished in at least three different ways. First, the expandable device is made from an expandable metal which enlarges at or near body temperature (body response temperature). Second, a port may be located on the expandable device which allows for the application of warm saline to expand the sleeve to automatically fit the internal diameter of the two vessels. And third, a heating device may be used to warm the expandable device to a temperature sufficiently warm to enlarge the device. Or any combination of these three techniques or other such materials and techniques now known or later developed may be employed. In a non-limiting exemplary embodiment the expandable device indicated for placement within a vessel is made of nitinol. Expansion of the device against the interior lumen wall creates an internal channel that prevents leakage of biological fluids into the interstitial space, secures the vessels in place in a manner that joins the ends of the vessels, and prevents microvascular vasoconstriction due to the local trauma of vessel transection, such as, e.g., that which is observed in surgical procedures used to create a circulatory anastomosis. Thus, in an exemplary embodiment the expandable device disclosed herein not only accomplishes the same purpose as sutures, barbs, spikes, and the like but without any further trauma to the endothelium, but also provides additional therapeutic benefits. Alternatively, wherein the expandable device is to be placed around the outside of a vessel or other tubular structure, the initial shape is typically larger in volume and allows the vessel or lumen to be inserted within the expandable device. To accomplish this, the expandable device may be heated in any appropriate manner as above described so as to cause its expansion, only doing so before installation and use, rather than after as in the case where the device is placed inside a vessel or lumen and is to expand in order to seal and seat; rather, by heating and expanding the device pre-installation, it may then be positioned with the vessel or lumen therein and then allowed to cool to body temperature, such that the previously expanded device then contracts to seat against and seal outside of the vessel or lumen instead of inside. In a non-limiting exemplary embodiment the expandable device indicated for placement outside of a vessel is made of a material other than nitinol. It will be appreciated that a variety of geometrical and material considerations may be employed depending on the design and context, that is, whether the device is to expand at higher than body temperature and then contract in place at body temperature so as to seal about the outside of a vessel or other tubular structure with the device in its contracted configuration, versus expanding at approximately body temperature so as to seal within the inside of a vessel or other tubular structure with the device in its expanded configuration as in the first exemplary embodiment, such that any such configurations and materials of construction now known or later developed may be employed according to aspects of the present specification.

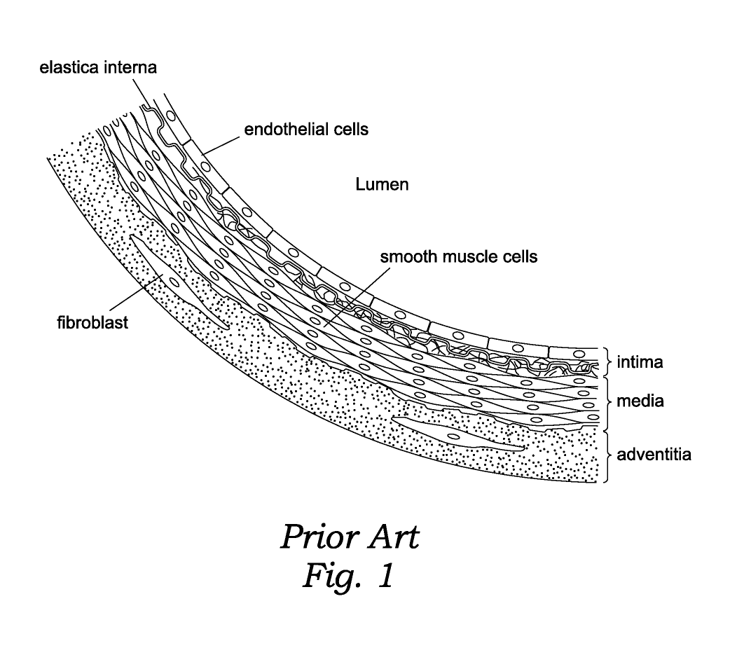

The vessels of the cardiovascular and lymphatic systems share the same general structure of three layers, the tunica intima, the tunica media, and the tunica externa or adventitia (FIG. 1). The tunica intima (“inner coat”), also known as intima, is the innermost layer of a vessel, is organized into sublayers comprising an endothelial cell sublayer, a subendothelial connective tissue sublayer, and an internal elastic membrane (also called the internal elastic lamina or elastic interna). The endothelial cell sublayer comprises endothelial cells attached to a polysaccharide intercellular matrix. This sublayer is in direct contact with the blood or lymph and functions to mechanically transport these fluids. The endothelial cell sublayer is surrounded by the subendothelial connective tissue sublayer, which, in turn, is interlaced with the internal elastic membrane. This outer sublayer comprises a network of circularly arranged elastic fibers, having principally a longitudinal direction, and is lacking in veins.

The tunica media (“middle coat”), also known as media, is the middle layer of a vessel, and thus lies between the tunica intima on the inside and the tunica externa on the outside. This middle layer comprises circularly arranged smooth muscle cells and elastic fibers. The contraction and relaxation of the muscle cells controls the luminal diameter (caliber) of the vessel, which facilitates the transportation of fluids.

The tunica externa (“outer coat”), also known as externa, tunica adventitia or adventitia, is the outermost layer of a vessel, and surrounds the tunica media. The tunica externa is a fibrous connective tissue comprising collagen and enables the vessels to withstand internal fluid pressure as well as serves to anchor the vessel to nearby tissue, giving it stability. In arteries, this outer layer further includes an elastic or fenestrated layer called an external elastic membrane (also known as external elastic lamina or elastic externa). The tunica externa also contains nerves that supply the vessel as well as nutrient capillaries (vasa vasorum) in the larger blood vessels.

Both blood and lymph capillaries are very thin vessels typically consisting of little more than a single endothelial cell layer and occasional connective tissue, and lacking tunica media and tunica externa layers. Blood capillaries are the sites where oxygen and other nutrients are transferred from the arterial bloodstream to the surrounding tissue in the body, and where carbon dioxide and other waste materials and fluids are collected for return to the veins. Lymph capillaries have a unique structure of overlapping endothelial cells that permits interstitial fluid to flow into the lymphatic system to create lymph, but not out into the surrounding tissue. Lymph capillaries are slightly larger than their counterpart capillaries of the cardiovascular system.

Aspects of the present specification disclose, in part, an expandable device. The disclosed expandable device is useful for an end-to-end anastomosis of two vessels without the need of sutures. In one embodiment, a vessel is a circulatory vessel. In aspects of this embodiment, a circulatory vessel is a blood vessel or a lymph vessel. Vessels that can be joined using an expandable device disclosed herein, include, without limitation, an arterio-arterial anastomosis (artery to artery or arteriole to arteriole), a veno-venous anastomosis (vein to vein or veiniole to veiniole), a lympho-lymphatic anastomosis (lymphatic vessel to lymphatic vessel), an arterio-venous anastomosis (artery to vein or arteriole to veiniole), a lympho-venous anastomosis (lymphatic vessel to vein), or a lymph node to a vein.

Typically, an expandable device disclosed herein is a tube assembly. In such an assembly, a sheet of an expandable metal as disclosed herein is shaped into a tube comprising a tube body having a length and forming a lumen. A tube assembly disclosed herein is open at both ends, which may be designated as a first luminal opening and a second luminal opening. A tube assembly disclosed herein may be designed to conform to the junction the device is being employed to connect. In aspects of this embodiment, a tube assembly disclosed herein may be designed as a straight tube, an angled tube, a curved tube, an L-shaped tube, a tee-shaped tube, a cross-shaped tube, or a reducer tube where a portion of the tube assembly is one size and another portion of the tube assembly is of a larger size. Shape designs of tube assemblies disclosed herein having angles may have these angles configured as shape angles or curved angles. In one embodiment, a tube assembly disclosed herein is cylindrical in shape and comprises a cylindrical body forming a lumen where the cylindrical body has a first luminal opening and a second luminal opening.

An expandable device comprising a tube assembly disclosed herein may be designed in a sealed configuration or in an unsealed configuration. In a sealed configuration, the integrity of a tube body is intact and expansion occurs through the elasticity possessed by the expandable metal or other material. In an unsealed configuration, a tube body is split and an overlap region is formed by a sheet of expandable metal, where one longitudinal or lengthwise edge of the expandable metal covers or overlaps the other such edge of the expandable metal. In an unsealed configuration, expansion occurs through the movement of the sheet of expandable metal as is uncoils in lieu of, or in addition to, through the elasticity possessed by the expandable metal.

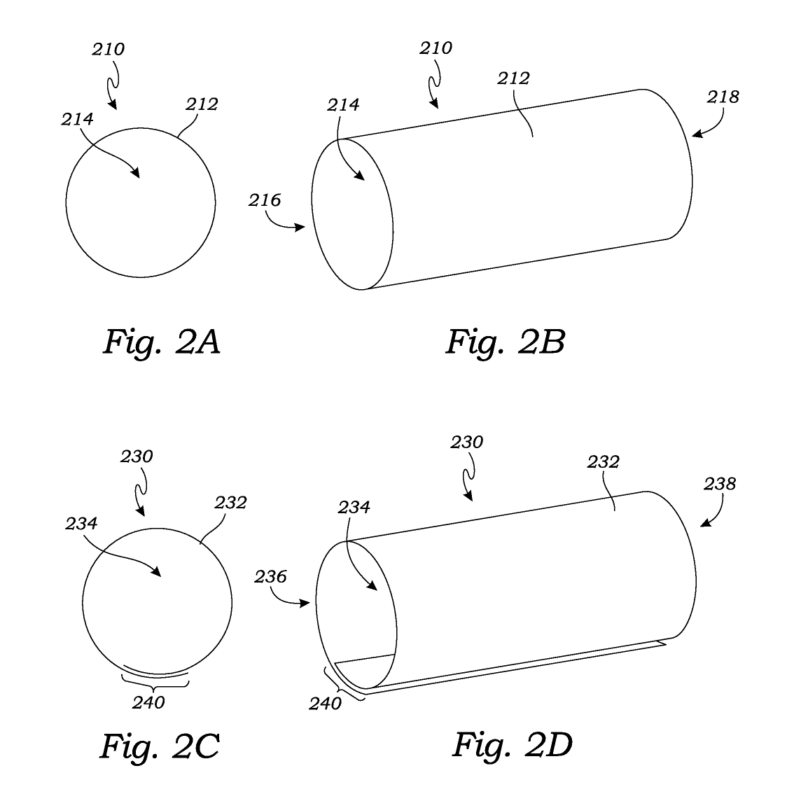

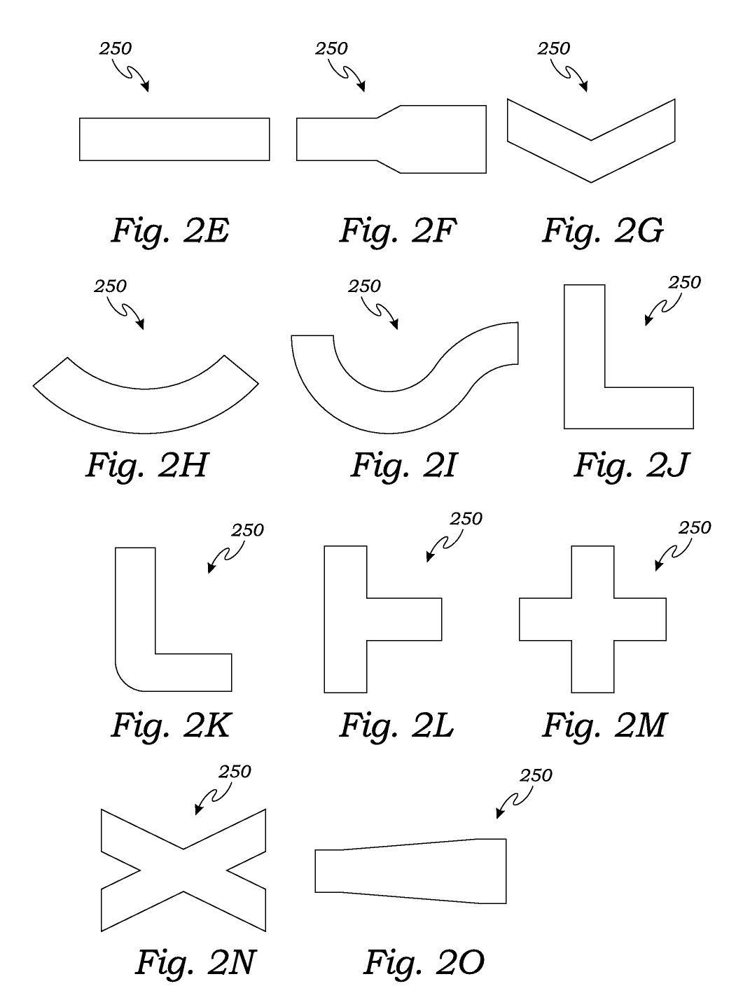

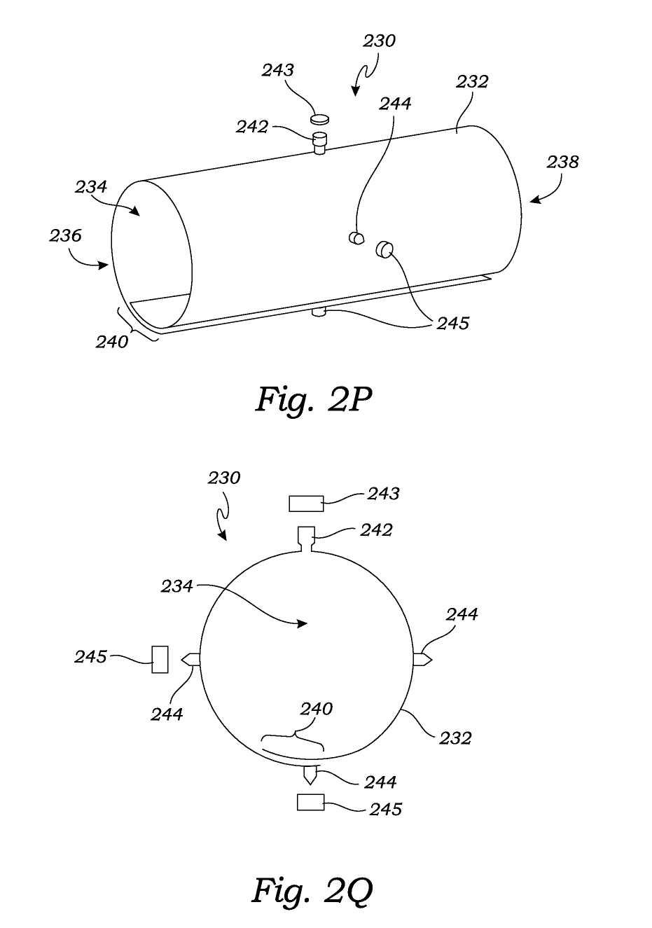

FIG. 2 illustrates one embodiment of an expandable device comprising a tube assembly disclosed herein designed in a sealed configuration. FIG. 2A shows a cross-sectional or end schematic view of expandable device 210, including intact tube body 212 and lumen 214. FIG. 2B shows a perspective schematic view of expandable device 210, again including intact tube body 212 and lumen 214, as well as first luminal opening 216 and second luminal opening 218.

FIG. 2 further illustrates one embodiment of an expandable device comprising a tube assembly disclosed herein designed in an unsealed configuration. FIG. 2C shows a cross-sectional or end schematic view of expandable device 230, including split tube body 232, overlap region 240, and lumen 234. FIG. 2D shows a perspective schematic view of expandable device 230, including split tube body 232, overlap region 240, lumen 234, first luminal opening 236, and second luminal opening 238. FIG. 2P shows a perspective schematic view of expandable device 230, including split tube body 232, overlap region 240, lumen 234, first luminal opening 236, second luminal opening 238, port 242, port cap 243, spike 244, and spike caps 245. FIG. 2Q shows an end schematic view of expandable device 230, again including split tube body 232, overlap region 240, lumen 234, port 242, port cap 243, spikes 244, and spike caps 245.

FIG. 2 further illustrates some exemplary embodiments of shape designs of tube assemblies disclosed herein as longitudinal schematic views thereof: FIG. 2E of a straight tube assembly; FIG. 2F of a first reducing tube assembly; FIG. 2G of an angle tube assembly; FIG. 2H of a first curve tube assembly; FIG. 2I of a second curve tube assembly; FIG. 2J of a first L-shape tube assembly; FIG. 2K of a second L-shape tube assembly; FIG. 2L of a tee-shape tube assembly; FIG. 2M of a first cross-shape tube assembly; FIG. 2N of a second cross-shape tube assembly; and FIG. 2O of a second reducing tube assembly.

An expandable device disclosed herein has a body length suitable to facilitate joining of at least two vessels or other tubular structures. In aspects of this embodiment, an expandable device disclosed herein has a body length of, e.g., about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19, or about 20 mm. In other aspects of this embodiment, an expandable device disclosed herein has a body length of, e.g., at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, or at least 15 mm, at least 16 mm, at least 17 mm, at least 18 mm, at least 19 mm, or at least 20 mm. In yet other aspects of this embodiment, an expandable device disclosed herein has a body length of, e.g., at most 2 mm, at most 3 mm, at most 4 mm, at most 5 mm, at most 6 mm, at most 7 mm, at most 8 mm, at most 9 mm, at most 10 mm, at most 11 mm, at most 12 mm, at most 13 mm, at most 14 mm, at most 15 mm, at most 16 mm, at most 17 mm, at most 18 mm, at most 19 mm, or at most 20 mm.

In still other aspects of this embodiment, an expandable device disclosed herein has a body length of, e.g., about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 2 mm to about 11 mm, about 2 mm to about 12 mm, about 2 mm to about 13 mm, about 2 mm to about 14 mm, about 2 mm to about 15 mm, about 2 mm to about 16 mm, about 2 mm to about 17 mm, about 2 mm to about 18 mm, about 2 mm to about 19 mm, about 2 mm to about 20 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 3 mm to about 11 mm, about 3 mm to about 12 mm, about 3 mm to about 13 mm, about 3 mm to about 14 mm, about 3 mm to about 15 mm, about 3 mm to about 16 mm, about 3 mm to about 17 mm, about 3 mm to about 18 mm, about 2 mm to about 19 mm, about 2 mm to about 20 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 4 mm to about 11 mm, about 4 mm to about 12 mm, about 4 mm to about 13 mm, about 4 mm to about 14 mm, about 4 mm to about 15 mm, about 4 mm to about 16 mm, about 4 mm to about 17 mm, about 4 mm to about 18 mm, about 4 mm to about 19 mm, about 4 mm to about 20 mm, about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 5 mm to about 13 mm, about 5 mm to about 14 mm, about 5 mm to about 15 mm, about 5 mm to about 16 mm, about 5 mm to about 17 mm, about 5 mm to about 18 mm, about 5 mm to about 19 mm, about 5 mm to about 20 mm, about 6 mm to about 7 mm, about 6 mm to about 8 mm, about 6 mm to about 9 mm, about 6 mm to about 10 mm, about 6 mm to about 11 mm, about 6 mm to about 12 mm, about 6 mm to about 13 mm, about 6 mm to about 14 mm, about 6 mm to about 15 mm, about 6 mm to about 16 mm, about 6 mm to about 17 mm, about 6 mm to about 18 mm, about 6 mm to about 19 mm, about 6 mm to about 20 mm, about 7 mm to about 8 mm, about 7 mm to about 9 mm, about 7 mm to about 10 mm, about 7 mm to about 11 mm, about 7 mm to about 12 mm, about 7 mm to about 13 mm, about 7 mm to about 14 mm, about 7 mm to about 15 mm, about 7 mm to about 16 mm, about 7 mm to about 17 mm, about 7 mm to about 18 mm, about 7 mm to about 19 mm, about 7 mm to about 20 mm, about 8 mm to about 9 mm, about 8 mm to about 10 mm, about 8 mm to about 11 mm, about 8 mm to about 12 mm, about 8 mm to about 13 mm, about 8 mm to about 14 mm, about 8 mm to about 15 mm, about 8 mm to about 16 mm, about 8 mm to about 17 mm, about 8 mm to about 18 mm, about 8 mm to about 19 mm, about 8 mm to about 20 mm, about 9 mm to about 10 mm, about 9 mm to about 11 mm, about 9 mm to about 12 mm, about 9 mm to about 13 mm, about 9 mm to about 14 mm, about 9 mm to about 15 mm, about 9 mm to about 16 mm, about 9 mm to about 17 mm, about 9 mm to about 18 mm, about 9 mm to about 19 mm, about 9 mm to about 20 mm, about 10 mm to about 11 mm, about 10 mm to about 12 mm, about 10 mm to about 13 mm, about 10 mm to about 14 mm, about 10 mm to about 15 mm, about 10 mm to about 16 mm, about 10 mm to about 17 mm, about 10 mm to about 18 mm, about 10 mm to about 19 mm, about 10 mm to about 20 mm, about 11 mm to about 12 mm, about 11 mm to about 13 mm, about 11 mm to about 14 mm, about 11 mm to about 15 mm, about 11 mm to about 16 mm, about 11 mm to about 17 mm, about 11 mm to about 18 mm, about 11 mm to about 19 mm, about 11 mm to about 20 mm, about 12 mm to about 13 mm, about 12 mm to about 14 mm, about 12 mm to about 15 mm, about 12 mm to about 16 mm, about 12 mm to about 17 mm, about 12 mm to about 18 mm, about 12 mm to about 19 mm, about 12 mm to about 20 mm, about 13 mm to about 14 mm, about 13 mm to about 15 mm, about 13 mm to about 16 mm, about 13 mm to about 17 mm, about 13 mm to about 18 mm, about 13 mm to about 19 mm, about 13 mm to about 20 mm, about 14 mm to about 15 mm, about 14 mm to about 16 mm, about 14 mm to about 17 mm, about 14 mm to about 18 mm, about 14 mm to about 19 mm, about 14 mm to about 20 mm, about 15 mm to about 16 mm, about 15 mm to about 17 mm, about 15 mm to about 18 mm, about 15 mm to about 19 mm, about 15 mm to about 20 mm, about 16 mm to about 17 mm, about 16 mm to about 18 mm, about 16 mm to about 19 mm, about 16 mm to about 20 mm, about 17 mm to about 18 mm, about 17 mm to about 19 mm, about 17 mm to about 20 mm, about 18 mm to about 19 mm, about 18 mm to about 20 mm, or about 19 mm to about 20 mm.

In aspects of this embodiment, an expandable device disclosed herein has a body length of, e.g., about 0.1 cm, about 0.2 cm, about 0.3 cm, about 0.4 cm, about 0.5 cm, about 0.6 cm, about 0.7 cm, about 0.8 cm, about 0.9 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, about 2.5 cm, about 2.6 cm, about 2.7 cm, about 2.8 cm, about 2.9 cm, about 3.0 cm, about 3.1 cm, about 3.2 cm, about 3.3 cm, about 3.4 cm, about 3.5 cm, about 3.6 cm, about 3.7 cm, about 3.8 cm, about 3.9 cm, or about 4.0 cm. In other aspects of this embodiment, an expandable device disclosed herein has a body length of, e.g., at least 0.1 cm, at least 0.2 cm, at least 0.3 cm, at least 0.4 cm, at least 0.5 cm, at least 0.6 cm, at least 0.7 cm, at least 0.8 cm, at least 0.9 cm, at least 1.0 cm, at least 1.1 cm, at least 1.2 cm, at least 1.3 cm, at least 1.4 cm, at least 1.5 cm, at least 1.6 cm, at least 1.7 cm, at least 1.8 cm, at least 1.9 cm, at least 2.0 cm, at least 2.1 cm, at least 2.2 cm, at least 2.3 cm, at least 2.4 cm, at least 2.5 cm, at least 2.6 cm, at least 2.7 cm, at least 2.8 cm, at least 2.9 cm, at least 3.0 cm, at least 3.1 cm, at least 3.2 cm, at least 3.3 cm, at least 3.4 cm, at least 3.5 cm, at least 3.6 cm, at least 3.7 cm, at least 3.8 cm, at least 3.9 cm, or at least 4.0 cm. In yet other aspects of this embodiment, an expandable device disclosed herein has a body length of, e.g., at most 0.1 cm, at most 0.2 cm, at most 0.3 cm, at most 0.4 cm, at most 0.5 cm, at most 0.6 cm, at most 0.7 cm, at most 0.8 cm, at most 0.9 cm, at most 1.0 cm, at most 1.1 cm, at most 1.2 cm, at most 1.3 cm, at most 1.4 cm, at most 1.5 cm, at most 1.6 cm, at most 1.7 cm, at most 1.8 cm, at most 1.9 cm, at most 2.0 cm, at most 2.1 cm, at most 2.2 cm, at most 2.3 cm, at most 2.4 cm, at most 2.5 cm, at most 2.6 cm, at most 2.7 cm, at most 2.8 cm, at most 2.9 cm, at most 3.0 cm, at most 3.1 cm, at most 3.2 cm, at most 3.3 cm, at most 3.4 cm, at most 3.5 cm, at most 3.6 cm, at most 3.7 cm, at most 3.8 cm, at most 3.9 cm, or at most 4.0 cm.

In still other aspects of this embodiment, an expandable device disclosed herein has a body length of, e.g., about 0.1 cm to about 0.5 cm, about 0.1 cm to about 0.75 cm, about 0.1 cm to about 1.0 cm, about 0.1 cm to about 1.25 cm, about 0.1 cm to about 1.5 cm, about 0.1 cm to about 1.75 cm, about 0.1 cm to about 2.0 cm, about 0.1 cm to about 2.25 cm, about 0.1 cm to about 2.5 cm, about 0.1 cm to about 2.75 cm, about 0.1 cm to about 3.0 cm, about 0.1 cm to about 3.25 cm, about 0.1 cm to about 3.5 cm, about 0.1 cm to about 3.75 cm, about 0.1 cm to about 4.0 cm, about 0.25 cm to about 0.5 cm, about 0.25 cm to about 0.75 cm, about 0.25 cm to about 1.0 cm, about 0.25 cm to about 1.25 cm, about 0.25 cm to about 1.5 cm, about 0.25 cm to about 1.75 cm, about 0.25 cm to about 2.0 cm, about 0.25 cm to about 2.25 cm, about 0.25 cm to about 2.5 cm, about 0.25 cm to about 2.75 cm, about 0.25 cm to about 3.0 cm, about 0.25 cm to about 3.25 cm, about 0.25 cm to about 3.5 cm, about 0.25 cm to about 3.75 cm, about 0.25 cm to about 4.0 cm, about 0.5 cm to about 0.75 cm, about 0.5 cm to about 1.0 cm, about 0.5 cm to about 1.25 cm, about 0.5 cm to about 1.5 cm, about 0.5 cm to about 1.75 cm, about 0.5 cm to about 2.0 cm, about 0.5 cm to about 2.25 cm, about 0.5 cm to about 2.5 cm, about 0.5 cm to about 2.75 cm, about 0.5 cm to about 3.0 cm, about 0.5 cm to about 3.25 cm, about 0.5 cm to about 3.5 cm, about 0.5 cm to about 3.75 cm, about 0.5 cm to about 4.0 cm, about 0.75 cm to about 1.0 cm, about 0.75 cm to about 1.25 cm, about 0.75 cm to about 1.5 cm, about 0.75 cm to about 1.75 cm, about 0.75 cm to about 2.0 cm, about 0.75 cm to about 2.25 cm, about 0.75 cm to about 2.5 cm, about 0.75 cm to about 2.75 cm, about 0.75 cm to about 3.0 cm, about 0.75 cm to about 3.25 cm, about 0.75 cm to about 3.5 cm, about 0.75 cm to about 3.75 cm, about 0.75 cm to about 4.0 cm, about 1.0 cm to about 1.25 cm, about 1.0 cm to about 1.5 cm, about 1.0 cm to about 1.75 cm, about 1.0 cm to about 2.0 cm, about 1.0 cm to about 2.25 cm, about 1.0 cm to about 2.5 cm, about 1.0 cm to about 2.75 cm, about 1.0 cm to about 3.0 cm, about 1.0 cm to about 3.25 cm, about 1.0 cm to about 3.5 cm, about 1.0 cm to about 3.75 cm, about 1.0 cm to about 4.0 cm, about 1.5 cm to about 2.0 cm, about 1.5 cm to about 2.25 cm, about 1.5 cm to about 2.5 cm, about 1.5 cm to about 2.75 cm, about 1.5 cm to about 3.0 cm, about 1.5 cm to about 3.25 cm, about 1.5 cm to about 3.5 cm, about 1.5 cm to about 3.75 cm, about 1.5 cm to about 4.0 cm, about 2.0 cm to about 2.25 cm, about 2.0 cm to about 2.5 cm, about 2.0 cm to about 2.75 cm, or about 2.0 cm to about 3.0 cm, about 2.0 cm to about 3.25 cm, about 2.0 cm to about 3.5 cm, about 2.0 cm to about 3.75 cm, about 2.0 cm to about 4.0 cm, about 2.5 cm to about 2.75 cm, or about 2.5 cm to about 3.0 cm, about 2.5 cm to about 3.25 cm, about 2.5 cm to about 3.5 cm, about 2.5 cm to about 3.75 cm, about 2.5 cm to about 4.0 cm, about 3.0 cm to about 3.25 cm, about 3.0 cm to about 3.5 cm, about 3.0 cm to about 3.75 cm, about 3.0 cm to about 4.0 cm, about 3.5 cm to about 3.75 cm, or about 3.5 cm to about 4.0 cm.

In aspects of this embodiment, an expandable device disclosed herein has a body length of, e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19, about 20 cm, about 21 cm, about 22 cm, about 23 cm, about 24 cm, about 25 cm, about 26 cm, about 27 cm, about 28 cm, about 29 cm, or about 30 cm. In other aspects of this embodiment, an expandable device disclosed herein has a body length of, e.g., at least 1 cm, at least 2 cm, at least 3 cm, at least 4 cm, at least 5 cm, at least 6 cm, at least 7 cm, at least 8 cm, at least 9 cm, at least 10 cm, at least 11 cm, at least 12 cm, at least 13 cm, at least 14 cm, or at least 15 cm, at least 16 cm, at least 17 cm, at least 18 cm, at least 19 cm, at least 20 cm, at least 21 cm, at least 22 cm, at least 23 cm, at least 24 cm, at least 25 cm, at least 26 cm, at least 27 cm, at least 28 cm, at least 29 cm, or at least 30 cm. In yet other aspects of this embodiment, an expandable device disclosed herein has a body length of, e.g., at most 1 cm, at most 2 cm, at most 3 cm, at most 4 cm, at most 5 cm, at most 6 cm, at most 7 cm, at most 8 cm, at most 9 cm, at most 10 cm, at most 11 cm, at most 12 cm, at most 13 cm, at most 14 cm, at most 15 cm, at most 16 cm, at most 17 cm, at most 18 cm, at most 19 cm, at most 20 cm, at most 21 cm, at most 22 cm, at most 23 cm, at most 24 cm, at most 25 cm, at most 26 cm, at most 27 cm, at most 28 cm, at most 29 cm, or at most 30 cm.

In still other aspects of this embodiment, an expandable device disclosed herein has a body length of, e.g., about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 2 cm to about 11 cm, about 2 cm to about 12 cm, about 2 cm to about 13 cm, about 2 cm to about 14 cm, about 2 cm to about 15 cm, about 2 cm to about 16 cm, about 2 cm to about 17 cm, about 2 cm to about 18 cm, about 2 cm to about 19 cm, about 2 cm to about 20 cm, about 2 cm to about 21 cm, about 2 cm to about 22 cm, about 2 cm to about 23 cm, about 2 cm to about 24 cm, about 2 cm to about 25 cm, about 2 cm to about 26 cm, about 2 cm to about 27 cm, about 2 cm to about 28 cm, about 2 cm to about 29 cm, about 2 cm to about 30 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 3 cm to about 11 cm, about 3 cm to about 12 cm, about 3 cm to about 13 cm, about 3 cm to about 14 cm, about 3 cm to about 15 cm, about 3 cm to about 16 cm, about 3 cm to about 17 cm, about 3 cm to about 18 cm, about 3 cm to about 19 cm, about 3 cm to about 20 cm, about 3 cm to about 21 cm, about 3 cm to about 22 cm, about 3 cm to about 23 cm, about 3 cm to about 24 cm, about 3 cm to about 25 cm, about 3 cm to about 26 cm, about 3 cm to about 27 cm, about 3 cm to about 28 cm, about 3 cm to about 29 cm, about 3 cm to about 30 cm, about 4 cm to about 5 cm, about 4 cm to about 6 cm, about 4 cm to about 7 cm, about 4 cm to about 8 cm, about 4 cm to about 9 cm, about 4 cm to about 10 cm, about 4 cm to about 11 cm, about 4 cm to about 12 cm, about 4 cm to about 13 cm, about 4 cm to about 14 cm, about 4 cm to about 15 cm, about 4 cm to about 16 cm, about 4 cm to about 17 cm, about 4 cm to about 18 cm, about 4 cm to about 19 cm, about 4 cm to about 20 cm, about 4 cm to about 21 cm, about 4 cm to about 22 cm, about 4 cm to about 23 cm, about 4 cm to about 24 cm, about 4 cm to about 25 cm, about 4 cm to about 26 cm, about 4 cm to about 27 cm, about 4 cm to about 28 cm, about 4 cm to about 29 cm, about 4 cm to about 30 cm, about 5 cm to about 6 cm, about 5 cm to about 7 cm, about 5 cm to about 8 cm, about 5 cm to about 9 cm, about 5 cm to about 10 cm, about 5 cm to about 11 cm, about 5 cm to about 12 cm, about 5 cm to about 13 cm, about 5 cm to about 14 cm, about 5 cm to about 15 cm, about 5 cm to about 16 cm, about 5 cm to about 17 cm, about 5 cm to about 18 cm, about 5 cm to about 19 cm, about 5 cm to about 20 cm, about 5 cm to about 21 cm, about 5 cm to about 22 cm, about 5 cm to about 23 cm, about 5 cm to about 24 cm, about 5 cm to about 25 cm, about 5 cm to about 26 cm, about 5 cm to about 27 cm, about 5 cm to about 28 cm, about 5 cm to about 29 cm, about 5 cm to about 30 cm, about 6 cm to about 7 cm, about 6 cm to about 8 cm, about 6 cm to about 9 cm, about 6 cm to about 10 cm, about 6 cm to about 11 cm, about 6 cm to about 12 cm, about 6 cm to about 13 cm, about 6 cm to about 14 cm, about 6 cm to about 15 cm, about 6 cm to about 16 cm, about 6 cm to about 17 cm, about 6 cm to about 18 cm, about 6 cm to about 19 cm, about 6 cm to about 20 cm, about 6 cm to about 21 cm, about 6 cm to about 22 cm, about 6 cm to about 23 cm, about 6 cm to about 24 cm, about 6 cm to about 25 cm, about 6 cm to about 26 cm, about 6 cm to about 27 cm, about 6 cm to about 28 cm, about 6 cm to about 29 cm, about 6 cm to about 30 cm, about 7 cm to about 8 cm, about 7 cm to about 9 cm, about 7 cm to about 10 cm, about 7 cm to about 11 cm, about 7 cm to about 12 cm, about 7 cm to about 13 cm, about 7 cm to about 14 cm, about 7 cm to about 15 cm, about 7 cm to about 16 cm, about 7 cm to about 17 cm, about 7 cm to about 18 cm, about 7 cm to about 19 cm, about 7 cm to about 20 cm, about 7 cm to about 21 cm, about 7 cm to about 22 cm, about 7 cm to about 23 cm, about 7 cm to about 24 cm, about 7 cm to about 25 cm, about 7 cm to about 26 cm, about 7 cm to about 27 cm, about 7 cm to about 28 cm, about 7 cm to about 29 cm, about 7 cm to about 30 cm, about 8 cm to about 9 cm, about 8 cm to about 10 cm, about 8 cm to about 11 cm, about 8 cm to about 12 cm, about 8 cm to about 13 cm, about 8 cm to about 14 cm, about 8 cm to about 15 cm, about 8 cm to about 16 cm, about 8 cm to about 17 cm, about 8 cm to about 18 cm, about 8 cm to about 19 cm, about 8 cm to about 20 cm, about 8 cm to about 21 cm, about 8 cm to about 22 cm, about 8 cm to about 23 cm, about 8 cm to about 24 cm, about 8 cm to about 25 cm, about 8 cm to about 26 cm, about 8 cm to about 27 cm, about 8 cm to about 28 cm, about 8 cm to about 29 cm, about 8 cm to about 30 cm, about 9 cm to about 10 cm, about 9 cm to about 11 cm, about 9 cm to about 12 cm, about 9 cm to about 13 cm, about 9 cm to about 14 cm, about 9 cm to about 15 cm, about 9 cm to about 16 cm, about 9 cm to about 17 cm, about 9 cm to about 18 cm, about 9 cm to about 19 cm, about 9 cm to about 20 cm, about 9 cm to about 21 cm, about 9 cm to about 22 cm, about 9 cm to about 23 cm, about 9 cm to about 24 cm, about 9 cm to about 25 cm, about 9 cm to about 26 cm, about 9 cm to about 27 cm, about 9 cm to about 28 cm, about 9 cm to about 29 cm, about 9 cm to about 30 cm, about 10 cm to about 11 cm, about 10 cm to about 12 cm, about 10 cm to about 13 cm, about 10 cm to about 14 cm, about 10 cm to about 15 cm, about 10 cm to about 16 cm, about 10 cm to about 17 cm, about 10 cm to about 18 cm, about 10 cm to about 19 cm, about 10 cm to about 20 cm, about 10 cm to about 21 cm, about 10 cm to about 22 cm, about 10 cm to about 23 cm, about 10 cm to about 24 cm, about 10 cm to about 25 cm, about 10 cm to about 26 cm, about 10 cm to about 27 cm, about 10 cm to about 28 cm, about 10 cm to about 29 cm, about 10 cm to about 30 cm, about 11 cm to about 12 cm, about 11 cm to about 13 cm, about 11 cm to about 14 cm, about 11 cm to about 15 cm, about 11 cm to about 16 cm, about 11 cm to about 17 cm, about 11 cm to about 18 cm, about 11 cm to about 19 cm, about 11 cm to about 20 cm, about 11 cm to about 21 cm, about 11 cm to about 22 cm, about 11 cm to about 23 cm, about 11 cm to about 24 cm, about 11 cm to about 25 cm, about 11 cm to about 26 cm, about 11 cm to about 27 cm, about 11 cm to about 28 cm, about 11 cm to about 29 cm, about 11 cm to about 30 cm, about 12 cm to about 13 cm, about 12 cm to about 14 cm, about 12 cm to about 15 cm, about 12 cm to about 16 cm, about 12 cm to about 17 cm, about 12 cm to about 18 cm, about 12 cm to about 19 cm, about 12 cm to about 20 cm, about 12 cm to about 21 cm, about 12 cm to about 22 cm, about 12 cm to about 23 cm, about 12 cm to about 24 cm, about 12 cm to about 25 cm, about 12 cm to about 26 cm, about 12 cm to about 27 cm, about 12 cm to about 28 cm, about 12 cm to about 29 cm, about 12 cm to about 30 cm, about 13 cm to about 14 cm, about 13 cm to about 15 cm, about 13 cm to about 16 cm, about 13 cm to about 17 cm, about 13 cm to about 18 cm, about 13 cm to about 19 cm, about 13 cm to about 20 cm, about 13 cm to about 21 cm, about 13 cm to about 22 cm, about 13 cm to about 23 cm, about 13 cm to about 24 cm, about 13 cm to about 25 cm, about 13 cm to about 26 cm, about 13 cm to about 27 cm, about 13 cm to about 28 cm, about 13 cm to about 29 cm, about 13 cm to about 30 cm, about 14 cm to about 15 cm, about 14 cm to about 16 cm, about 14 cm to about 17 cm, about 14 cm to about 18 cm, about 14 cm to about 19 cm, about 14 cm to about 20 cm, about 14 cm to about 21 cm, about 14 cm to about 22 cm, about 14 cm to about 23 cm, about 14 cm to about 24 cm, about 14 cm to about 25 cm, about 14 cm to about 26 cm, about 14 cm to about 27 cm, about 14 cm to about 28 cm, about 14 cm to about 29 cm, about 14 cm to about 30 cm, about 15 cm to about 16 cm, about 15 cm to about 17 cm, about 15 cm to about 18 cm, about 15 cm to about 19 cm, about 15 cm to about 20 cm, about 15 cm to about 21 cm, about 15 cm to about 22 cm, about 15 cm to about 23 cm, about 15 cm to about 24 cm, about 15 cm to about 25 cm, about 15 cm to about 26 cm, about 15 cm to about 27 cm, about 15 cm to about 28 cm, about 15 cm to about 29 cm, about 15 cm to about 30 cm, about 16 cm to about 17 cm, about 16 cm to about 18 cm, about 16 cm to about 19 cm, about 16 cm to about 20 cm, about 16 cm to about 21 cm, about 16 cm to about 22 cm, about 16 cm to about 23 cm, about 16 cm to about 24 cm, about 16 cm to about 25 cm, about 16 cm to about 26 cm, about 16 cm to about 27 cm, about 16 cm to about 28 cm, about 16 cm to about 29 cm, about 16 cm to about 30 cm, about 17 cm to about 18 cm, about 17 cm to about 19 cm, about 17 cm to about 20 cm, about 17 cm to about 21 cm, about 17 cm to about 22 cm, about 17 cm to about 23 cm, about 17 cm to about 24 cm, about 17 cm to about 25 cm, about 17 cm to about 26 cm, about 17 cm to about 27 cm, about 17 cm to about 28 cm, about 17 cm to about 29 cm, about 17 cm to about 30 cm, about 18 cm to about 19 cm, about 18 cm to about 20 cm, about 18 cm to about 21 cm, about 18 cm to about 22 cm, about 18 cm to about 23 cm, about 18 cm to about 24 cm, about 18 cm to about 25 cm, about 18 cm to about 26 cm, about 18 cm to about 27 cm, about 18 cm to about 28 cm, about 18 cm to about 29 cm, about 18 cm to about 30 cm, about 19 cm to about 20 cm, about 19 cm to about 21 cm, about 19 cm to about 22 cm, about 19 cm to about 23 cm, about 19 cm to about 24 cm, about 19 cm to about 25 cm, about 19 cm to about 26 cm, about 19 cm to about 27 cm, about 19 cm to about 28 cm, about 19 cm to about 29 cm, about 19 cm to about 30 cm, about 20 cm to about 21 cm, about 20 cm to about 22 cm, about 20 cm to about 23 cm, about 20 cm to about 24 cm, about 20 cm to about 25 cm, about 20 cm to about 26 cm, about 20 cm to about 27 cm, about 20 cm to about 28 cm, about 20 cm to about 29 cm, about 20 cm to about 30 cm, about 21 cm to about 22 cm, about 21 cm to about 23 cm, about 21 cm to about 24 cm, about 21 cm to about 25 cm, about 21 cm to about 26 cm, about 21 cm to about 27 cm, about 21 cm to about 28 cm, about 21 cm to about 29 cm, about 21 cm to about 30 cm, about 22 cm to about 23 cm, about 22 cm to about 24 cm, about 22 cm to about 25 cm, about 22 cm to about 26 cm, about 22 cm to about 27 cm, about 22 cm to about 28 cm, about 22 cm to about 29 cm, about 22 cm to about 30 cm, about 23 cm to about 24 cm, about 23 cm to about 25 cm, about 23 cm to about 26 cm, about 23 cm to about 27 cm, about 23 cm to about 28 cm, about 23 cm to about 29 cm, about 23 cm to about 30 cm, about 24 cm to about 25 cm, about 24 cm to about 26 cm, about 24 cm to about 27 cm, about 24 cm to about 28 cm, about 24 cm to about 29 cm, about 24 cm to about 30 cm, about 25 cm to about 26 cm, about 25 cm to about 27 cm, about 25 cm to about 28 cm, about 25 cm to about 29 cm, about 25 cm to about 30 cm, about 26 cm to about 27 cm, about 26 cm to about 28 cm, about 26 cm to about 29 cm, about 26 cm to about 30 cm, about 27 cm to about 28 cm, about 27 cm to about 29 cm, about 27 cm to about 30 cm, about 28 cm to about 29 cm, about 28 cm to about 30 cm, or about 28 cm to about 30 cm.

An expandable device disclosed herein may also comprise an overlap region. Typically, an expandable device disclosed herein designed in an unsealed configuration will comprise an overlap region. An overlap region is of sufficient length to ensure that an expandable device disclosed herein substantially completely seals a vessel or other tubular structure in a manner that reduces and/or prevents leakage of biological fluid from the vessel into the interstitial space. In aspects of this embodiment, an expandable device comprises an overlap region having a length of, e.g., about 0.1 mm, about 0.2 mm, about 0.3 mm, about 0.4 mm, about 0.5 mm, about 0.6 mm, about 0.7 mm, about 0.8 mm, about 0.9 mm, or about 1.0 mm. In other aspects of this embodiment, an expandable device comprises an overlap region having a length of, e.g., at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, or at least 1.0 mm. In yet other aspects of this embodiment, an expandable device comprises an overlap region having a length of, e.g., at most 0.1 mm, at most 0.2 mm, at most 0.3 mm, at most 0.4 mm, at most 0.5 mm, at most 0.6 mm, at most 0.7 mm, at most 0.8 mm, at most 0.9 mm, or at most 1.0 mm.

In still other aspects of this embodiment, an expandable device comprises an overlap region having a length of, e.g., about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.7 mm, about 0.1 mm to about 0.8 mm, about 0.1 mm to about 0.9 mm, about 0.1 mm to about 1.0 mm, about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1.0 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1.0 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1.0 mm, about 0.5 mm to about 0.6 mm, about 0.5 mm to about 0.7 mm, about 0.5 mm to about 0.8 mm, about 0.5 mm to about 0.9 mm, about 0.5 mm to about 1.0 mm, about 0.6 mm to about 0.7 mm, about 0.6 mm to about 0.8 mm, about 0.6 mm to about 0.9 mm, about 0.6 mm to about 1.0 mm, about 0.7 mm to about 0.8 mm, about 0.7 mm to about 0.9 mm, about 0.7 mm to about 1.0 mm, about 0.8 mm to about 0.9 mm, about 0.8 mm to about 1.0 mm, or about 0.9 mm to about 1.0 mm.

In aspects of this embodiment, an expandable device comprises an overlap region having a length of, e.g., about 0.1 cm, about 0.2 cm, about 0.3 cm, about 0.4 cm, about 0.5 cm, about 0.6 cm, about 0.7 cm, about 0.8 cm, about 0.9 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, about 2.5 cm, about 2.6 cm, about 2.7 cm, about 2.8 cm, about 2.9 cm, about 3.0 cm, about 3.1 cm, about 3.2 cm, about 3.3 cm, about 3.4 cm, about 3.5 cm, about 3.6 cm, about 3.7 cm, about 3.8 cm, about 3.9 cm, or about 4.0 cm. In other aspects of this embodiment, an expandable device comprises an overlap region having a length of, e.g., at least 0.1 cm, at least 0.2 cm, at least 0.3 cm, at least 0.4 cm, at least 0.5 cm, at least 0.6 cm, at least 0.7 cm, at least 0.8 cm, at least 0.9 cm, at least 1.0 cm, at least 1.1 cm, at least 1.2 cm, at least 1.3 cm, at least 1.4 cm, at least 1.5 cm, at least 1.6 cm, at least 1.7 cm, at least 1.8 cm, at least 1.9 cm, at least 2.0 cm, at least 2.1 cm, at least 2.2 cm, at least 2.3 cm, at least 2.4 cm, at least 2.5 cm, at least 2.6 cm, at least 2.7 cm, at least 2.8 cm, at least 2.9 cm, at least 3.0 cm, at least 3.1 cm, at least 3.2 cm, at least 3.3 cm, at least 3.4 cm, at least 3.5 cm, at least 3.6 cm, at least 3.7 cm, at least 3.8 cm, at least 3.9 cm, or at least 4.0 cm. In yet other aspects of this embodiment, an expandable device comprises an overlap region having a length of, e.g., at most 0.1 cm, at most 0.2 cm, at most 0.3 cm, at most 0.4 cm, at most 0.5 cm, at most 0.6 cm, at most 0.7 cm, at most 0.8 cm, at most 0.9 cm, at most 1.0 cm, at most 1.1 cm, at most 1.2 cm, at most 1.3 cm, at most 1.4 cm, at most 1.5 cm, at most 1.6 cm, at most 1.7 cm, at most 1.8 cm, at most 1.9 cm, at most 2.0 cm, at most 2.1 cm, at most 2.2 cm, at most 2.3 cm, at most 2.4 cm, at most 2.5 cm, at most 2.6 cm, at most 2.7 cm, at most 2.8 cm, at most 2.9 cm, at most 3.0 cm, at most 3.1 cm, at most 3.2 cm, at most 3.3 cm, at most 3.4 cm, at most 3.5 cm, at most 3.6 cm, at most 3.7 cm, at most 3.8 cm, at most 3.9 cm, or at most 4.0 cm.

In still other aspects of this embodiment, an expandable device comprises an overlap region having a length of, e.g., about 0.1 cm to about 0.5 cm, about 0.1 cm to about 0.75 cm, about 0.1 cm to about 1.0 cm, about 0.1 cm to about 1.25 cm, about 0.1 cm to about 1.5 cm, about 0.1 cm to about 1.75 cm, about 0.1 cm to about 2.0 cm, about 0.1 cm to about 2.25 cm, about 0.1 cm to about 2.5 cm, about 0.1 cm to about 2.75 cm, about 0.1 cm to about 3.0 cm, about 0.1 cm to about 3.25 cm, about 0.1 cm to about 3.5 cm, about 0.1 cm to about 3.75 cm, about 0.1 cm to about 4.0 cm, about 0.25 cm to about 0.5 cm, about 0.25 cm to about 0.75 cm, about 0.25 cm to about 1.0 cm, about 0.25 cm to about 1.25 cm, about 0.25 cm to about 1.5 cm, about 0.25 cm to about 1.75 cm, about 0.25 cm to about 2.0 cm, about 0.25 cm to about 2.25 cm, about 0.25 cm to about 2.5 cm, about 0.25 cm to about 2.75 cm, about 0.25 cm to about 3.0 cm, about 0.25 cm to about 3.25 cm, about 0.25 cm to about 3.5 cm, about 0.25 cm to about 3.75 cm, about 0.25 cm to about 4.0 cm, about 0.5 cm to about 0.75 cm, about 0.5 cm to about 1.0 cm, about 0.5 cm to about 1.25 cm, about 0.5 cm to about 1.5 cm, about 0.5 cm to about 1.75 cm, about 0.5 cm to about 2.0 cm, about 0.5 cm to about 2.25 cm, about 0.5 cm to about 2.5 cm, about 0.5 cm to about 2.75 cm, about 0.5 cm to about 3.0 cm, about 0.5 cm to about 3.25 cm, about 0.5 cm to about 3.5 cm, about 0.5 cm to about 3.75 cm, about 0.5 cm to about 4.0 cm, about 0.75 cm to about 1.0 cm, about 0.75 cm to about 1.25 cm, about 0.75 cm to about 1.5 cm, about 0.75 cm to about 1.75 cm, about 0.75 cm to about 2.0 cm, about 0.75 cm to about 2.25 cm, about 0.75 cm to about 2.5 cm, about 0.75 cm to about 2.75 cm, about 0.75 cm to about 3.0 cm, about 0.75 cm to about 3.25 cm, about 0.75 cm to about 3.5 cm, about 0.75 cm to about 3.75 cm, about 0.75 cm to about 4.0 cm, about 1.0 cm to about 1.25 cm, about 1.0 cm to about 1.5 cm, about 1.0 cm to about 1.75 cm, about 1.0 cm to about 2.0 cm, about 1.0 cm to about 2.25 cm, about 1.0 cm to about 2.5 cm, about 1.0 cm to about 2.75 cm, about 1.0 cm to about 3.0 cm, about 1.0 cm to about 3.25 cm, about 1.0 cm to about 3.5 cm, about 1.0 cm to about 3.75 cm, about 1.0 cm to about 4.0 cm, about 1.5 cm to about 2.0 cm, about 1.5 cm to about 2.25 cm, about 1.5 cm to about 2.5 cm, about 1.5 cm to about 2.75 cm, about 1.5 cm to about 3.0 cm, about 1.5 cm to about 3.25 cm, about 1.5 cm to about 3.5 cm, about 1.5 cm to about 3.75 cm, about 1.5 cm to about 4.0 cm, about 2.0 cm to about 2.25 cm, about 2.0 cm to about 2.5 cm, about 2.0 cm to about 2.75 cm, or about 2.0 cm to about 3.0 cm, about 2.0 cm to about 3.25 cm, about 2.0 cm to about 3.5 cm, about 2.0 cm to about 3.75 cm, about 2.0 cm to about 4.0 cm, about 2.5 cm to about 2.75 cm, or about 2.5 cm to about 3.0 cm, about 2.5 cm to about 3.25 cm, about 2.5 cm to about 3.5 cm, about 2.5 cm to about 3.75 cm, about 2.5 cm to about 4.0 cm, about 3.0 cm to about 3.25 cm, about 3.0 cm to about 3.5 cm, about 3.0 cm to about 3.75 cm, about 3.0 cm to about 4.0 cm, about 3.5 cm to about 3.75 cm, or about 3.5 cm to about 4.0 cm.

An expandable device disclosed herein may also comprise a port. A port disclosed herein allows for the delivery of a fluid into the lumen of the expandable device and thus the treated vessel or other tubular structure in order to, e.g., facilitate expansion of an expandable device disclosed herein or enable administration of a solution for medicinal purposes. A port disclosed herein may also serve as an anchoring point for an optional external sleeve, which fits over the tunica externa layer of the vessel. This port also allows for the continuous infusion of therapeutic agents, such as, e.g., anti-coagulants, the intermittent infusion of thrombolytics, or the continuous infusion of vasodialators, PDE inhibitors, or gasotransmitters, or administration of a contrasting or tracing agent (like a dye) useful for visualizing vessel structure and integrity such as, e.g., an angiogram, portal vein patency, or other blood vessel patency, neuronal patency, renal patency, urogenital patency, biliary patency, endocrine patency, respiratory patency, or other procedure used to detect and/or identify a vessel blockage. A port disclosed herein may be designed as a luer-slip or luer-lock to facilitate attachment of a syringe or similar device used to administer a fluid or therapeutic agent. In one embodiment, a port disclosed herein is configured to attach with a 1 cc, 2 cc, 3 cc, 4 cc, or 5 cc syringe or similar device used to administer a fluid or therapeutic agent. Thus, the expandable device disclosed herein is not only an anastomotic device but a device for the non-invasive ability to clear a clot or increase the flow in a vessel or other tubular structure without the need for a surgery in the operating room. Without limitation, medical personnel can utilize the port of an installed expandable device according to aspects of the present specification to instill heparin or other clot dissolving drug into a vessel lumen after surgery is completed. Typically, when anastomosis clots off after surgery, the surgeon has to bring the patient back for another surgery, open the wound, open up the anastomosis, instill heparin or other clot dissolving drug, then close up the surgical site again. With the port provided within the expandable device so as to be in fluid communication with the affected vessel lumen, this can be done at the bedside without a subsequent surgical procedure. Such use of the expandable device may be indicated for, e.g., micro-surgery. However, if adopted for larger blood vessels or other tubular structures, the overall device may be scaled up and/or modified in other respects as to its configuration, and the port particularly can be configured so as to accommodate a thrombectomy catheter as might be placed by a surgeon or interventional radiologist. It will be appreciated that other configurations and uses of a port as part of an expandable anastomosis device according to aspects of the present specification are possible, such that the exemplary designs are illustrative and non-limiting.

A port disclosed herein may also allow attachment of a suction device, typically through use of a hose or similar type connector. For example, a vacuum pump may be employed to apply a suction and a hose may connect the pump to an expandable device disclosed herein via a hose attached to a port disclosed herein.

A port disclosed herein may also may also allow attachment of a port cap. A port cap disclosed herein covers the channel of a port, thereby reducing or preventing leakage of a biological fluid from the vessel into the surrounding interstitial space. A port cap may be attached by any mechanism that will enable a secure attachment, such as, e.g., a press-fitting mechanism, a snap-on mechanism, or a screw mechanism.

In one embodiment, an expandable device disclosed herein comprises a single port. In one embodiment, an expandable device disclosed herein comprises a plurality of ports. In aspects of this embodiment, an expandable device disclosed herein comprises, e.g., one port, two ports, three ports, four ports, five ports, six ports, seven ports, eight ports, nine ports, or ten ports. In other aspects of this embodiment, an expandable device disclosed herein comprises, e.g., at least one port, at least two ports, at least three ports, at least four ports, at least five ports, at least six ports, at least seven ports, at least eight ports, at least nine ports, or at least ten ports. In yet other aspects of this embodiment, an expandable device disclosed herein comprises, e.g., at most one port, at most two ports, at most three ports, at most four ports, at most five ports, at most six ports, at most seven ports, at most eight ports, at most nine ports, or at most ten ports.

In still other aspects of this embodiment, an expandable device disclosed herein comprises, e.g., about 1 to about 2 ports, about 1 to about 3 ports, about 1 to about 4 ports, about 1 to about 5 ports, about 1 to about 6 ports, about 1 to about 7 ports, about 1 to about 8 ports, about 1 to about 9 ports, about 1 to about 10 ports, about 2 to about 3 ports, about 2 to about 4 ports, about 2 to about 5 ports, about 2 to about 6 ports, about 2 to about 7 ports, about 2 to about 8 ports, about 2 to about 9 ports, about 2 to about 10 ports, about 3 to about 4 ports, about 3 to about 5 ports, about 3 to about 6 ports, about 3 to about 7 ports, about 3 to about 8 ports, about 3 to about 9 ports, about 3 to about 10 ports, about 4 to about 5 ports, about 4 to about 6 ports, about 4 to about 7 ports, about 4 to about 8 ports, about 4 to about 9 ports, about 4 to about 10 ports, about 5 to about 6 ports, about 5 to about 7 ports, about 5 to about 8 ports, about 5 to about 9 ports, about 5 to about 10 ports, about 6 to about 7 ports, about 6 to about 8 ports, about 6 to about 9 ports, about 6 to about 10 ports, about 7 to about 8 ports, about 7 to about 9 ports, about 7 to about 10 ports, about 8 to about 9 ports, about 8 to about 10 ports, or about 9 to about 10 ports.

In one embodiment, an expandable device disclosed herein does not comprise a port.

An expandable device disclosed herein may also comprise one or more sensors. A sensor disclosed herein may be used to monitor physiological parameters, including, without limitation, fluid flow rate, fluid pressure, fluid viscosity, fluid contrast, luminal diameter of an expandable device, luminal diameter of vessel, structural integrity of an expandable device, structural integrity of vessel, detection of label or other tracing component in a fluid, and/or composition of fluid contained in vessel. In one embodiment, a sensor disclosed herein is a wireless ultrasound Doppler. Measured parameters may be sent using a wired connection or wirelessly and received directly or remotely to a computer or other device designed to receive and read the signals sent by a sensor disclosed herein. In one embodiment, the measured parameters can be assessed using a software program installed on a mobile electronic device such, as, e.g., a cell phone, a tablet, or a computer.

A sensor disclosed herein may be located on or integrated with any part of an expandable device disclosed herein. In aspects of this embodiment, a sensor disclosed herein may be located on or integrated with a tube assembly, a tube body, a port, a port cap, a channel of the port, or a lumen of a tube assembly. In other aspects of this embodiment, a sensor disclosed herein may be configured in a manner where it, or a portion thereof, is exposed to a fluid contained in a lumen of an expandable device disclosed herein. In yet other aspects of this embodiment, a sensor disclosed herein may be configured in a manner where it is not exposed to a fluid contained in a lumen of an expandable device disclosed herein.

In one embodiment, an expandable device disclosed herein comprises a single sensor. In one embodiment, an expandable device disclosed herein comprises a plurality of sensors. In aspects of this embodiment, an expandable device disclosed herein comprises, e.g., one sensor, two sensors, three sensors, four sensors, five sensors, six sensors, seven sensors, eight sensors, nine sensors, or ten sensors. In other aspects of this embodiment, an expandable device disclosed herein comprises, e.g., at least one sensor, at least two sensors, at least three sensors, at least four sensors, at least five sensors, at least six sensors, at least seven sensors, at least eight sensors, at least nine sensors, or at least ten sensors. In yet other aspects of this embodiment, an expandable device disclosed herein comprises, e.g., at most one sensor, at most two sensors, at most three sensors, at most four sensors, at most five sensors, at most six sensors, at most seven sensors, at most eight sensors, at most nine sensors, or at most ten sensors.

In still other aspects of this embodiment, an expandable device disclosed herein comprises, e.g., about 1 to about 2 sensors, about 1 to about 3 sensors, about 1 to about 4 sensors, about 1 to about 5 sensors, about 1 to about 6 sensors, about 1 to about 7 sensors, about 1 to about 8 sensors, about 1 to about 9 sensors, about 1 to about 10 sensors, about 2 to about 3 sensors, about 2 to about 4 sensors, about 2 to about 5 sensors, about 2 to about 6 sensors, about 2 to about 7 sensors, about 2 to about 8 sensors, about 2 to about 9 sensors, about 2 to about 10 sensors, about 3 to about 4 sensors, about 3 to about 5 sensors, about 3 to about 6 sensors, about 3 to about 7 sensors, about 3 to about 8 sensors, about 3 to about 9 sensors, about 3 to about 10 sensors, about 4 to about 5 sensors, about 4 to about 6 sensors, about 4 to about 7 sensors, about 4 to about 8 sensors, about 4 to about 9 sensors, about 4 to about 10 sensors, about 5 to about 6 sensors, about 5 to about 7 sensors, about 5 to about 8 sensors, about 5 to about 9 sensors, about 5 to about 10 sensors, about 6 to about 7 sensors, about 6 to about 8 sensors, about 6 to about 9 sensors, about 6 to about 10 sensors, about 7 to about 8 sensors, about 7 to about 9 sensors, about 7 to about 10 sensors, about 8 to about 9 sensors, about 8 to about 10 sensors, or about 9 to about 10 sensors.

An expandable device disclosed herein may also comprise one or more spikes. A spike disclosed herein comprises a pointed tip that is used to pierce the tunica externa and/or tunica externa media of a vessel in order to, e.g., facilitate attachment of a vessel to an expandable device disclosed herein, and/or anchor an expandable device to the surrounding interstitial space.

A spike disclosed herein may also allow attachment of a spike cap. A spike cap disclosed herein covers the tip of a spike, thereby protecting the surrounding interstitial tissue from damage. A spike cap may be attached by any mechanism that will enable a secure attachment, such as, e.g., a press-fitting mechanism, a snap-on mechanism, or a screw mechanism.

In one embodiment, an expandable device disclosed herein comprises a single spike. In one embodiment, an expandable device disclosed herein comprises a plurality of spikes. In aspects of this embodiment, an expandable device disclosed herein comprises, e.g., one spike, two spikes, three spikes, four spikes, five spikes, six spikes, seven spikes, eight spikes, nine spikes, or ten spikes. In other aspects of this embodiment, an expandable device disclosed herein comprises, e.g., at least one spike, at least two spikes, at least three spikes, at least four spikes, at least five spikes, at least six spikes, at least seven spikes, at least eight spikes, at least nine spikes, or at least ten spikes. In yet other aspects of this embodiment, an expandable device disclosed herein comprises, e.g., at most one spike, at most two spikes, at most three spikes, at most four spikes, at most five spikes, at most six spikes, at most seven spikes, at most eight spikes, at most nine spikes, or at most ten spikes.

In still other aspects of this embodiment, an expandable device disclosed herein comprises, e.g., about 1 to about 2 spikes, about 1 to about 3 spikes, about 1 to about 4 spikes, about 1 to about 5 spikes, about 1 to about 6 spikes, about 1 to about 7 spikes, about 1 to about 8 spikes, about 1 to about 9 spikes, about 1 to about 10 spikes, about 2 to about 3 spikes, about 2 to about 4 spikes, about 2 to about 5 spikes, about 2 to about 6 spikes, about 2 to about 7 spikes, about 2 to about 8 spikes, about 2 to about 9 spikes, about 2 to about 10 spikes, about 3 to about 4 spikes, about 3 to about 5 spikes, about 3 to about 6 spikes, about 3 to about 7 spikes, about 3 to about 8 spikes, about 3 to about 9 spikes, about 3 to about 10 spikes, about 4 to about 5 spikes, about 4 to about 6 spikes, about 4 to about 7 spikes, about 4 to about 8 spikes, about 4 to about 9 spikes, about 4 to about 10 spikes, about 5 to about 6 spikes, about 5 to about 7 spikes, about 5 to about 8 spikes, about 5 to about 9 spikes, about 5 to about 10 spikes, about 6 to about 7 spikes, about 6 to about 8 spikes, about 6 to about 9 spikes, about 6 to about 10 spikes, about 7 to about 8 spikes, about 7 to about 9 spikes, about 7 to about 10 spikes, about 8 to about 9 spikes, about 8 to about 10 spikes, or about 9 to about 10 spikes.

In one embodiment, an expandable device disclosed herein does not comprises any spikes. In one embodiment, an expandable device disclosed herein does not comprises any spikes, barbs or any other structure configured to pierce the tunica interna and/or cause trauma to a vessel.

An expandable device disclosed herein is designed to exist in at least two different shapes each comprising a volume: a relatively smaller or unexpanded first shape and a relatively larger or expanded second shape. Typically, where the expandable device is to be placed within the vessel or other tubular structure, the initial shape has a smaller volume than the volume of the expanded or second shape or that of the vessel lumen. Alternatively, where the expandable device is to be placed around the outside of a vessel or other tubular structure, the initial shape is typically larger in volume or is in an expanded state and thereby allows the vessel or lumen to be inserted within the expandable device. In either case, herein, the first shape and related first volume or first luminal diameter shall refer to the attributes of the device in its unexpanded state and the second shape and related second volume or second luminal diameter shall refer to the attributes of the device in its expanded state, at least in the exemplary embodiments. An increase in the diameter of the first and second luminal openings occurs with the change in volume of the device upon expansion, or from the first shape to the second shape or from the first volume to the second volume. As such, an expandable device disclosed herein has a first luminal diameter and a second luminal diameter. In addition, a first shape of an expandable device comprises a first volume that occurs at a first temperature and a second shape of an expandable device comprises a second volume that occurs at a second temperature. Thus, where an expandable device according to aspects of the specification is to be installed on the inside of a vessel or other tubular structure, the initial shape, or the configuration of the device at the time of initial insertion, correlates to the relatively smaller unexpanded first shape having a first volume and related first luminal diameter, wherein the device then expands to the relatively larger second shape having a second volume so as to seal and seat within the vessel or other tubular structure from the inside. And where an expandable device according to aspects of the specification is to be installed on the outside of a vessel or other tubular structure, the initial shape, or the configuration of the device at the time of initial insertion, correlates to the relatively larger expanded second shape having a second volume and related second luminal diameter, wherein the device then contracts to the relatively smaller first shape having a first volume to seal and seat on the vessel or other tubular structure from the outside.

In one embodiment a first shape comprises a first volume that is smaller than a second volume of the second shape. In aspects of this embodiment, a first shape of an expandable device comprises a first volume that is smaller than a second volume of a second shape of an expandable device by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In other aspect of this embodiment, a first shape of an expandable device comprises a first volume that is smaller than a second volume of a second shape of an expandable device by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In yet other aspect of this embodiment, a first shape of an expandable device comprises a first volume that is smaller than a second volume of a second shape of an expandable device by, e.g., at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.

In yet other aspect of this embodiment, a first shape of an expandable device comprises a first volume that is smaller than a second volume of a second shape of an expandable device by, e.g., about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 5% to about 80%, about 5% to about 85%, about 5% to about 90%, about 5% to about 95%, about 5% to about 100%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, about 10% to about 100%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 15% to about 80%, about 15% to about 85%, about 15% to about 90%, about 15% to about 95%, about 15% to about 100%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 20% to about 100%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 25% to about 85%, about 25% to about 90%, about 25% to about 95%, about 25% to about 100%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to about 100%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to about 100%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to about 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to about 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%.

In one embodiment, a second shape of an expandable device comprises a second volume that is larger than a first volume of a first shape of an expandable device. In aspects of this embodiment, a second shape of an expandable device comprises a second volume that is larger than a first volume of a first shape of an expandable device by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In other aspect of this embodiment, a second shape of an expandable device comprises a second volume that is larger than a first volume of a first shape of an expandable device by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In yet other aspect of this embodiment, a second shape of an expandable device comprises a second volume that is larger than a first volume of a first shape of an expandable device by, e.g., at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.

In yet other aspects of this embodiment, a second shape of an expandable device comprises a second volume that is larger than a first volume of a first shape of an expandable device by, e.g., about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 5% to about 80%, about 5% to about 85%, about 5% to about 90%, about 5% to about 95%, about 5% to about 100%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, about 10% to about 100%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 15% to about 80%, about 15% to about 85%, about 15% to about 90%, about 15% to about 95%, about 15% to about 100%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 20% to about 100%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 25% to about 85%, about 25% to about 90%, about 25% to about 95%, about 25% to about 100%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to about 100%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to about 100%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to about 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to about 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%.

In one embodiment, an expandable device disclosed herein has a first luminal diameter suitable to facilitate insertion of the expandable device into the lumen of a vessel. In aspects of this embodiment, an expandable device disclosed herein has a first luminal diameter of, e.g., about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, or about 4.0 mm. In other aspects of this embodiment, an expandable device disclosed herein has a first luminal diameter of, e.g., at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3.0 mm, at least 3.1 mm, at least 3.2 mm, at least 3.3 mm, at least 3.4 mm, at least 3.5 mm, at least 3.6 mm, at least 3.7 mm, at least 3.8 mm, at least 3.9 mm, or at least 4.0 mm. In yet other aspects of this embodiment, an expandable device disclosed herein has a first luminal diameter of, e.g., at most 0.1 mm, at most 0.2 mm, at most 0.3 mm, at most 0.4 mm, at most 0.5 mm, at most 0.6 mm, at most 0.7 mm, at most 0.8 mm, at most 0.9 mm, at most 1.0 mm, at most 1.1 mm, at most 1.2 mm, at most 1.3 mm, at most 1.4 mm, at most 1.5 mm, at most 1.6 mm, at most 1.7 mm, at most 1.8 mm, at most 1.9 mm, at most 2.0 mm, at most 2.1 mm, at most 2.2 mm, at most 2.3 mm, at most 2.4 mm, at most 2.5 mm, at most 2.6 mm, at most 2.7 mm, at most 2.8 mm, at most 2.9 mm, at most 3.0 mm, at most 3.1 mm, at most 3.2 mm, at most 3.3 mm, at most 3.4 mm, at most 3.5 mm, at most 3.6 mm, at most 3.7 mm, at most 3.8 mm, at most 3.9 mm, or at most 4.0 mm.

In still other aspects of this embodiment, an expandable device disclosed herein has a first luminal diameter of, e.g., about 0.1 mm to about 0.5 mm, about 0.1 mm to about 0.75 mm, about 0.1 mm to about 1.0 mm, about 0.1 mm to about 1.25 mm, about 0.1 mm to about 1.5 mm, about 0.1 mm to about 1.75 mm, about 0.1 mm to about 2.0 mm, about 0.1 mm to about 2.25 mm, about 0.1 mm to about 2.5 mm, about 0.1 mm to about 2.75 mm, about 0.1 mm to about 3.0 mm, about 0.1 mm to about 3.25 mm, about 0.1 mm to about 3.5 mm, about 0.1 mm to about 3.75 mm, about 0.1 mm to about 4.0 mm, about 0.25 mm to about 0.5 mm, about 0.25 mm to about 0.75 mm, about 0.25 mm to about 1.0 mm, about 0.25 mm to about 1.25 mm, about 0.25 mm to about 1.5 mm, about 0.25 mm to about 1.75 mm, about 0.25 mm to about 2.0 mm, about 0.25 mm to about 2.25 mm, about 0.25 mm to about 2.5 mm, about 0.25 mm to about 2.75 mm, about 0.25 mm to about 3.0 mm, about 0.25 mm to about 3.25 mm, about 0.25 mm to about 3.5 mm, about 0.25 mm to about 3.75 mm, about 0.25 mm to about 4.0 mm, about 0.5 mm to about 0.75 mm, about 0.5 mm to about 1.0 mm, about 0.5 mm to about 1.25 mm, about 0.5 mm to about 1.5 mm, about 0.5 mm to about 1.75 mm, about 0.5 mm to about 2.0 mm, about 0.5 mm to about 2.25 mm, about 0.5 mm to about 2.5 mm, about 0.5 mm to about 2.75 mm, about 0.5 mm to about 3.0 mm, about 0.5 mm to about 3.25 mm, about 0.5 mm to about 3.5 mm, about 0.5 mm to about 3.75 mm, about 0.5 mm to about 4.0 mm, about 0.75 mm to about 1.0 mm, about 0.75 mm to about 1.25 mm, about 0.75 mm to about 1.5 mm, about 0.75 mm to about 1.75 mm, about 0.75 mm to about 2.0 mm, about 0.75 mm to about 2.25 mm, about 0.75 mm to about 2.5 mm, about 0.75 mm to about 2.75 mm, about 0.75 mm to about 3.0 mm, about 0.75 mm to about 3.25 mm, about 0.75 mm to about 3.5 mm, about 0.75 mm to about 3.75 mm, about 0.75 mm to about 4.0 mm, about 1.0 mm to about 1.25 mm, about 1.0 mm to about 1.5 mm, about 1.0 mm to about 1.75 mm, about 1.0 mm to about 2.0 mm, about 1.0 mm to about 2.25 mm, about 1.0 mm to about 2.5 mm, about 1.0 mm to about 2.75 mm, about 1.0 mm to about 3.0 mm, about 1.0 mm to about 3.25 mm, about 1.0 mm to about 3.5 mm, about 1.0 mm to about 3.75 mm, about 1.0 mm to about 4.0 mm, about 1.5 mm to about 2.0 mm, about 1.5 mm to about 2.25 mm, about 1.5 mm to about 2.5 mm, about 1.5 mm to about 2.75 mm, about 1.5 mm to about 3.0 mm, about 1.5 mm to about 3.25 mm, about 1.5 mm to about 3.5 mm, about 1.5 mm to about 3.75 mm, about 1.5 mm to about 4.0 mm, about 2.0 mm to about 2.25 mm, about 2.0 mm to about 2.5 mm, about 2.0 mm to about 2.75 mm, or about 2.0 mm to about 3.0 mm, about 2.0 mm to about 3.25 mm, about 2.0 mm to about 3.5 mm, about 2.0 mm to about 3.75 mm, about 2.0 mm to about 4.0 mm, about 2.5 mm to about 2.75 mm, or about 2.5 mm to about 3.0 mm, about 2.5 mm to about 3.25 mm, about 2.5 mm to about 3.5 mm, about 2.5 mm to about 3.75 mm, about 2.5 mm to about 4.0 mm, about 3.0 mm to about 3.25 mm, about 3.0 mm to about 3.5 mm, about 3.0 mm to about 3.75 mm, about 3.0 mm to about 4.0 mm, about 3.5 mm to about 3.75 mm, or about 3.5 mm to about 4.0 mm.

In aspects of this embodiment, an expandable device disclosed herein has a first luminal diameter of, e.g., about 0.1 cm, about 0.2 cm, about 0.3 cm, about 0.4 cm, about 0.5 cm, about 0.6 cm, about 0.7 cm, about 0.8 cm, about 0.9 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, about 2.5 cm, about 2.6 cm, about 2.7 cm, about 2.8 cm, about 2.9 cm, about 3.0 cm, about 3.1 cm, about 3.2 cm, about 3.3 cm, about 3.4 cm, about 3.5 cm, about 3.6 cm, about 3.7 cm, about 3.8 cm, about 3.9 cm, or about 4.0 cm. In other aspects of this embodiment, an expandable device disclosed herein has a first luminal diameter of, e.g., at least 0.1 cm, at least 0.2 cm, at least 0.3 cm, at least 0.4 cm, at least 0.5 cm, at least 0.6 cm, at least 0.7 cm, at least 0.8 cm, at least 0.9 cm, at least 1.0 cm, at least 1.1 cm, at least 1.2 cm, at least 1.3 cm, at least 1.4 cm, at least 1.5 cm, at least 1.6 cm, at least 1.7 cm, at least 1.8 cm, at least 1.9 cm, at least 2.0 cm, at least 2.1 cm, at least 2.2 cm, at least 2.3 cm, at least 2.4 cm, at least 2.5 cm, at least 2.6 cm, at least 2.7 cm, at least 2.8 cm, at least 2.9 cm, at least 3.0 cm, at least 3.1 cm, at least 3.2 cm, at least 3.3 cm, at least 3.4 cm, at least 3.5 cm, at least 3.6 cm, at least 3.7 cm, at least 3.8 cm, at least 3.9 cm, or at least 4.0 cm. In yet other aspects of this embodiment, an expandable device disclosed herein has a first luminal diameter of, e.g., at most 0.1 cm, at most 0.2 cm, at most 0.3 cm, at most 0.4 cm, at most 0.5 cm, at most 0.6 cm, at most 0.7 cm, at most 0.8 cm, at most 0.9 cm, at most 1.0 cm, at most 1.1 cm, at most 1.2 cm, at most 1.3 cm, at most 1.4 cm, at most 1.5 cm, at most 1.6 cm, at most 1.7 cm, at most 1.8 cm, at most 1.9 cm, at most 2.0 cm, at most 2.1 cm, at most 2.2 cm, at most 2.3 cm, at most 2.4 cm, at most 2.5 cm, at most 2.6 cm, at most 2.7 cm, at most 2.8 cm, at most 2.9 cm, at most 3.0 cm, at most 3.1 cm, at most 3.2 cm, at most 3.3 cm, at most 3.4 cm, at most 3.5 cm, at most 3.6 cm, at most 3.7 cm, at most 3.8 cm, at most 3.9 cm, or at most 4.0 cm.

In still other aspects of this embodiment, an expandable device disclosed herein has a first luminal diameter of, e.g., about 0.1 cm to about 0.5 cm, about 0.1 cm to about 0.75 cm, about 0.1 cm to about 1.0 cm, about 0.1 cm to about 1.25 cm, about 0.1 cm to about 1.5 cm, about 0.1 cm to about 1.75 cm, about 0.1 cm to about 2.0 cm, about 0.1 cm to about 2.25 cm, about 0.1 cm to about 2.5 cm, about 0.1 cm to about 2.75 cm, about 0.1 cm to about 3.0 cm, about 0.1 cm to about 3.25 cm, about 0.1 cm to about 3.5 cm, about 0.1 cm to about 3.75 cm, about 0.1 cm to about 4.0 cm, about 0.25 cm to about 0.5 cm, about 0.25 cm to about 0.75 cm, about 0.25 cm to about 1.0 cm, about 0.25 cm to about 1.25 cm, about 0.25 cm to about 1.5 cm, about 0.25 cm to about 1.75 cm, about 0.25 cm to about 2.0 cm, about 0.25 cm to about 2.25 cm, about 0.25 cm to about 2.5 cm, about 0.25 cm to about 2.75 cm, about 0.25 cm to about 3.0 cm, about 0.25 cm to about 3.25 cm, about 0.25 cm to about 3.5 cm, about 0.25 cm to about 3.75 cm, about 0.25 cm to about 4.0 cm, about 0.5 cm to about 0.75 cm, about 0.5 cm to about 1.0 cm, about 0.5 cm to about 1.25 cm, about 0.5 cm to about 1.5 cm, about 0.5 cm to about 1.75 cm, about 0.5 cm to about 2.0 cm, about 0.5 cm to about 2.25 cm, about 0.5 cm to about 2.5 cm, about 0.5 cm to about 2.75 cm, about 0.5 cm to about 3.0 cm, about 0.5 cm to about 3.25 cm, about 0.5 cm to about 3.5 cm, about 0.5 cm to about 3.75 cm, about 0.5 cm to about 4.0 cm, about 0.75 cm to about 1.0 cm, about 0.75 cm to about 1.25 cm, about 0.75 cm to about 1.5 cm, about 0.75 cm to about 1.75 cm, about 0.75 cm to about 2.0 cm, about 0.75 cm to about 2.25 cm, about 0.75 cm to about 2.5 cm, about 0.75 cm to about 2.75 cm, about 0.75 cm to about 3.0 cm, about 0.75 cm to about 3.25 cm, about 0.75 cm to about 3.5 cm, about 0.75 cm to about 3.75 cm, about 0.75 cm to about 4.0 cm, about 1.0 cm to about 1.25 cm, about 1.0 cm to about 1.5 cm, about 1.0 cm to about 1.75 cm, about 1.0 cm to about 2.0 cm, about 1.0 cm to about 2.25 cm, about 1.0 cm to about 2.5 cm, about 1.0 cm to about 2.75 cm, about 1.0 cm to about 3.0 cm, about 1.0 cm to about 3.25 cm, about 1.0 cm to about 3.5 cm, about 1.0 cm to about 3.75 cm, about 1.0 cm to about 4.0 cm, about 1.5 cm to about 2.0 cm, about 1.5 cm to about 2.25 cm, about 1.5 cm to about 2.5 cm, about 1.5 cm to about 2.75 cm, about 1.5 cm to about 3.0 cm, about 1.5 cm to about 3.25 cm, about 1.5 cm to about 3.5 cm, about 1.5 cm to about 3.75 cm, about 1.5 cm to about 4.0 cm, about 2.0 cm to about 2.25 cm, about 2.0 cm to about 2.5 cm, about 2.0 cm to about 2.75 cm, or about 2.0 cm to about 3.0 cm, about 2.0 cm to about 3.25 cm, about 2.0 cm to about 3.5 cm, about 2.0 cm to about 3.75 cm, about 2.0 cm to about 4.0 cm, about 2.5 cm to about 2.75 cm, or about 2.5 cm to about 3.0 cm, about 2.5 cm to about 3.25 cm, about 2.5 cm to about 3.5 cm, about 2.5 cm to about 3.75 cm, about 2.5 cm to about 4.0 cm, about 3.0 cm to about 3.25 cm, about 3.0 cm to about 3.5 cm, about 3.0 cm to about 3.75 cm, about 3.0 cm to about 4.0 cm, about 3.5 cm to about 3.75 cm, or about 3.5 cm to about 4.0 cm.

In aspects of this embodiment, an expandable device disclosed herein has a first luminal diameter of, e.g., about 1 cm, about 2 cm, about 3 cm, about 4 cm, about 5 cm, about 6 cm, about 7 cm, about 8 cm, about 9 cm, or about 10 cm. In other aspects of this embodiment, an expandable device disclosed herein has a first luminal diameter of, e.g., at least 1 cm, at least 2 cm, at least 3 cm, at least 4 cm, at least 5 cm, at least 6 cm, at least 7 cm, at least 8 cm, at least 9 cm, or at least 10 cm. In yet other aspects of this embodiment, an expandable device disclosed herein has a first luminal diameter of, e.g., at most 1 cm, at most 2 cm, at most 3 cm, at most 4 cm, at most 5 cm, at most 6 cm, at most 7 cm, at most 8 cm, at most 9 cm, or at most 10 cm.

In still other aspects of this embodiment, an expandable device disclosed herein has a first luminal diameter of, e.g., about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 4 cm to about 5 cm, about 4 cm to about 6 cm, about 4 cm to about 7 cm, about 4 cm to about 8 cm, about 4 cm to about 9 cm, about 4 cm to about 10 cm, about 5 cm to about 6 cm, about 5 cm to about 7 cm, about 5 cm to about 8 cm, about 5 cm to about 9 cm, about 5 cm to about 10 cm, about 6 cm to about 7 cm, about 6 cm to about 8 cm, about 6 cm to about 9 cm, about 6 cm to about 10 cm, about 7 cm to about 8 cm, about 7 cm to about 9 cm, about 7 cm to about 10 cm, about 8 cm to about 9 cm, about 8 cm to about 10 cm, about 9 cm to about 10 cm.

In one embodiment, an expandable device disclosed herein has a second luminal diameter suitable to facilitate flow of a biological fluid through a vessel. In aspects of this embodiment, an expandable device disclosed herein has a second luminal diameter of, e.g., about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, or about 4.0 mm. In other aspects of this embodiment, an expandable device disclosed herein has a second luminal diameter of, e.g., at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3.0 mm, at least 3.1 mm, at least 3.2 mm, at least 3.3 mm, at least 3.4 mm, at least 3.5 mm, at least 3.6 mm, at least 3.7 mm, at least 3.8 mm, at least 3.9 mm, or at least 4.0 mm. In yet other aspects of this embodiment, an expandable device disclosed herein has a second luminal diameter of, e.g., at most 0.1 mm, at most 0.2 mm, at most 0.3 mm, at most 0.4 mm, at most 0.5 mm, at most 0.6 mm, at most 0.7 mm, at most 0.8 mm, at most 0.9 mm, at most 1.0 mm, at most 1.1 mm, at most 1.2 mm, at most 1.3 mm, at most 1.4 mm, at most 1.5 mm, at most 1.6 mm, at most 1.7 mm, at most 1.8 mm, at most 1.9 mm, at most 2.0 mm, at most 2.1 mm, at most 2.2 mm, at most 2.3 mm, at most 2.4 mm, at most 2.5 mm, at most 2.6 mm, at most 2.7 mm, at most 2.8 mm, at most 2.9 mm, at most 3.0 mm, at most 3.1 mm, at most 3.2 mm, at most 3.3 mm, at most 3.4 mm, at most 3.5 mm, at most 3.6 mm, at most 3.7 mm, at most 3.8 mm, at most 3.9 mm, or at most 4.0 mm.

In still other aspects of this embodiment, an expandable device disclosed herein has a second luminal diameter of, e.g., about 0.1 mm to about 0.5 mm, about 0.1 mm to about 0.75 mm, about 0.1 mm to about 1.0 mm, about 0.1 mm to about 1.25 mm, about 0.1 mm to about 1.5 mm, about 0.1 mm to about 1.75 mm, about 0.1 mm to about 2.0 mm, about 0.1 mm to about 2.25 mm, about 0.1 mm to about 2.5 mm, about 0.1 mm to about 2.75 mm, about 0.1 mm to about 3.0 mm, about 0.1 mm to about 3.25 mm, about 0.1 mm to about 3.5 mm, about 0.1 mm to about 3.75 mm, about 0.1 mm to about 4.0 mm, about 0.25 mm to about 0.5 mm, about 0.25 mm to about 0.75 mm, about 0.25 mm to about 1.0 mm, about 0.25 mm to about 1.25 mm, about 0.25 mm to about 1.5 mm, about 0.25 mm to about 1.75 mm, about 0.25 mm to about 2.0 mm, about 0.25 mm to about 2.25 mm, about 0.25 mm to about 2.5 mm, about 0.25 mm to about 2.75 mm, about 0.25 mm to about 3.0 mm, about 0.25 mm to about 3.25 mm, about 0.25 mm to about 3.5 mm, about 0.25 mm to about 3.75 mm, about 0.25 mm to about 4.0 mm, about 0.5 mm to about 0.75 mm, about 0.5 mm to about 1.0 mm, about 0.5 mm to about 1.25 mm, about 0.5 mm to about 1.5 mm, about 0.5 mm to about 1.75 mm, about 0.5 mm to about 2.0 mm, about 0.5 mm to about 2.25 mm, about 0.5 mm to about 2.5 mm, about 0.5 mm to about 2.75 mm, about 0.5 mm to about 3.0 mm, about 0.5 mm to about 3.25 mm, about 0.5 mm to about 3.5 mm, about 0.5 mm to about 3.75 mm, about 0.5 mm to about 4.0 mm, about 0.75 mm to about 1.0 mm, about 0.75 mm to about 1.25 mm, about 0.75 mm to about 1.5 mm, about 0.75 mm to about 1.75 mm, about 0.75 mm to about 2.0 mm, about 0.75 mm to about 2.25 mm, about 0.75 mm to about 2.5 mm, about 0.75 mm to about 2.75 mm, about 0.75 mm to about 3.0 mm, about 0.75 mm to about 3.25 mm, about 0.75 mm to about 3.5 mm, about 0.75 mm to about 3.75 mm, about 0.75 mm to about 4.0 mm, about 1.0 mm to about 1.25 mm, about 1.0 mm to about 1.5 mm, about 1.0 mm to about 1.75 mm, about 1.0 mm to about 2.0 mm, about 1.0 mm to about 2.25 mm, about 1.0 mm to about 2.5 mm, about 1.0 mm to about 2.75 mm, about 1.0 mm to about 3.0 mm, about 1.0 mm to about 3.25 mm, about 1.0 mm to about 3.5 mm, about 1.0 mm to about 3.75 mm, about 1.0 mm to about 4.0 mm, about 1.5 mm to about 2.0 mm, about 1.5 mm to about 2.25 mm, about 1.5 mm to about 2.5 mm, about 1.5 mm to about 2.75 mm, about 1.5 mm to about 3.0 mm, about 1.5 mm to about 3.25 mm, about 1.5 mm to about 3.5 mm, about 1.5 mm to about 3.75 mm, about 1.5 mm to about 4.0 mm, about 2.0 mm to about 2.25 mm, about 2.0 mm to about 2.5 mm, about 2.0 mm to about 2.75 mm, or about 2.0 mm to about 3.0 mm, about 2.0 mm to about 3.25 mm, about 2.0 mm to about 3.5 mm, about 2.0 mm to about 3.75 mm, about 2.0 mm to about 4.0 mm, about 2.5 mm to about 2.75 mm, or about 2.5 mm to about 3.0 mm, about 2.5 mm to about 3.25 mm, about 2.5 mm to about 3.5 mm, about 2.5 mm to about 3.75 mm, about 2.5 mm to about 4.0 mm, about 3.0 mm to about 3.25 mm, about 3.0 mm to about 3.5 mm, about 3.0 mm to about 3.75 mm, about 3.0 mm to about 4.0 mm, about 3.5 mm to about 3.75 mm, or about 3.5 mm to about 4.0 mm.

In aspects of this embodiment, an expandable device disclosed herein has a second luminal diameter of, e.g., about 0.1 cm, about 0.2 cm, about 0.3 cm, about 0.4 cm, about 0.5 cm, about 0.6 cm, about 0.7 cm, about 0.8 cm, about 0.9 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, about 2.5 cm, about 2.6 cm, about 2.7 cm, about 2.8 cm, about 2.9 cm, about 3.0 cm, about 3.1 cm, about 3.2 cm, about 3.3 cm, about 3.4 cm, about 3.5 cm, about 3.6 cm, about 3.7 cm, about 3.8 cm, about 3.9 cm, or about 4.0 cm. In other aspects of this embodiment, an expandable device disclosed herein has a second luminal diameter of, e.g., at least 0.1 cm, at least 0.2 cm, at least 0.3 cm, at least 0.4 cm, at least 0.5 cm, at least 0.6 cm, at least 0.7 cm, at least 0.8 cm, at least 0.9 cm, at least 1.0 cm, at least 1.1 cm, at least 1.2 cm, at least 1.3 cm, at least 1.4 cm, at least 1.5 cm, at least 1.6 cm, at least 1.7 cm, at least 1.8 cm, at least 1.9 cm, at least 2.0 cm, at least 2.1 cm, at least 2.2 cm, at least 2.3 cm, at least 2.4 cm, at least 2.5 cm, at least 2.6 cm, at least 2.7 cm, at least 2.8 cm, at least 2.9 cm, at least 3.0 cm, at least 3.1 cm, at least 3.2 cm, at least 3.3 cm, at least 3.4 cm, at least 3.5 cm, at least 3.6 cm, at least 3.7 cm, at least 3.8 cm, at least 3.9 cm, or at least 4.0 cm. In yet other aspects of this embodiment, an expandable device disclosed herein has a second luminal diameter of, e.g., at most 0.1 cm, at most 0.2 cm, at most 0.3 cm, at most 0.4 cm, at most 0.5 cm, at most 0.6 cm, at most 0.7 cm, at most 0.8 cm, at most 0.9 cm, at most 1.0 cm, at most 1.1 cm, at most 1.2 cm, at most 1.3 cm, at most 1.4 cm, at most 1.5 cm, at most 1.6 cm, at most 1.7 cm, at most 1.8 cm, at most 1.9 cm, at most 2.0 cm, at most 2.1 cm, at most 2.2 cm, at most 2.3 cm, at most 2.4 cm, at most 2.5 cm, at most 2.6 cm, at most 2.7 cm, at most 2.8 cm, at most 2.9 cm, at most 3.0 cm, at most 3.1 cm, at most 3.2 cm, at most 3.3 cm, at most 3.4 cm, at most 3.5 cm, at most 3.6 cm, at most 3.7 cm, at most 3.8 cm, at most 3.9 cm, or at most 4.0 cm.

In still other aspects of this embodiment, an expandable device disclosed herein has a second luminal diameter of, e.g., about 0.1 cm to about 0.5 cm, about 0.1 cm to about 0.75 cm, about 0.1 cm to about 1.0 cm, about 0.1 cm to about 1.25 cm, about 0.1 cm to about 1.5 cm, about 0.1 cm to about 1.75 cm, about 0.1 cm to about 2.0 cm, about 0.1 cm to about 2.25 cm, about 0.1 cm to about 2.5 cm, about 0.1 cm to about 2.75 cm, about 0.1 cm to about 3.0 cm, about 0.1 cm to about 3.25 cm, about 0.1 cm to about 3.5 cm, about 0.1 cm to about 3.75 cm, about 0.1 cm to about 4.0 cm, about 0.25 cm to about 0.5 cm, about 0.25 cm to about 0.75 cm, about 0.25 cm to about 1.0 cm, about 0.25 cm to about 1.25 cm, about 0.25 cm to about 1.5 cm, about 0.25 cm to about 1.75 cm, about 0.25 cm to about 2.0 cm, about 0.25 cm to about 2.25 cm, about 0.25 cm to about 2.5 cm, about 0.25 cm to about 2.75 cm, about 0.25 cm to about 3.0 cm, about 0.25 cm to about 3.25 cm, about 0.25 cm to about 3.5 cm, about 0.25 cm to about 3.75 cm, about 0.25 cm to about 4.0 cm, about 0.5 cm to about 0.75 cm, about 0.5 cm to about 1.0 cm, about 0.5 cm to about 1.25 cm, about 0.5 cm to about 1.5 cm, about 0.5 cm to about 1.75 cm, about 0.5 cm to about 2.0 cm, about 0.5 cm to about 2.25 cm, about 0.5 cm to about 2.5 cm, about 0.5 cm to about 2.75 cm, about 0.5 cm to about 3.0 cm, about 0.5 cm to about 3.25 cm, about 0.5 cm to about 3.5 cm, about 0.5 cm to about 3.75 cm, about 0.5 cm to about 4.0 cm, about 0.75 cm to about 1.0 cm, about 0.75 cm to about 1.25 cm, about 0.75 cm to about 1.5 cm, about 0.75 cm to about 1.75 cm, about 0.75 cm to about 2.0 cm, about 0.75 cm to about 2.25 cm, about 0.75 cm to about 2.5 cm, about 0.75 cm to about 2.75 cm, about 0.75 cm to about 3.0 cm, about 0.75 cm to about 3.25 cm, about 0.75 cm to about 3.5 cm, about 0.75 cm to about 3.75 cm, about 0.75 cm to about 4.0 cm, about 1.0 cm to about 1.25 cm, about 1.0 cm to about 1.5 cm, about 1.0 cm to about 1.75 cm, about 1.0 cm to about 2.0 cm, about 1.0 cm to about 2.25 cm, about 1.0 cm to about 2.5 cm, about 1.0 cm to about 2.75 cm, about 1.0 cm to about 3.0 cm, about 1.0 cm to about 3.25 cm, about 1.0 cm to about 3.5 cm, about 1.0 cm to about 3.75 cm, about 1.0 cm to about 4.0 cm, about 1.5 cm to about 2.0 cm, about 1.5 cm to about 2.25 cm, about 1.5 cm to about 2.5 cm, about 1.5 cm to about 2.75 cm, about 1.5 cm to about 3.0 cm, about 1.5 cm to about 3.25 cm, about 1.5 cm to about 3.5 cm, about 1.5 cm to about 3.75 cm, about 1.5 cm to about 4.0 cm, about 2.0 cm to about 2.25 cm, about 2.0 cm to about 2.5 cm, about 2.0 cm to about 2.75 cm, or about 2.0 cm to about 3.0 cm, about 2.0 cm to about 3.25 cm, about 2.0 cm to about 3.5 cm, about 2.0 cm to about 3.75 cm, about 2.0 cm to about 4.0 cm, about 2.5 cm to about 2.75 cm, or about 2.5 cm to about 3.0 cm, about 2.5 cm to about 3.25 cm, about 2.5 cm to about 3.5 cm, about 2.5 cm to about 3.75 cm, about 2.5 cm to about 4.0 cm, about 3.0 cm to about 3.25 cm, about 3.0 cm to about 3.5 cm, about 3.0 cm to about 3.75 cm, about 3.0 cm to about 4.0 cm, about 3.5 cm to about 3.75 cm, or about 3.5 cm to about 4.0 cm.

In aspects of this embodiment, an expandable device disclosed herein has a second luminal diameter of, e.g., about 1 cm, about 2 cm, about 3 cm, about 4 cm, about 5 cm, about 6 cm, about 7 cm, about 8 cm, about 9 cm, or about 10 cm. In other aspects of this embodiment, an expandable device disclosed herein has a second luminal diameter of, e.g., at least 1 cm, at least 2 cm, at least 3 cm, at least 4 cm, at least 5 cm, at least 6 cm, at least 7 cm, at least 8 cm, at least 9 cm, or at least 10 cm. In yet other aspects of this embodiment, an expandable device disclosed herein has a second luminal diameter of, e.g., at most 1 cm, at most 2 cm, at most 3 cm, at most 4 cm, at most 5 cm, at most 6 cm, at most 7 cm, at most 8 cm, at most 9 cm, or at most 10 cm.

In still other aspects of this embodiment, an expandable device disclosed herein has a second luminal diameter of, e.g., about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 4 cm to about 5 cm, about 4 cm to about 6 cm, about 4 cm to about 7 cm, about 4 cm to about 8 cm, about 4 cm to about 9 cm, about 4 cm to about 10 cm, about 5 cm to about 6 cm, about 5 cm to about 7 cm, about 5 cm to about 8 cm, about 5 cm to about 9 cm, about 5 cm to about 10 cm, about 6 cm to about 7 cm, about 6 cm to about 8 cm, about 6 cm to about 9 cm, about 6 cm to about 10 cm, about 7 cm to about 8 cm, about 7 cm to about 9 cm, about 7 cm to about 10 cm, about 8 cm to about 9 cm, about 8 cm to about 10 cm, about 9 cm to about 10 cm.

In one embodiment, a second shape of an expandable device comprises a second luminal diameter that is larger than a first luminal diameter of a first shape of an expandable device. In aspects of this embodiment, a second shape of an expandable device comprises a second luminal diameter that is larger than a first luminal diameter of a first shape of an expandable device by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In other aspect of this embodiment, a second shape of an expandable device comprises a second luminal diameter that is larger than a first luminal diameter of a first shape of an expandable device by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In yet other aspect of this embodiment, a second shape of an expandable device comprises a second luminal diameter that is larger than a first luminal diameter of a first shape of an expandable device by, e.g., at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.

In yet other aspects of this embodiment, a second shape of an expandable device comprises a second luminal diameter that is larger than a first luminal diameter of a first shape of an expandable device by, e.g., about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 5% to about 80%, about 5% to about 85%, about 5% to about 90%, about 5% to about 95%, about 5% to about 100%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, about 10% to about 100%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 15% to about 80%, about 15% to about 85%, about 15% to about 90%, about 15% to about 95%, about 15% to about 100%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 20% to about 100%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 25% to about 85%, about 25% to about 90%, about 25% to about 95%, about 25% to about 100%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to about 100%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to about 100%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to about 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to about 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%.

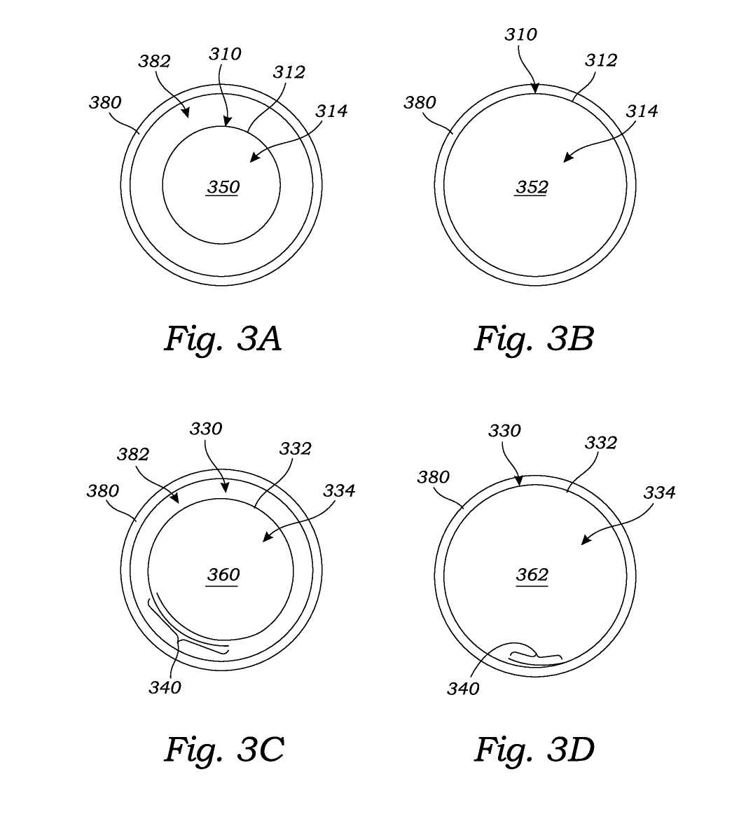

FIG. 3 illustrates one embodiment of a first shape comprising a first volume and a second shape comprising a second volume of an expandable device comprising a tube assembly disclosed herein first designed in a sealed configuration. FIG. 3A shows a cross-sectional or end schematic view of expandable device 310 having an intact tube body 312 and lumen 314 in a first shape comprising first volume 350 and inserted in lumen 382 of vessel 380. FIG. 3B shows a cross-sectional or end schematic view of expandable device 310 again having an intact tube body 312 and lumen 314 now in a second shape comprising second volume 352 enlarged in vessel 380. In the second shape comprising second volume 352, expandable device 310 has enlarged to fit substantially tightly against vessel 380, thereby resulting in lumen 382 being occupied by expandable device 310.

FIG. 3 also illustrates one embodiment of a first shape comprising a first volume and a second shape comprising a second volume of an expandable device comprising a tube assembly disclosed herein designed in an unsealed configuration. FIG. 3C shows a cross-sectional or end schematic view of expandable device 330 having a split tube body 332, overlap region 340, and lumen 334 in a first shape comprising first volume 360 inserted in lumen 382 of vessel 380. FIG. 3D shows a cross-sectional or end schematic view of expandable device 330 again having a split tube body 332, overlap region 340, and lumen 334 here in a second shape comprising second volume 362 enlarged in vessel 380. In the second shape comprising second volume 362, expandable device 330 has enlarged to fit tightly against vessel 380, thereby resulting in lumen 382 being occupied by expandable device 330. As shown in FIG. 3D, the overlap region 340 has decreased relative to that of expandable device 330 in its unexpanded state shown in FIG. 3C as expandable device 330 has expanded against the vessel 380.

In one embodiment, a first shape comprising a first volume of an expandable device disclosed herein occurs at a first temperature that is below a second temperature disclosed herein. In aspects of this embodiment, a first shape comprising a first volume of an expandable device disclosed herein occurs at a first temperature of, e.g., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., about 34° C., about 36° C., about 38° C., or about 40° C. In other aspects of this embodiment, a first shape comprising a first volume of an expandable device disclosed herein occurs at a first temperature of, e.g., at least 4° C., at least 6° C., at least 8° C., at least 10° C., at least 12° C., at least 14° C., at least 16° C., at least 18° C., at least 20° C., at least 22° C., at least 24° C., at least 26° C., at least 28° C., at least 30° C., at least 32° C., at least 34° C., at least 36° C., at least 38° C., or at least 40° C. In yet other aspects of this embodiment, a first shape comprising a first volume of an expandable device disclosed herein occurs at a first temperature of, e.g., 4° C. or below, 6° C. or below, 8° C. or below, 10° C. or below, 12° C. or below, 14° C. or below, 16° C. or below, 18° C. or below, 20° C. or below, 22° C. or below, 24° C. or below, 26° C. or below, 28° C. or below, 30° C. or below, 32° C. or below, 34° C. or below, 36° C. or below, 38° C. or below, or 40° C. or below.

In still other aspects of this embodiment, a first shape comprising a first volume of an expandable device disclosed herein occurs at a first temperature of, e.g., about 4° C. to about 6° C., about 4° C. to about 8° C., about 4° C. to about 10° C., about 4° C. to about 12° C., about 4° C. to about 14° C., about 4° C. to about 16° C., about 4° C. to about 18° C., about 4° C. to about 20° C., about 4° C. to about 22° C., about 4° C. to about 24° C., about 4° C. to about 26° C., about 4° C. to about 28° C., about 4° C. to about 30° C., about 4° C. to about 32° C., about 4° C. to about 34° C., about 4° C. to about 36° C., about 4° C. to about 38° C., about 4° C. to about 40° C., about 6° C. to about 8° C., about 6° C. to about 10° C., about 6° C. to about 12° C., about 6° C. to about 14° C., about 6° C. to about 16° C., about 6° C. to about 18° C., about 6° C. to about 20° C., about 6° C. to about 22° C., about 6° C. to about 24° C., about 6° C. to about 26° C., about 6° C. to about 28° C., about 6° C. to about 30° C., about 6° C. to about 32° C., about 6° C. to about 34° C., about 6° C. to about 36° C., about 6° C. to about 38° C., about 6° C. to about 40° C., about 8° C. to about 10° C., about 8° C. to about 12° C., about 8° C. to about 14° C., about 8° C. to about 16° C., about 8° C. to about 18° C., about 8° C. to about 20° C., about 8° C. to about 22° C., about 8° C. to about 24° C., about 8° C. to about 26° C., about 8° C. to about 28° C., about 8° C. to about 30° C., about 8° C. to about 32° C., about 8° C. to about 34° C., about 8° C. to about 36° C., about 8° C. to about 38° C., about 8° C. to about 40° C., about 10° C. to about 12° C., about 10° C. to about 14° C., about 10° C. to about 16° C., about 10° C. to about 18° C., about 10° C. to about 20° C., about 10° C. to about 22° C., about 10° C. to about 24° C., about 10° C. to about 26° C., about 10° C. to about 28° C., about 10° C. to about 30° C., about 10° C. to about 32° C., about 10° C. to about 34° C., about 10° C. to about 36° C., about 10° C. to about 38° C., about 10° C. to about 40° C., about 12° C. to about 14° C., about 12° C. to about 16° C., about 12° C. to about 18° C., about 12° C. to about 20° C., about 12° C. to about 22° C., about 12° C. to about 24° C., about 12° C. to about 26° C., about 12° C. to about 28° C., about 12° C. to about 30° C., about 12° C. to about 32° C., about 12° C. to about 34° C., about 12° C. to about 36° C., about 12° C. to about 38° C., about 12° C. to about 40° C., about 14° C. to about 16° C., about 14° C. to about 18° C., about 14° C. to about 20° C., about 14° C. to about 22° C., about 14° C. to about 24° C., about 14° C. to about 26° C., about 14° C. to about 28° C., about 14° C. to about 30° C., about 14° C. to about 32° C., about 14° C. to about 34° C., about 14° C. to about 36° C., about 14° C. to about 38° C., about 14° C. to about 40° C., about 16° C. to about 18° C., about 16° C. to about 20° C., about 16° C. to about 22° C., about 16° C. to about 24° C., about 16° C. to about 26° C., about 16° C. to about 28° C., about 16° C. to about 30° C., about 16° C. to about 32° C., about 16° C. to about 34° C., about 16° C. to about 36° C., about 16° C. to about 38° C., about 16° C. to about 40° C., about 18° C. to about 20° C., about 18° C. to about 22° C., about 18° C. to about 24° C., about 18° C. to about 26° C., about 18° C. to about 28° C., about 18° C. to about 30° C., about 18° C. to about 32° C., about 18° C. to about 34° C., about 18° C. to about 36° C., about 18° C. to about 38° C., about 18° C. to about 40° C., about 20° C. to about 22° C., about 20° C. to about 24° C., about 20° C. to about 26° C., about 20° C. to about 28° C., about 20° C. to about 30° C., about 20° C. to about 32° C., about 20° C. to about 34° C., about 20° C. to about 36° C., about 20° C. to about 38° C., about 20° C. to about 40° C., about 22° C. to about 24° C., about 22° C. to about 26° C., about 22° C. to about 28° C., about 22° C. to about 30° C., about 22° C. to about 32° C., about 22° C. to about 34° C., about 22° C. to about 36° C., about 22° C. to about 38° C., about 22° C. to about 40° C., about 24° C. to about 26° C., about 24° C. to about 28° C., about 24° C. to about 30° C., about 24° C. to about 32° C., about 24° C. to about 34° C., about 24° C. to about 36° C., about 24° C. to about 38° C., about 24° C. to about 40° C., about 26° C. to about 28° C., about 26° C. to about 30° C., about 26° C. to about 32° C., about 26° C. to about 34° C., about 26° C. to about 36° C., about 26° C. to about 38° C., about 26° C. to about 40° C., about 28° C. to about 30° C., about 28° C. to about 32° C., about 28° C. to about 34° C., about 28° C. to about 36° C., about 28° C. to about 38° C., about 28° C. to about 40° C., about 30° C. to about 32° C., about 30° C. to about 34° C., about 30° C. to about 36° C., about 30° C. to about 38° C., about 30° C. to about 40° C., about 32° C. to about 34° C., about 32° C. to about 36° C., about 32° C. to about 38° C., about 32° C. to about 40° C., about 34° C. to about 36° C., about 34° C. to about 38° C., about 34° C. to about 40° C., about 36° C. to about 38° C., about 36° C. to about 40° C., or about 38° C. to about 40° C.

In one embodiment, a second shape comprising a second volume of an expandable device disclosed herein occurs at a second temperature that is above a first temperature disclosed herein. In aspects of this embodiment, a second shape comprising a second volume of an expandable device disclosed herein occurs at a second temperature of, e.g., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In other aspects of this embodiment, a second shape comprising a second volume of an expandable device disclosed herein occurs at a second temperature, e.g., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., or at least 50° C. In yet other aspects of this embodiment, a second shape comprising a second volume of an expandable device disclosed herein occurs at a second temperature, e.g., 24° C. or above, 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, 45° C. or above, 46° C. or above, 47° C. or above, 48° C. or above, 49° C. or above, or 50° C. or above.

In still other aspects of this embodiment, a second shape comprising a second volume of an expandable device disclosed herein occurs at a second temperature, e.g., about 24° C. to about 26° C., about 24° C. to about 28° C., about 24° C. to about 30° C., about 24° C. to about 32° C., about 24° C. to about 34° C., about 24° C. to about 36° C., about 24° C. to about 38° C., about 24° C. to about 40° C., about 24° C. to about 42° C., about 24° C. to about 44° C., about 24° C. to about 46° C., about 24° C. to about 48° C., about 24° C. to about 50° C., about 25° C. to about 26° C., about 25° C. to about 28° C., about 25° C. to about 30° C., about 25° C. to about 32° C., about 25° C. to about 34° C., about 25° C. to about 36° C., about 25° C. to about 38° C., about 25° C. to about 40° C., about 25° C. to about 42° C., about 25° C. to about 44° C., about 25° C. to about 46° C., about 25° C. to about 48° C., about 25° C. to about 50° C., about 26° C. to about 28° C., about 26° C. to about 30° C., about 26° C. to about 32° C., about 26° C. to about 34° C., about 26° C. to about 36° C., about 26° C. to about 38° C., about 26° C. to about 40° C., about 26° C. to about 42° C., about 26° C. to about 44° C., about 26° C. to about 46° C., about 26° C. to about 48° C., about 26° C. to about 50° C., about 27° C. to about 28° C., about 27° C. to about 30° C., about 27° C. to about 32° C., about 27° C. to about 34° C., about 27° C. to about 36° C., about 27° C. to about 38° C., about 27° C. to about 40° C., about 27° C. to about 42° C., about 27° C. to about 44° C., about 27° C. to about 46° C., about 27° C. to about 48° C., about 27° C. to about 50° C., about 28° C. to about 30° C., about 28° C. to about 32° C., about 28° C. to about 34° C., about 28° C. to about 36° C., about 28° C. to about 38° C., about 28° C. to about 40° C., about 28° C. to about 42° C., about 28° C. to about 44° C., about 28° C. to about 46° C., about 28° C. to about 48° C., about 28° C. to about 50° C., about 29° C. to about 30° C., about 29° C. to about 32° C., about 29° C. to about 34° C., about 29° C. to about 36° C., about 29° C. to about 38° C., about 29° C. to about 40° C., about 29° C. to about 42° C., about 29° C. to about 44° C., about 29° C. to about 46° C., about 29° C. to about 48° C., about 29° C. to about 50° C., about 30° C. to about 32° C., about 30° C. to about 34° C., about 30° C. to about 36° C., about 30° C. to about 38° C., about 30° C. to about 40° C., about 30° C. to about 42° C., about 30° C. to about 44° C., about 30° C. to about 46° C., about 30° C. to about 48° C., about 30° C. to about 50° C., about 31° C. to about 32° C., about 31° C. to about 34° C., about 31° C. to about 36° C., about 31° C. to about 38° C., about 31° C. to about 40° C., about 31° C. to about 42° C., about 31° C. to about 44° C., about 31° C. to about 46° C., about 31° C. to about 48° C., about 31° C. to about 50° C., about 32° C. to about 34° C., about 32° C. to about 36° C., about 32° C. to about 38° C., about 32° C. to about 40° C., about 32° C. to about 42° C., about 32° C. to about 44° C., about 32° C. to about 46° C., about 32° C. to about 48° C., about 32° C. to about 50° C., about 33° C. to about 34° C., about 33° C. to about 36° C., about 33° C. to about 38° C., about 33° C. to about 40° C., about 33° C. to about 42° C., about 33° C. to about 44° C., about 33° C. to about 46° C., about 33° C. to about 48° C., about 33° C. to about 50° C., about 34° C. to about 36° C., about 34° C. to about 38° C., about 34° C. to about 40° C., about 34° C. to about 42° C., about 34° C. to about 44° C., about 34° C. to about 46° C., about 34° C. to about 48° C., about 34° C. to about 50° C., about 35° C. to about 36° C., about 35° C. to about 38° C., about 35° C. to about 40° C., about 35° C. to about 42° C., about 35° C. to about 44° C., about 35° C. to about 46° C., about 35° C. to about 48° C., about 35° C. to about 50° C., about 36° C. to about 38° C., about 36° C. to about 40° C., about 37° C. to about 38° C., about 37° C. to about 40° C., about 37° C. to about 42° C., about 37° C. to about 44° C., about 37° C. to about 46° C., about 37° C. to about 48° C., about 37° C. to about 50° C., about 38° C. to about 40° C., about 38° C. to about 42° C., about 38° C. to about 44° C., about 38° C. to about 46° C., about 38° C. to about 48° C., about 38° C. to about 50° C., about 39° C. to about 40° C., about 39° C. to about 42° C., about 39° C. to about 44° C., about 39° C. to about 46° C., about 39° C. to about 48° C., about 39° C. to about 50° C., about 40° C. to about 42° C., about 40° C. to about 44° C., about 40° C. to about 46° C., about 40° C. to about 48° C., about 40° C. to about 50° C., about 41° C. to about 42° C., about 41° C. to about 44° C., about 41° C. to about 46° C., about 41° C. to about 48° C., about 41° C. to about 50° C., about 42° C. to about 44° C., about 42° C. to about 46° C., about 42° C. to about 48° C., about 42° C. to about 50° C., about 43° C. to about 44° C., about 43° C. to about 46° C., about 43° C. to about 48° C., about 43° C. to about 50° C., about 44° C. to about 46° C., about 44° C. to about 48° C., about 44° C. to about 50° C., about 45° C. to about 46° C., about 45° C. to about 48° C., about 45° C. to about 50° C., about 46° C. to about 48° C., about 46° C. to about 50° C., about 47° C. to about 48° C., about 47° C. to about 50° C., or about 48° C. to about 50° C.

In one embodiment, a first temperature disclosed herein is lower than a second temperature disclosed herein. In aspects of this embodiment, a first temperature is lower than a second temperature by, e.g., about 2° C., about 4° C., about 6° C., about 8° C., about 10° C., about 12° C., about 14° C., about 16° C., about 18° C., about 20° C., about 22° C., about 24° C., about 26° C., about 28° C., or about 30° C. In other aspects of this embodiment, a first temperature is lower than a second temperature by, e.g., at least 2° C., at least 4° C., at least 6° C., at least 8° C., at least 10° C., at least 12° C., at least 14° C., at least 16° C., at least 18° C., at least 20° C., at least 22° C., at least 24° C., at least 26° C., at least 28° C., or at least 30° C. In yet other aspects of this embodiment, a first temperature is lower than a second temperature by, e.g., at most 2° C., at most 4° C., at most 6° C., at most 8° C., at most 10° C., at most 12° C., at most 14° C., at most 16° C., at most 18° C., at most 20° C., at most 22° C., at most 24° C., at most 26° C., at most 28° C., or at most 30° C.

In still other aspects of this embodiment, a first temperature is lower than a second temperature by, e.g., about 2° C. to about 4° C., about 2° C. to about 6° C., about 2° C. to about 8° C., about 2° C. to about 10° C., about 2° C. to about 12° C., about 2° C. to about 14° C., about 2° C. to about 16° C., about 2° C. to about 18° C., about 2° C. to about 20° C., about 2° C. to about 22° C., about 2° C. to about 24° C., about 2° C. to about 26° C., about 2° C. to about 28° C., about 2° C. to about 30° C., about 4° C. to about 6° C., about 4° C. to about 8° C., about 4° C. to about 10° C., about 4° C. to about 12° C., about 4° C. to about 14° C., about 4° C. to about 16° C., about 4° C. to about 18° C., about 4° C. to about 20° C., about 4° C. to about 22° C., about 4° C. to about 24° C., about 4° C. to about 26° C., about 4° C. to about 28° C., about 4° C. to about 30° C., about 6° C. to about 8° C., about 6° C. to about 10° C., about 6° C. to about 12° C., about 6° C. to about 14° C., about 6° C. to about 16° C., about 6° C. to about 18° C., about 6° C. to about 20° C., about 6° C. to about 22° C., about 6° C. to about 24° C., about 6° C. to about 26° C., about 6° C. to about 28° C., about 6° C. to about 30° C., about 8° C. to about 10° C., about 8° C. to about 12° C., about 8° C. to about 14° C., about 8° C. to about 16° C., about 8° C. to about 18° C., about 8° C. to about 20° C., about 8° C. to about 22° C., about 8° C. to about 24° C., about 8° C. to about 26° C., about 8° C. to about 28° C., about 8° C. to about 30° C., about 10° C. to about 12° C., about 10° C. to about 14° C., about 10° C. to about 16° C., about 10° C. to about 18° C., about 10° C. to about 20° C., about 10° C. to about 22° C., about 10° C. to about 24° C., about 10° C. to about 26° C., about 10° C. to about 28° C., about 10° C. to about 30° C., about 12° C. to about 14° C., about 12° C. to about 16° C., about 12° C. to about 18° C., about 12° C. to about 20° C., about 12° C. to about 22° C., about 12° C. to about 24° C., about 12° C. to about 26° C., about 12° C. to about 28° C., about 12° C. to about 30° C., about 14° C. to about 16° C., about 14° C. to about 18° C., about 14° C. to about 20° C., about 14° C. to about 22° C., about 14° C. to about 24° C., about 14° C. to about 26° C., about 14° C. to about 28° C., about 14° C. to about 30° C., about 16° C. to about 18° C., about 16° C. to about 20° C., about 16° C. to about 22° C., about 16° C. to about 24° C., about 16° C. to about 26° C., about 16° C. to about 28° C., about 16° C. to about 30° C., about 18° C. to about 20° C., about 18° C. to about 22° C., about 18° C. to about 24° C., about 18° C. to about 26° C., about 18° C. to about 28° C., about 18° C. to about 30° C., about 20° C. to about 22° C., about 20° C. to about 24° C., about 20° C. to about 26° C., about 20° C. to about 28° C., about 20° C. to about 30° C., about 22° C. to about 24° C., about 22° C. to about 26° C., about 22° C. to about 28° C., about 22° C. to about 30° C., about 24° C. to about 26° C., about 24° C. to about 28° C., about 24° C. to about 30° C., about 26° C. to about 28° C., about 26° C. to about 30° C., or about 28° C. to about 30° C.

In one embodiment, an expandable device disclosed herein having a first shape comprising a first volume at room temperature and a second shape comprising a second volume at body response temperature, wherein the first volume is smaller than the second volume.

In aspects of this embodiment, an expandable device disclosed herein having a first shape comprising a first volume at 25° C. or below and a second shape comprising a second volume at 29° C. or above, wherein the first volume is smaller than the second volume. In aspects of this embodiment, an expandable device disclosed herein having a first shape comprising a first volume at 25° C. or below and a second shape comprising a second volume at 30° C. or above, wherein the first volume is smaller than the second volume. In aspects of this embodiment, an expandable device disclosed herein having a first shape comprising a first volume at 25° C. or below and a second shape comprising a second volume at 32° C. or above, wherein the first volume is smaller than the second volume. In aspects of this embodiment, an expandable device disclosed herein having a first shape comprising a first volume at 25° C. or below and a second shape comprising a second volume at 34° C. or above, wherein the first volume is smaller than the second volume. In aspects of this embodiment, an expandable device disclosed herein having a first shape comprising a first volume at 25° C. or below and a second shape comprising a second volume at 35° C. or above, wherein the first volume is smaller than the second volume. In aspects of this embodiment, an expandable device disclosed herein having a first shape comprising a first volume at 25° C. or below and a second shape comprising a second volume at 36° C. or above, wherein the first volume is smaller than the second volume.

In aspects of this embodiment, an expandable device disclosed herein having a first shape comprising a first volume at 26° C. or below and a second shape comprising a second volume at 30° C. or above, wherein the first volume is smaller than the second volume. In aspects of this embodiment, an expandable device disclosed herein having a first shape comprising a first volume at 26° C. or below and a second shape comprising a second volume at 32° C. or above, wherein the first volume is smaller than the second volume. In aspects of this embodiment, an expandable device disclosed herein having a first shape comprising a first volume at 26° C. or below and a second shape comprising a second volume at 34° C. or above, wherein the first volume is smaller than the second volume. In aspects of this embodiment, an expandable device disclosed herein having a first shape comprising a first volume at 26° C. or below and a second shape comprising a second volume at 35° C. or above, wherein the first volume is smaller than the second volume. In aspects of this embodiment, an expandable device disclosed herein having a first shape comprising a first volume at 26° C. or below and a second shape comprising a second volume at 36° C. or above, wherein the first volume is smaller than the second volume.

In aspects of this embodiment, an expandable device disclosed herein having a first shape comprising a first volume at 27° C. or below and a second shape comprising a second volume at 31° C. or above, wherein the first volume is smaller than the second volume. In aspects of this embodiment, an expandable device disclosed herein having a first shape comprising a first volume at 27° C. or below and a second shape comprising a second volume at 32° C. or above, wherein the first volume is smaller than the second volume. In aspects of this embodiment, an expandable device disclosed herein having a first shape comprising a first volume at 27° C. or below and a second shape comprising a second volume at 34° C. or above, wherein the first volume is smaller than the second volume. In aspects of this embodiment, an expandable device disclosed herein having a first shape comprising a first volume at 27° C. or below and a second shape comprising a second volume at 35° C. or above, wherein the first volume is smaller than the second volume. In aspects of this embodiment, an expandable device disclosed herein having a first shape comprising a first volume at 27° C. or below and a second shape comprising a second volume at 36° C. or above, wherein the first volume is smaller than the second volume.

In aspects of this embodiment, an expandable device disclosed herein having a first shape comprising a first volume at 28° C. or below and a second shape comprising a second volume at 32° C. or above, wherein the first volume is smaller than the second volume. In aspects of this embodiment, an expandable device disclosed herein having a first shape comprising a first volume at 28° C. or below and a second shape comprising a second volume at 34° C. or above, wherein the first volume is smaller than the second volume. In aspects of this embodiment, an expandable device disclosed herein having a first shape comprising a first volume at 28° C. or below and a second shape comprising a second volume at 35° C. or above, wherein the first volume is smaller than the second volume. In aspects of this embodiment, an expandable device disclosed herein having a first shape comprising a first volume at 28° C. or below and a second shape comprising a second volume at 36° C. or above, wherein the first volume is smaller than the second volume.

In aspects of this embodiment, an expandable device disclosed herein having a first shape comprising a first volume at 29° C. or below and a second shape comprising a second volume at 33° C. or above, wherein the first volume is smaller than the second volume. In aspects of this embodiment, an expandable device disclosed herein having a first shape comprising a first volume at 29° C. or below and a second shape comprising a second volume at 34° C. or above, wherein the first volume is smaller than the second volume. In aspects of this embodiment, an expandable device disclosed herein having a first shape comprising a first volume at 29° C. or below and a second shape comprising a second volume at 35° C. or above, wherein the first volume is smaller than the second volume. In aspects of this embodiment, an expandable device disclosed herein having a first shape comprising a first volume at 29° C. or below and a second shape comprising a second volume at 36° C. or above, wherein the first volume is smaller than the second volume.

In aspects of this embodiment, an expandable device disclosed herein having a first shape comprising a first volume at 25° C. or below and a second shape comprising a second volume at 29° C. or above, wherein the first volume is smaller than the second volume by at least 5%. In aspects of this embodiment, an expandable device disclosed herein having a first shape comprising a first volume at 25° C. or below and a second shape comprising a second volume at 30° C. or above, wherein the first volume is smaller than the second volume by at least 5%. In aspects of this embodiment, an expandable device disclosed herein having a first shape comprising a first volume at 25° C. or below and a second shape comprising a second volume at 32° C. or above, wherein the first volume is smaller than the second volume by at least 5%. In aspects of this embodiment, an expandable device disclosed herein having a first shape comprising a first volume at 25° C. or below and a second shape comprising a second volume at 34° C. or above, wherein the first volume is smaller than the second volume by at least 5%. In aspects of this embodiment, an expandable device disclosed herein having a first shape comprising a first volume at 25° C. or below and a second shape comprising a second volume at 35° C. or above, wherein the first volume is smaller than the second volume by at least 5%. In aspects of this embodiment, an expandable device disclosed herein having a first shape comprising a first volume at 25° C. or below and a second shape comprising a second volume at 36° C. or above, wherein the first volume is smaller than the second volume by at least 5%.

In aspects of this embodiment, an expandable device disclosed herein having a first shape comprising a first volume at 26° C. or below and a second shape comprising a second volume at 30° C. or above, wherein the first volume is smaller than the second volume by at least 5%. In aspects of this embodiment, an expandable device disclosed herein having a first shape comprising a first volume at 26° C. or below and a second shape comprising a second volume at 32° C. or above, wherein the first volume is smaller than the second volume by at least 5%. In aspects of this embodiment, an expandable device disclosed herein having a first shape comprising a first volume at 26° C. or below and a second shape comprising a second volume at 34° C. or above, wherein the first volume is smaller than the second volume by at least 5%. In aspects of this embodiment, an expandable device disclosed herein having a first shape comprising a first volume at 26° C. or below and a second shape comprising a second volume at 35° C. or above, wherein the first volume is smaller than the second volume by at least 5%. In aspects of this embodiment, an expandable device disclosed herein having a first shape comprising a first volume at 26° C. or below and a second shape comprising a second volume at 36° C. or above, wherein the first volume is smaller than the second volume by at least 5%.

In aspects of this embodiment, an expandable device disclosed herein having a first shape comprising a first volume at 27° C. or below and a second shape comprising a second volume at 31° C. or above, wherein the first volume is smaller than the second volume by at least 5%. In aspects of this embodiment, an expandable device disclosed herein having a first shape comprising a first volume at 27° C. or below and a second shape comprising a second volume at 32° C. or above, wherein the first volume is smaller than the second volume by at least 5%. In aspects of this embodiment, an expandable device disclosed herein having a first shape comprising a first volume at 27° C. or below and a second shape comprising a second volume at 34° C. or above, wherein the first volume is smaller than the second volume by at least 5%. In aspects of this embodiment, an expandable device disclosed herein having a first shape comprising a first volume at 27° C. or below and a second shape comprising a second volume at 35° C. or above, wherein the first volume is smaller than the second volume by at least 5%. In aspects of this embodiment, an expandable device disclosed herein having a first shape comprising a first volume at 27° C. or below and a second shape comprising a second volume at 36° C. or above, wherein the first volume is smaller than the second volume by at least 5%.

In aspects of this embodiment, an expandable device disclosed herein having a first shape comprising a first volume at 28° C. or below and a second shape comprising a second volume at 32° C. or above, wherein the first volume is smaller than the second volume by at least 5%. In aspects of this embodiment, an expandable device disclosed herein having a first shape comprising a first volume at 28° C. or below and a second shape comprising a second volume at 34° C. or above, wherein the first volume is smaller than the second volume by at least 5%. In aspects of this embodiment, an expandable device disclosed herein having a first shape comprising a first volume at 28° C. or below and a second shape comprising a second volume at 35° C. or above, wherein the first volume is smaller than the second volume by at least 5%. In aspects of this embodiment, an expandable device disclosed herein having a first shape comprising a first volume at 28° C. or below and a second shape comprising a second volume at 36° C. or above, wherein the first volume is smaller than the second volume by at least 5%.

In aspects of this embodiment, an expandable device disclosed herein having a first shape comprising a first volume at 29° C. or below and a second shape comprising a second volume at 33° C. or above, wherein the first volume is smaller than the second volume by at least 5%. In aspects of this embodiment, an expandable device disclosed herein having a first shape comprising a first volume at 29° C. or below and a second shape comprising a second volume at 34° C. or above, wherein the first volume is smaller than the second volume by at least 5%. In aspects of this embodiment, an expandable device disclosed herein having a first shape comprising a first volume at 29° C. or below and a second shape comprising a second volume at 36° C. or above, wherein the first volume is smaller than the second volume by at least 5%. In aspects of this embodiment, an expandable device disclosed herein having a first shape comprising a first volume at 29° C. or below and a second shape comprising a second volume at 35° C. or above, wherein the first volume is smaller than the second volume by at least 5%.

In one embodiment, an expandable device disclosed herein having a first shape comprising a first volume at refrigerated temperature and a second shape comprising a second volume at body response temperature, wherein the first volume is smaller than the second volume. In aspects of this embodiment, an expandable device disclosed herein having a first shape comprising a first volume at 10° C. or below and a second shape comprising a second volume at 29° C. or above, wherein the first volume is smaller than the second volume. In aspects of this embodiment, an expandable device disclosed herein having a first shape comprising a first volume at 10° C. or below and a second shape comprising a second volume at 30° C. or above, wherein the first volume is smaller than the second volume. In aspects of this embodiment, an expandable device disclosed herein having a first shape comprising a first volume at 10° C. or below and a second shape comprising a second volume at 32° C. or above, wherein the first volume is smaller than the second volume. In aspects of this embodiment, an expandable device disclosed herein having a first shape comprising a first volume at 10° C. or below and a second shape comprising a second volume at 34° C. or above, wherein the first volume is smaller than the second volume. In aspects of this embodiment, an expandable device disclosed herein having a first shape comprising a first volume at 10° C. or below and a second shape comprising a second volume at 35° C. or above, wherein the first volume is smaller than the second volume. In aspects of this embodiment, an expandable device disclosed herein having a first shape comprising a first volume at 10° C. or below and a second shape comprising a second volume at 36° C. or above, wherein the first volume is smaller than the second volume.

In aspects of this embodiment, an expandable device disclosed herein having a first shape comprising a first volume at 10° C. or below and a second shape comprising a second volume at 29° C. or above, wherein the first volume is smaller than the second volume by at least 5%. In aspects of this embodiment, an expandable device disclosed herein having a first shape comprising a first volume at 10° C. or below and a second shape comprising a second volume at 30° C. or above, wherein the first volume is smaller than the second volume by at least 5%. In aspects of this embodiment, an expandable device disclosed herein having a first shape comprising a first volume at 10° C. or below and a second shape comprising a second volume at 32° C. or above, wherein the first volume is smaller than the second volume by at least 5%. In aspects of this embodiment, an expandable device disclosed herein having a first shape comprising a first volume at 10° C. or below and a second shape comprising a second volume at 34° C. or above, wherein the first volume is smaller than the second volume by at least 5%. In aspects of this embodiment, an expandable device disclosed herein having a first shape comprising a first volume at 10° C. or below and a second shape comprising a second volume at 35° C. or above, wherein the first volume is smaller than the second volume by at least 5%. In aspects of this embodiment, an expandable device disclosed herein having a first shape comprising a first volume at 10° C. or below and a second shape comprising a second volume at 36° C. or above, wherein the first volume is smaller than the second volume by at least 5%.

In aspects of this embodiment, an expandable device disclosed herein having a first shape comprising a first volume at 10° C. or below and a second shape comprising a second volume at 29° C. or above, wherein the second volume is larger than the first volume. In aspects of this embodiment, an expandable device disclosed herein having a first shape comprising a first volume at 10° C. or below and a second shape comprising a second volume at 30° C. or above, wherein the second volume is larger than the first volume. In aspects of this embodiment, an expandable device disclosed herein having a first shape comprising a first volume at 10° C. or below and a second shape comprising a second volume at 32° C. or above, wherein the second volume is larger than the first volume. In aspects of this embodiment, an expandable device disclosed herein having a first shape comprising a first volume at 10° C. or below and a second shape comprising a second volume at 34° C. or above, wherein the second volume is larger than the first volume. In aspects of this embodiment, an expandable device disclosed herein having a first shape comprising a first volume at 10° C. or below and a second shape comprising a second volume at 35° C. or above, wherein the second volume is larger than the first volume. In aspects of this embodiment, an expandable device disclosed herein having a first shape comprising a first volume at 10° C. or below and a second shape comprising a second volume at 36° C. or above, wherein the second volume is larger than the first volume.

In aspects of this embodiment, an expandable device disclosed herein having a first shape comprising a first volume at 10° C. or below and a second shape comprising a second volume at 29° C. or above, wherein the second volume is larger than the first volume by at least 5%. In aspects of this embodiment, an expandable device disclosed herein having a first shape comprising a first volume at 10° C. or below and a second shape comprising a second volume at 30° C. or above, wherein the second volume is larger than the first volume by at least 5%. In aspects of this embodiment, an expandable device disclosed herein having a first shape comprising a first volume at 10° C. or below and a second shape comprising a second volume at 32° C. or above, wherein the second volume is larger than the first volume by at least 5%. In aspects of this embodiment, an expandable device disclosed herein having a first shape comprising a first volume at 10° C. or below and a second shape comprising a second volume at 34° C. or above, wherein the second volume is larger than the first volume by at least 5%. In aspects of this embodiment, an expandable device disclosed herein having a first shape comprising a first volume at 10° C. or below and a second shape comprising a second volume at 35° C. or above, wherein the second volume is larger than the first volume by at least 5%. In aspects of this embodiment, an expandable device disclosed herein having a first shape comprising a first volume at 10° C. or below and a second shape comprising a second volume at 36° C. or above, wherein the second volume is larger than the first volume by at least 5%.

In one embodiment, an expandable device disclosed herein having a first shape comprising a first volume at body response temperature and a second shape comprising a second volume at a temperature above body response temperature, wherein the first volume is smaller than the second volume.

In aspects of this embodiment, an expandable device disclosed herein having a first shape comprising a first volume at 37° C. or below and a second shape comprising a second volume at 41° C. or above, wherein the first volume is smaller than the second volume. In aspects of this embodiment, an expandable device disclosed herein having a first shape comprising a first volume at 37° C. or below and a second shape comprising a second volume at 43° C. or above, wherein the first volume is smaller than the second volume. In aspects of this embodiment, an expandable device disclosed herein having a first shape comprising a first volume at 37° C. or below and a second shape comprising a second volume at 45° C. or above, wherein the first volume is smaller than the second volume. In aspects of this embodiment, an expandable device disclosed herein having a first shape comprising a first volume at 37° C. or below and a second shape comprising a second volume at 47° C. or above, wherein the first volume is smaller than the second volume. In aspects of this embodiment, an expandable device disclosed herein having a first shape comprising a first volume at 37° C. or below and a second shape comprising a second volume at 49° C. or above, wherein the first volume is smaller than the second volume.

In aspects of this embodiment, an expandable device disclosed herein having a first shape comprising a first volume at 38° C. or below and a second shape comprising a second volume at 42° C. or above, wherein the first volume is smaller than the second volume. In aspects of this embodiment, an expandable device disclosed herein having a first shape comprising a first volume at 38° C. or below and a second shape comprising a second volume at 44° C. or above, wherein the first volume is smaller than the second volume. In aspects of this embodiment, an expandable device disclosed herein having a first shape comprising a first volume at 38° C. or below and a second shape comprising a second volume at 46° C. or above, wherein the first volume is smaller than the second volume. In aspects of this embodiment, an expandable device disclosed herein having a first shape comprising a first volume at 38° C. or below and a second shape comprising a second volume at 48° C. or above, wherein the first volume is smaller than the second volume. In aspects of this embodiment, an expandable device disclosed herein having a first shape comprising a first volume at 38° C. or below and a second shape comprising a second volume at 50° C. or above, wherein the first volume is smaller than the second volume.

In aspects of this embodiment, an expandable device disclosed herein having a first shape comprising a first volume at 39° C. or below and a second shape comprising a second volume at 43° C. or above, wherein the first volume is smaller than the second volume. In aspects of this embodiment, an expandable device disclosed herein having a first shape comprising a first volume at 39° C. or below and a second shape comprising a second volume at 45° C. or above, wherein the first volume is smaller than the second volume. In aspects of this embodiment, an expandable device disclosed herein having a first shape comprising a first volume at 39° C. or below and a second shape comprising a second volume at 47° C. or above, wherein the first volume is smaller than the second volume. In aspects of this embodiment, an expandable device disclosed herein having a first shape comprising a first volume at 39° C. or below and a second shape comprising a second volume at 49° C. or above, wherein the first volume is smaller than the second volume.

In aspects of this embodiment, an expandable device disclosed herein having a first shape comprising a first volume at 40° C. or below and a second shape comprising a second volume at 44° C. or above, wherein the first volume is smaller than the second volume. In aspects of this embodiment, an expandable device disclosed herein having a first shape comprising a first volume at 40° C. or below and a second shape comprising a second volume at 46° C. or above, wherein the first volume is smaller than the second volume. In aspects of this embodiment, an expandable device disclosed herein having a first shape comprising a first volume at 40° C. or below and a second shape comprising a second volume at 48° C. or above, wherein the first volume is smaller than the second volume. In aspects of this embodiment, an expandable device disclosed herein having a first shape comprising a first volume at 40° C. or below and a second shape comprising a second volume at 50° C. or above, wherein the first volume is smaller than the second volume.

In aspects of this embodiment, an expandable device disclosed herein having a first shape comprising a first volume at 41° C. or below and a second shape comprising a second volume at 45° C. or above, wherein the first volume is smaller than the second volume. In aspects of this embodiment, an expandable device disclosed herein having a first shape comprising a first volume at 41° C. or below and a second shape comprising a second volume at 47° C. or above, wherein the first volume is smaller than the second volume. In aspects of this embodiment, an expandable device disclosed herein having a first shape comprising a first volume at 41° C. or below and a second shape comprising a second volume at 49° C. or above, wherein the first volume is smaller than the second volume.

In aspects of this embodiment, an expandable device disclosed herein having a first shape comprising a first volume at 37° C. or below and a second shape comprising a second volume at 41° C. or above, wherein the first volume is smaller than the second volume by at least 5%. In aspects of this embodiment, an expandable device disclosed herein having a first shape comprising a first volume at 37° C. or below and a second shape comprising a second volume at 43° C. or above, wherein the first volume is smaller than the second volume by at least 5%. In aspects of this embodiment, an expandable device disclosed herein having a first shape comprising a first volume at 37° C. or below and a second shape comprising a second volume at 45° C. or above, wherein the first volume is smaller than the second volume by at least 5%. In aspects of this embodiment, an expandable device disclosed herein having a first shape comprising a first volume at 37° C. or below and a second shape comprising a second volume at 47° C. or above, wherein the first volume is smaller than the second volume by at least 5%. In aspects of this embodiment, an expandable device disclosed herein having a first shape comprising a first volume at 37° C. or below and a second shape comprising a second volume at 49° C. or above, wherein the first volume is smaller than the second volume by at least 5%.

In aspects of this embodiment, an expandable device disclosed herein having a first shape comprising a first volume at 38° C. or below and a second shape comprising a second volume at 42° C. or above, wherein the first volume is smaller than the second volume by at least 5%. In aspects of this embodiment, an expandable device disclosed herein having a first shape comprising a first volume at 38° C. or below and a second shape comprising a second volume at 44° C. or above, wherein the first volume is smaller than the second volume by at least 5%. In aspects of this embodiment, an expandable device disclosed herein having a first shape comprising a first volume at 38° C. or below and a second shape comprising a second volume at 46° C. or above, wherein the first volume is smaller than the second volume by at least 5%. In aspects of this embodiment, an expandable device disclosed herein having a first shape comprising a first volume at 38° C. or below and a second shape comprising a second volume at 48° C. or above, wherein the first volume is smaller than the second volume by at least 5%. In aspects of this embodiment, an expandable device disclosed herein having a first shape comprising a first volume at 38° C. or below and a second shape comprising a second volume at 50° C. or above, wherein the first volume is smaller than the second volume by at least 5%.

In aspects of this embodiment, an expandable device disclosed herein having a first shape comprising a first volume at 39° C. or below and a second shape comprising a second volume at 43° C. or above, wherein the first volume is smaller than the second volume by at least 5%. In aspects of this embodiment, an expandable device disclosed herein having a first shape comprising a first volume at 39° C. or below and a second shape comprising a second volume at 45° C. or above, wherein the first volume is smaller than the second volume by at least 5%. In aspects of this embodiment, an expandable device disclosed herein having a first shape comprising a first volume at 39° C. or below and a second shape comprising a second volume at 47° C. or above, wherein the first volume is smaller than the second volume by at least 5%. In aspects of this embodiment, an expandable device disclosed herein having a first shape comprising a first volume at 39° C. or below and a second shape comprising a second volume at 49° C. or above, wherein the first volume is smaller than the second volume by at least 5%.

In aspects of this embodiment, an expandable device disclosed herein having a first shape comprising a first volume at 40° C. or below and a second shape comprising a second volume at 44° C. or above, wherein the first volume is smaller than the second volume by at least 5%. In aspects of this embodiment, an expandable device disclosed herein having a first shape comprising a first volume at 40° C. or below and a second shape comprising a second volume at 46° C. or above, wherein the first volume is smaller than the second volume by at least 5%. In aspects of this embodiment, an expandable device disclosed herein having a first shape comprising a first volume at 40° C. or below and a second shape comprising a second volume at 48° C. or above, wherein the first volume is smaller than the second volume by at least 5%. In aspects of this embodiment, an expandable device disclosed herein having a first shape comprising a first volume at 40° C. or below and a second shape comprising a second volume at 50° C. or above, wherein the first volume is smaller than the second volume by at least 5%.

In aspects of this embodiment, an expandable device disclosed herein having a first shape comprising a first volume at 41° C. or below and a second shape comprising a second volume at 45° C. or above, wherein the first volume is smaller than the second volume by at least 5%. In aspects of this embodiment, an expandable device disclosed herein having a first shape comprising a first volume at 41° C. or below and a second shape comprising a second volume at 47° C. or above, wherein the first volume is smaller than the second volume by at least 5%. In aspects of this embodiment, an expandable device disclosed herein having a first shape comprising a first volume at 41° C. or below and a second shape comprising a second volume at 49° C. or above, wherein the first volume is smaller than the second volume by at least 5%.

Aspects of the present specification disclose, in part, an expandable device disclosed herein that is composed of an expandable metal. An expandable metal disclosed herein includes a bimetal and a shape memory alloy. A bimetal is a laminate of two separate metals, and as such, is not an alloy, which is a mixture of two or more metals. Each metal layer of a bimetal has an unequal rate of thermal expansion due to different coefficients of thermal expansion. Upon a change in temperature, these unequal expansion rates will force the bimetal to bend one way if heated above its initial temperature, and in the opposite direction if cooled below its initial temperature. The metal layer with the higher coefficient of thermal expansion is on the outer side of the curve when the strip is heated and on the inner side when cooled. Thus, the different thermal expansion rates convert a temperature change into mechanical displacement.

Thermal expansion is the tendency of matter to change in volume in response to a change in temperature. When a substance is heated, its particles move faster and thus usually maintain an increased average separation. Conversely, when a substance is cooled, its particles move more slowly and thus usually maintain a decreased average separation. The rate of thermal expansion can be quantified by determining the degree of expansion divided by the change in temperature. Called the coefficient of thermal expansion, this value is calculated by measuring the fractional change in size of a material per degree change in temperature at a constant pressure. Several types of coefficients of thermal expansion have been developed including linear, volumetric, and area.

To a first approximation, the change in length of a metal due to thermal expansion is related to temperature change by a linear expansion coefficient α_{L}. It is the fractional change in length of the metal per degree of temperature change and can be calculated using the formula below:

$${α}_{L} = \frac{1}{{L}_{0}} \frac{Δ L}{Δ T} ,$$

where L₀ is a particular length measurement of the metal and ΔL/ΔT is the rate of change of that linear dimension per unit change in temperature, where ΔL=(L_{final}−Lᵢₙᵢₜᵢₐₗ) and ΔT=(T_{final}−Tᵢₙᵢₜᵢₐₗ). The change in the linear dimension can be estimated to be: ΔL=α_{L} Lₒ ΔT. A higher α_{L} means a larger ΔL for the same Lₒ and ΔT. This equation works well to estimate thermal expansion as long as the linear-expansion coefficient does not change much over the change in temperature ΔT. If it does, the equation must be integrated. Linear coefficient α_{L} at 20° C. for most materials is typically from 0.01×10⁻⁶/K to 350×10⁻⁶/K.

The volumetric thermal expansion coefficient is the most basic thermal expansion coefficient. In general, metals expand or contract when their temperature changes, with expansion or contraction occurring in all directions. Metals that expand at the same rate in every direction are called isotropic. For isotropic materials, the area and linear coefficients may be calculated from the volumetric coefficient. The volumetric coefficient of thermal expansion α_{V} is the fractional change in volume of a metal per degree of temperature change and can be calculated using the formula below:

$${α}_{V} = \frac{1}{{V}_{0}} \frac{Δ V}{Δ T} ,$$

where V₀ is a particular volumetric measurement of the metal and ΔV/ΔT is the rate of change of that volumetric dimension per unit change in temperature, where ΔV=(V_{final}−Vᵢₙᵢₜᵢₐₗ) and ΔT=(T_{final}−Tᵢₙᵢₜᵢₐₗ). These measurements are made at constant pressure p, although pressure does not appreciably affect the size of a metal, and as such, it is usually not necessary to specify that the pressure be held constant. The change in the volume can be estimated to be: ΔV=α_{V} V₀ ΔT. A higher α_{V} means a larger ΔV for the same Vₒ and ΔT. This equation works well to estimate thermal expansion as long as the volumetric expansion coefficient does not change much over the change in temperature ΔT. If it does, the equation must be integrated. Volumetric coefficient α_{V} at 20° C. for most materials is typically from 1×10⁻⁶/K to 1,000×10⁻⁶/K.

Bend curvature of a bimetal can be calculated using the formula below:

$$κ = \frac{6 {E}_{1} {E}_{2} ( {h}_{1} - {h}_{2} ) {h}_{1} {h}_{2} ε}{{E}_{1}^{2} {h}_{1}^{4} + 4 {E}_{1} {E}_{2} {h}_{1}^{3} {h}_{2} + 6 {E}_{1} {E}_{2} {h}_{1}^{2} {h}_{2}^{2} + 4 {E}_{1} {E}_{2} {h}_{2}^{3} {h}_{1} + {E}_{2}^{2} {h}_{2}^{4}} ,$$

where E₁ and h₁ are the Young's Modulus and height of the first metal and E₂ and h₂ are the Young's Modulus and height of the second metal. ε is the misfit strain, calculated by: ε=(α₁−α₂)ΔT, where α₁ is the Coefficient of Thermal Expansion of the first metal and α₂ is the Coefficient of Thermal Expansion of the second metal. ΔT is the current temperature minus the reference temperature (the temperature where the bimetal has no flexure).

Bimetals are made primarily by simultaneous rolling or pressing of two slabs of different metals or alloys. The casting of a fusible metal onto a refractory one and the immersion of a refractory metal in a molten fusible one are also common. In electroplating, the layer of the more precious metal is applied electrolytically. The relatively expensive and scarce harder alloys are applied to steel by electric heating.

In one embodiment, an expandable device disclosed herein is composed of a bimetal comprising an outer layer and an inner layer, wherein the outer layer and inner layer have unequal thermal expansion rates. In one embodiment, an expandable device disclosed herein is composed of a bimetal comprising an outer layer with a first thermal expansion rate and an inner layer with a second thermal expansion rate, wherein the outer layer and inner layer have unequal thermal expansion rates. In aspects of this embodiment, an expandable device disclosed herein is composed of a bimetal comprising an outer layer and an inner layer, wherein the outer layer has a higher coefficient of thermal expansion than the inner layer. In aspects of this embodiment, an expandable device disclosed herein is composed of a bimetal comprising an outer layer having a first coefficient of thermal expansion and an inner layer having a second coefficient of thermal expansion, wherein the first coefficient of thermal expansion is higher than the second coefficient of thermal expansion.

Each of the first and second metal layers of a bimetal can comprise a metal or a metal alloy. In one embodiment an outer layer and an inner layer are composed of a metal. In one embodiment an outer layer and an inner layer are composed of a metal alloy. In one embodiment an outer layer is composed of a metal and an inner layer is composed of a metal alloy. In one embodiment an outer layer is composed of a metal alloy and an inner layer is composed of a metal.

A metal may be composed of a transition metal, a poor metal or a metalloid group. In aspects of this embodiment, a metal may be comprised of Aluminum, Antimony, Arsenic, Bismuth, Bohrium, Boron, Cadmium, Chromium, Cobalt, Copernicium, Copper, Dubnium, Gallium, Germanium, Gold, Hafnium, Hassium, Indium, Iridium, Iron, Lead, Manganese, Mercury, Molybdenum, Niobium, Nickel, Osmium, Palladium, Platinum, Polonium, Rethenium, Rhenium, Rhodium, Rutherfordium, Scandium, Seaborgium, Silicon, Silver, Tantaium, Technetium, Tellurium, Thallium, Tin, Titanium, Tungsten, Vanadium, Yttrium, Zinc, and Zirconium, in any combination. In other aspects of this embodiment, a metal may be comprise of Aluminum, Cadmium, Chromium, Cobalt, Copper, Gallium, Germanium, Gold, Hafnium, Indium, Iron, Manganese, Nichol, Niobium, Palladium, Platinum, Tantalum, Scandium, Silicon, Silver, Tin, Titanium, Vanadium, Yttrium, Zinc, and Zirconium, in any combination. In yet other aspects of this embodiment, a metal may be comprise of Aluminum, Cadmium, Copper, Gold, Hafnium, Iron, Manganese, Nichol, Niobium, Palladium, Platinum, Silicon, Silver, Tin, Titanium, Zinc, and Zirconium, in any combination. In yet other aspects of this embodiment, a metal may be comprise of Aluminum, Cadmium, Copper, Gold, Hafnium, Iron, Manganese, Nichol, Niobium, Palladium, Silicon, Titanium, Zinc, and Zirconium, in any combination.

A metal may be a metal alloy composed of two or more elements belonging to the transition metal, poor metal, and metalloid groups. In aspects of this embodiment, a metal alloy may comprise two or more of Aluminum, Antimony, Arsenic, Bismuth, Bohrium, Boron, Cadmium, Chromium, Cobalt, Copernicium, Copper, Dubnium, Gallium, Germanium, Gold, Hafnium, Hassium, Indium, Iridium, Iron, Lead, Manganese, Mercury, Molybdenum, Niobium, Nickel, Osmium, Palladium, Platinum, Polonium, Rethenium, Rhenium, Rhodium, Rutherfordium, Scandium, Seaborgium, Silicon, Silver, Tantaium, Technetium, Tellurium, Thallium, Tin, Titanium, Tungsten, Vanadium, Yttrium, Zinc, and Zirconium, in any combination. In other aspects of this embodiment, a metal alloy may comprise two or more of Aluminum, Cadmium, Chromium, Cobalt, Copper, Gallium, Germanium, Gold, Hafnium, Indium, Iron, Manganese, Nichol, Niobium, Palladium, Platinum, Tantalum, Scandium, Silicon, Silver, Tin, Titanium, Vanadium, Yttrium, Zinc, and Zirconium, in any combination. In yet other aspects of this embodiment, a metal alloy may comprise two or more of Aluminum, Cadmium, Copper, Gold, Hafnium, Iron, Manganese, Nichol, Niobium, Palladium, Platinum, Silicon, Silver, Tin, Titanium, Zinc, and Zirconium, in any combination. In yet other aspects of this embodiment, a metal alloy may comprise two or more of Aluminum, Cadmium, Copper, Gold, Hafnium, Iron, Manganese, Nichol, Niobium, Palladium, Silicon, Titanium, Zinc, and Zirconium, in any combination.

In other aspects of this embodiment, a metal alloy may comprise Cobolt-Nichol-Aluminum (CoNiAl), Cobolt-Nichol-Gallium (CoNiGa), Copper-Tin (CuSn), Copper-Zinc (CuZn), Copper-Aluminum-Iron (CuAlFe), Copper-Aluminum-Nickel (CuAlNi), Copper-Zinc-Aluminum (CuZnAl), Copper-Zinc-Silicon (CuZnSi), Copper-Zinc-Tin (CuZnSn), Gold-Cadmium (AuCd), Hafnium-Titanium-Nickel (HfTiNi), Iron-Platinum (FePt), Iron-Manganese-Silicon (FeMnSi), Iron-Zinc-Copper-Aluminum (FeZnCuAl), Manganese-Copper (MnCu), Nickel-Iron-Gallium (NiFeGa), Nickel-Manganese-Gallium (NiMnGa), Nickel-Titanium (NiTi) (nitinol), Nickel-Titanium-Copper (NiTiCu), Nickel-Zirconium-Titanium (NiZrTi), Nickel-Iron-Zinc-Aluminum (NiFeZnAl), Silver-Cadmium (AgCd), Titanium-Palladium-Nickel (TiPdNi), Titanium-Niobium (TiNb), Titanium-Niobium-Aluminum (TiNbAl), Uranium-Niobium (Nb), or Zirconium-Copper-Zinc (ZrCuZn).

In one embodiment, an expandable device composed of a bimetal disclosed herein undergoes an unequal thermal expansion at or above a body response temperature. In aspects of this embodiment, an expandable device composed of a bimetal disclosed herein undergoes unequal thermal expansion at, e.g., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In other aspects of this embodiment, an expandable device composed of a bimetal disclosed herein undergoes unequal thermal expansion at, e.g., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., or at least 50° C. In yet other aspects of this embodiment, an expandable device composed of a bimetal disclosed herein undergoes unequal thermal expansion at, e.g., 24° C. or above, 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, 45° C. or above, 46° C. or above, 47° C. or above, 48° C. or above, 49° C. or above, or 50° C. or above.

In still other aspects of this embodiment, an expandable device composed of a bimetal disclosed herein undergoes unequal thermal expansion at, e.g., about 24° C. to about 26° C., about 24° C. to about 28° C., about 24° C. to about 30° C., about 24° C. to about 32° C., about 24° C. to about 34° C., about 24° C. to about 36° C., about 24° C. to about 38° C., about 24° C. to about 40° C., about 24° C. to about 42° C., about 24° C. to about 44° C., about 24° C. to about 46° C., about 24° C. to about 48° C., about 24° C. to about 50° C., about 25° C. to about 26° C., about 25° C. to about 28° C., about 25° C. to about 30° C., about 25° C. to about 32° C., about 25° C. to about 34° C., about 25° C. to about 36° C., about 25° C. to about 38° C., about 25° C. to about 40° C., about 25° C. to about 42° C., about 25° C. to about 44° C., about 25° C. to about 46° C., about 25° C. to about 48° C., about 25° C. to about 50° C., about 26° C. to about 28° C., about 26° C. to about 30° C., about 26° C. to about 32° C., about 26° C. to about 34° C., about 26° C. to about 36° C., about 26° C. to about 38° C., about 26° C. to about 40° C., about 26° C. to about 42° C., about 26° C. to about 44° C., about 26° C. to about 46° C., about 26° C. to about 48° C., about 26° C. to about 50° C., about 27° C. to about 28° C., about 27° C. to about 30° C., about 27° C. to about 32° C., about 27° C. to about 34° C., about 27° C. to about 36° C., about 27° C. to about 38° C., about 27° C. to about 40° C., about 27° C. to about 42° C., about 27° C. to about 44° C., about 27° C. to about 46° C., about 27° C. to about 48° C., about 27° C. to about 50° C., about 28° C. to about 30° C., about 28° C. to about 32° C., about 28° C. to about 34° C., about 28° C. to about 36° C., about 28° C. to about 38° C., about 28° C. to about 40° C., about 28° C. to about 42° C., about 28° C. to about 44° C., about 28° C. to about 46° C., about 28° C. to about 48° C., about 28° C. to about 50° C., about 29° C. to about 30° C., about 29° C. to about 32° C., about 29° C. to about 34° C., about 29° C. to about 36° C., about 29° C. to about 38° C., about 29° C. to about 40° C., about 29° C. to about 42° C., about 29° C. to about 44° C., about 29° C. to about 46° C., about 29° C. to about 48° C., about 29° C. to about 50° C., about 30° C. to about 32° C., about 30° C. to about 34° C., about 30° C. to about 36° C., about 30° C. to about 38° C., about 30° C. to about 40° C., about 30° C. to about 42° C., about 30° C. to about 44° C., about 30° C. to about 46° C., about 30° C. to about 48° C., about 30° C. to about 50° C., about 31° C. to about 32° C., about 31° C. to about 34° C., about 31° C. to about 36° C., about 31° C. to about 38° C., about 31° C. to about 40° C., about 31° C. to about 42° C., about 31° C. to about 44° C., about 31° C. to about 46° C., about 31° C. to about 48° C., about 31° C. to about 50° C., about 32° C. to about 34° C., about 32° C. to about 36° C., about 32° C. to about 38° C., about 32° C. to about 40° C., about 32° C. to about 42° C., about 32° C. to about 44° C., about 32° C. to about 46° C., about 32° C. to about 48° C., about 32° C. to about 50° C., about 33° C. to about 34° C., about 33° C. to about 36° C., about 33° C. to about 38° C., about 33° C. to about 40° C., about 33° C. to about 42° C., about 33° C. to about 44° C., about 33° C. to about 46° C., about 33° C. to about 48° C., about 33° C. to about 50° C., about 34° C. to about 36° C., about 34° C. to about 38° C., about 34° C. to about 40° C., about 34° C. to about 42° C., about 34° C. to about 44° C., about 34° C. to about 46° C., about 34° C. to about 48° C., about 34° C. to about 50° C., about 35° C. to about 36° C., about 35° C. to about 38° C., about 35° C. to about 40° C., about 35° C. to about 42° C., about 35° C. to about 44° C., about 35° C. to about 46° C., about 35° C. to about 48° C., about 35° C. to about 50° C., about 36° C. to about 38° C., about 36° C. to about 40° C., about 37° C. to about 38° C., about 37° C. to about 40° C., about 37° C. to about 42° C., about 37° C. to about 44° C., about 37° C. to about 46° C., about 37° C. to about 48° C., about 37° C. to about 50° C., about 38° C. to about 40° C., about 38° C. to about 42° C., about 38° C. to about 44° C., about 38° C. to about 46° C., about 38° C. to about 48° C., about 38° C. to about 50° C., about 39° C. to about 40° C., about 39° C. to about 42° C., about 39° C. to about 44° C., about 39° C. to about 46° C., about 39° C. to about 48° C., about 39° C. to about 50° C., about 40° C. to about 42° C., about 40° C. to about 44° C., about 40° C. to about 46° C., about 40° C. to about 48° C., about 40° C. to about 50° C., about 41° C. to about 42° C., about 41° C. to about 44° C., about 41° C. to about 46° C., about 41° C. to about 48° C., about 41° C. to about 50° C., about 42° C. to about 44° C., about 42° C. to about 46° C., about 42° C. to about 48° C., about 42° C. to about 50° C., about 43° C. to about 44° C., about 43° C. to about 46° C., about 43° C. to about 48° C., about 43° C. to about 50° C., about 44° C. to about 46° C., about 44° C. to about 48° C., about 44° C. to about 50° C., about 45° C. to about 46° C., about 45° C. to about 48° C., about 45° C. to about 50° C., about 46° C. to about 48° C., about 46° C. to about 50° C., about 47° C. to about 48° C., about 47° C. to about 50° C., or about 48° C. to about 50° C.

In one embodiment, an expandable device composed of a bimetal disclosed herein is of a thickness that is physiologically-acceptable and allows for a suitable degree of expansion at a body response temperature. In aspects of this embodiment, an expandable device composed of a bimetal herein has a thickness of, e.g., about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, or about 500 μm. In other aspects of this embodiment, an expandable device composed of a bimetal disclosed herein has a thickness of, e.g., at least 10 μm, at least 20 μm, at least 30 μm, at least 40 μm, at least 50 μm, at least 60 μm, at least 70 μm, at least 80 μm, at least 90 μm, at least 100 μm, at least 125 μm, at least 150 μm, at least 175 μm, at least 200 μm, at least 225 μm, at least 250 μm, at least 275 μm, at least 300 μm, at least 325 μm, at least 350 μm, at least 375 μm, at least 400 μm, at least 425 μm, at least 450 μm, at least 475 μm, or at least 500 μm. In yet other aspects of this embodiment, an expandable device composed of a bimetal disclosed herein has a thickness of, e.g., at most 10 μm, at most 20 μm, at most 30 μm, at most 40 μm, at most 50 μm, at most 60 μm, at most 70 μm, at most 80 μm, at most 90 μm, at most 100 μm, at most 125 μm, at most 150 μm, at most 175 μm, at most 200 μm, at most 225 μm, at most 250 μm, at most 275 μm, at most 300 μm, at most 325 μm, at most 350 μm, at most 375 μm, at most 400 μm, at most 425 μm, at most 450 μm, at most 475 μm, or at most 500 μm.

In still other aspects of this embodiment, an expandable device composed of a bimetal disclosed herein has a thickness of, e.g., about 10 μm to about 50 μm, about 10 μm to about 75 μm, about 10 μm to about 100 μm, about 10 μm to about 150 μm, about 10 μm to about 200 μm, about 10 μm to about 250 μm, about 10 μm to about 300 μm, about 10 μm to about 350 μm, about 10 μm to about 400 μm, about 10 μm to about 450 μm, about 10 μm to about 500 μm, about 25 μm to about 50 μm, about 25 μm to about 75 μm, about 25 μm to about 100 μm, about 25 μm to about 150 μm, about 25 μm to about 200 μm, about 25 μm to about 250 μm, about 25 μm to about 300 μm, about 25 μm to about 350 μm, about 25 μm to about 400 μm, about 25 μm to about 450 μm, about 25 μm to about 500 μm, about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, or about 250 μm to about 500 μm.

In one embodiment, an expandable device composed of a bimetal disclosed herein comprises an outer layer having a thickness that is physiologically-acceptable and allows for a suitable degree of expansion at a body response temperature. In aspects of this embodiment, an expandable device composed of a bimetal disclosed herein comprises an outer layer having a thickness of, e.g., about 0.1 μm, about 0.25 μm, about 0.5 μm, about 0.75 μm, about 1 μm, about 2.5 μm, about 5 μm, about 7.5 μm, about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, or about 500 μm. In other aspects of this embodiment, an expandable device composed of a bimetal disclosed herein comprises an outer layer having a thickness of, e.g., at least 0.1 μm, at least 0.25 μm, at least 0.5 μm, at least 0.75 μm, at least 1 μm, at least 2.5 μm, at least 5 μm, at least 7.5 μm, at least 10 μm, at least 20 μm, at least 30 μm, at least 40 μm, at least 50 μm, at least 60 μm, at least 70 μm, at least 80 μm, at least 90 μm, at least 100 μm, at least 125 μm, at least 150 μm, at least 175 μm, at least 200 μm, at least 225 μm, at least 250 μm, at least 275 μm, at least 300 μm, at least 325 μm, at least 350 μm, at least 375 μm, at least 400 μm, at least 425 μm, at least 450 μm, at least 475 μm, or at least 500 μm. In yet other aspects of this embodiment, an expandable device composed of a bimetal disclosed herein comprises an outer layer having a thickness of, e.g., at most 0.1 μm, at most 0.25 μm, at most 0.5 μm, at most 0.75 μm, at most 1 μm, at most 2.5 μm, at most 5 μm, at most 7.5 μm, at most 10 μm, at most 20 μm, at most 30 μm, at most 40 μm, at most 50 μm, at most 60 μm, at most 70 μm, at most 80 μm, at most 90 μm, at most 100 μm, at most 125 μm, at most 150 μm, at most 175 μm, at most 200 μm, at most 225 μm, at most 250 μm, at most 275 μm, at most 300 μm, at most 325 μm, at most 350 μm, at most 375 μm, at most 400 μm, at most 425 μm, at most 450 μm, at most 475 μm, or at most 500 μm.

In yet other aspects of this embodiment, an expandable device composed of a bimetal disclosed herein comprises an outer layer having a thickness of, e.g., about 0.1 μm to about 10 μm, about 0.1 μm to about 25 μm, about 0.1 μm to about 50 μm, about 0.1 μm to about 75 μm, about 0.1 μm to about 100 μm, about 0.1 μm to about 150 μm, about 0.1 μm to about 200 μm, about 0.1 μm to about 250 μm, about 0.1 μm to about 300 μm, about 0.1 μm to about 350 μm, about 0.1 μm to about 400 μm, about 0.1 μm to about 450 μm, about 0.1 μm to about 500 μm, about 1 μm to about 10 μm, about 1 μm to about 25 μm, about 1 μm to about 50 μm, about 1 μm to about 75 μm, about 1 μm to about 100 μm, about 1 μm to about 150 μm, about 1 μm to about 200 μm, about 1 μm to about 250 μm, about 1 μm to about 300 μm, about 1 μm to about 350 μm, about 1 μm to about 400 μm, about 1 μm to about 450 μm, about 1 μm to about 500 μm, about 5 μm to about 10 μm, about 5 μm to about 25 μm, about 5 μm to about 50 μm, about 5 μm to about 75 μm, about 5 μm to about 100 μm, about 5 μm to about 150 μm, about 5 μm to about 200 μm, about 5 μm to about 250 μm, about 5 μm to about 300 μm, about 5 μm to about 350 μm, about 5 μm to about 400 μm, about 5 μm to about 450 μm, about 5 μm to about 500 μm, about 10 μm to about 50 μm, about 10 μm to about 50 μm, about 10 μm to about 75 μm, about 10 μm to about 100 μm, about 10 μm to about 150 μm, about 10 μm to about 200 μm, about 10 μm to about 250 μm, about 10 μm to about 300 μm, about 10 μm to about 350 μm, about 10 μm to about 400 μm, about 10 μm to about 450 μm, about 10 μm to about 500 μm, about 25 μm to about 50 μm, about 25 μm to about 75 μm, about 25 μm to about 100 μm, about 25 μm to about 150 μm, about 25 μm to about 200 μm, about 25 μm to about 250 μm, about 25 μm to about 300 μm, about 25 μm to about 350 μm, about 25 μm to about 400 μm, about 25 μm to about 450 μm, about 25 μm to about 500 μm, about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, or about 250 μm to about 500 μm.

In one embodiment, an expandable device composed of a bimetal disclosed herein comprises an inner layer having a thickness that is physiologically-acceptable and allows for a suitable degree of expansion at a body response temperature. In aspects of this embodiment, an expandable device composed of a bimetal disclosed herein comprises an inner layer having a thickness of, e.g., about 0.1 μm, about 0.25 μm, about 0.5 μm, about 0.75 μm, about 1 μm, about 2.5 μm, about 5 μm, about 7.5 μm, about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, or about 500 μm. In other aspects of this embodiment, an expandable device composed of a bimetal disclosed herein comprises an inner layer having a thickness of, e.g., at least 0.1 μm, at least 0.25 μm, at least 0.5 μm, at least 0.75 μm, at least 1 μm, at least 2.5 μm, at least 5 μm, at least 7.5 μm, at least 10 μm, at least 20 μm, at least 30 μm, at least 40 μm, at least 50 μm, at least 60 μm, at least 70 μm, at least 80 μm, at least 90 μm, at least 100 μm, at least 125 μm, at least 150 μm, at least 175 μm, at least 200 μm, at least 225 μm, at least 250 μm, at least 275 μm, at least 300 μm, at least 325 μm, at least 350 μm, at least 375 μm, at least 400 μm, at least 425 μm, at least 450 μm, at least 475 μm, or at least 500 μm. In yet other aspects of this embodiment, an expandable device composed of a bimetal disclosed herein comprises an inner layer having a thickness of, e.g., at most 0.1 μm, at most 0.25 μm, at most 0.5 μm, at most 0.75 μm, at most 1 μm, at most 2.5 μm, at most 5 μm, at most 7.5 μm, at most 10 μm, at most 20 μm, at most 30 μm, at most 40 μm, at most 50 μm, at most 60 μm, at most 70 μm, at most 80 μm, at most 90 μm, at most 100 μm, at most 125 μm, at most 150 μm, at most 175 μm, at most 200 μm, at most 225 μm, at most 250 μm, at most 275 μm, at most 300 μm, at most 325 μm, at most 350 μm, at most 375 μm, at most 400 μm, at most 425 μm, at most 450 μm, at most 475 μm, or at most 500 μm.

In yet other aspects of this embodiment, an expandable device composed of a bimetal disclosed herein comprises an inner layer having a thickness of, e.g., about 0.1 μm to about 10 μm, about 0.1 μm to about 25 μm, about 0.1 μm to about 50 μm, about 0.1 μm to about 75 μm, about 0.1 μm to about 100 μm, about 0.1 μm to about 150 μm, about 0.1 μm to about 200 μm, about 0.1 μm to about 250 μm, about 0.1 μm to about 300 μm, about 0.1 μm to about 350 μm, about 0.1 μm to about 400 μm, about 0.1 μm to about 450 μm, about 0.1 μm to about 500 μm, about 1 μm to about 10 μm, about 1 μm to about 25 μm, about 1 μm to about 50 μm, about 1 μm to about 75 μm, about 1 μm to about 100 μm, about 1 μm to about 150 μm, about 1 μm to about 200 μm, about 1 μm to about 250 μm, about 1 μm to about 300 μm, about 1 μm to about 350 μm, about 1 μm to about 400 μm, about 1 μm to about 450 μm, about 1 μm to about 500 μm, about 5 μm to about 10 μm, about 5 μm to about 25 μm, about 5 μm to about 50 μm, about 5 μm to about 75 μm, about 5 μm to about 100 μm, about 5 μm to about 150 μm, about 5 μm to about 200 μm, about 5 μm to about 250 μm, about 5 μm to about 300 μm, about 5 μm to about 350 μm, about 5 μm to about 400 μm, about 5 μm to about 450 μm, about 5 μm to about 500 μm, about 10 μm to about 50 μm, about 10 μm to about 50 μm, about 10 μm to about 75 μm, about 10 μm to about 100 μm, about 10 μm to about 150 μm, about 10 μm to about 200 μm, about 10 μm to about 250 μm, about 10 μm to about 300 μm, about 10 μm to about 350 μm, about 10 μm to about 400 μm, about 10 μm to about 450 μm, about 10 μm to about 500 μm, about 25 μm to about 50 μm, about 25 μm to about 75 μm, about 25 μm to about 100 μm, about 25 μm to about 150 μm, about 25 μm to about 200 μm, about 25 μm to about 250 μm, about 25 μm to about 300 μm, about 25 μm to about 350 μm, about 25 μm to about 400 μm, about 25 μm to about 450 μm, about 25 μm to about 500 μm, about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, or about 250 μm to about 500 μm.

A shape memory alloy (SMA) is a mixture of two or more metallic compounds that demonstrate the ability to return to its original, cold-forged shape when subjected to the appropriate heating schedule. Also known as a smart metal, memory metal, memory alloy, muscle wire, or smart alloy, an SMA can undergo substantial plastic deformation and then be triggered into returning to its original pre-deformed shape by the application of heat. This thermally-induced elastic recovery that can be used to change an SMA with a small volume to a larger one.

An SMA has two phase changes as a solid which rearrange the positions of atoms within the crystal structure, known as the martensite (low-temperature) phase and the austenitic (high-temperature) phase. In a martensite phase, the arrangement of atoms are in a less compact and less organized pattern, forming a flexible, twinned crystal structure. An SMA in the martensite phase is relatively soft and easily deformed. In an austenite phase, the arrangement of atoms are in a more compact and organized pattern, resulting in a rigid, cubic crystal structure. An SMA in the austenitic phase is relatively strong and not easily deformed. Upon deformation this phase takes on the second form shown in figure above, on the right. Austenite, the stronger phase of shape memory alloys, occurs at higher temperatures. With no stress, an SMA is the same size and shape on a macroscopic scale under both phases, so that no change in size or shape is visible until a SMA is deformed in the martensite phase. The transition from the martensite phase to the austenite phase is only dependent on temperature and stress, not time.

The transition temperature is the temperature at which an SMA transition from the martensite phase to the austenite phase. Below the transition temperature, an SMA exists in the martensite phase. Above the transition temperature, an SMA reverts from the martensite to the austenite phase which changes it back into its parent shape.

The transition temperature of an SMA depends, in part, upon the type of metallic compounds used and the percentages of each of the metallic compounds present. These factors affect the temperatures at which the martensite and austenite phases begin and finish forming (Martensite start (Ms), Martensite finish (Mf), Austenite start (As), and Austenite finish (Af) temperatures). Ms is the temperature at which the transition to martensite starts upon cooling. Mf is the temperature at which the transition to martensite completes upon cooling. As is the temperature at which the transformation from martensite to austenite starts. Af is the temperature at which the transformation from martensite to austenite finishes.

The shape memory effect is observed when the temperature of a piece of shape memory alloy is cooled to below the temperature Mf. At this stage the alloy is completely in the martensite phase which can be easily deformed. After distorting the SMA the original shape can be recovered simply by heating the wire above the temperature Af. The heat transferred to the wire is the power driving the molecular rearrangement of the alloy, similar to heat melting ice into water, but the alloy remains solid. The deformed martensite is now transformed to the cubic austenite phase, which is configured in the original shape of the wire.

An SMA possesses two closely related and unique properties, shape memory effect and superelasticity (also known as pseudo-elasticity). Shape memory is the ability of an SMA to undergo deformation at one temperature, then recover its original, undeformed shape upon heating above its transition temperature (thermal-dependent elasticity). When an SMA is below its transition temperature (temperature is less than Af), it can be deformed and the alloy will hold that shape until heated above the transition temperature. Upon heating, the SMA recovers back to its original. When the metal cools again it will remain in its original, pre-deformed shape, until deformed again. Shape-memory alloys have different shape-memory effects, with the two most common being one-way shape memory effect and two-way shape memory effect. With the one-way effect, cooling from high temperatures does not cause a macroscopic shape change in an SMA. A deformation is necessary to create the low-temperature shape. On heating, transformation starts at As and is completed at Af. The two-way shape-memory effect is the effect that an SMA “remembers” two different shapes: one at low temperature, and another at the high-temperature.

Superelasticity is the ability of an SMA to undergo large deformations and then instantly revert to its original pre-deformed shape when the load is removed in the absence of any temperature change (thermal-independent elasticity). Superelasticity occurs at a narrow temperature range just above the transition temperature of the SMA (temperature is greater than Af), when the alloy is completely in the austenite phase. Simply by applying a load on an SMA causes a transition from the austenite to the martensitic phase, which enables the SMA to absorb the load. However, as soon as the loading is decreased the martensite phase begins to transform back to austenite phase and the SMA resumes its original shape. Since the temperature of the SMA is above Af, this shape transformation is independent of temperature.

An SMA may be composed of two or more elements belonging to the transition metal, poor metal, and metalloid groups. In aspects of this embodiment, an SMA may comprise two or more of Aluminum, Antimony, Arsenic, Bismuth, Bohrium, Boron, Cadmium, Chromium, Cobalt, Copernicium, Copper, Dubnium, Gallium, Germanium, Gold, Hafnium, Hassium, Indium, Iridium, Iron, Lead, Manganese, Mercury, Molybdenum, Niobium, Nickel, Osmium, Palladium, Platinum, Polonium, Rethenium, Rhenium, Rhodium, Rutherfordium, Scandium, Seaborgium, Silicon, Silver, Tantaium, Technetium, Tellurium, Thallium, Tin, Titanium, Tungsten, Vanadium, Yttrium, Zinc, and Zirconium, in any combination. In other aspects of this embodiment, an SMA may comprise two or more of Aluminum, Cadmium, Chromium, Cobalt, Copper, Gallium, Germanium, Gold, Hafnium, Indium, Iron, Manganese, Nichol, Niobium, Palladium, Platinum, Tantalum, Scandium, Silicon, Silver, Tin, Titanium, Vanadium, Yttrium, Zinc, and Zirconium, in any combination. In yet other aspects of this embodiment, an SMA may comprise two or more of Aluminum, Cadmium, Copper, Gold, Hafnium, Iron, Manganese, Nichol, Niobium, Palladium, Platinum, Silicon, Silver, Tin, Titanium, Zinc, and Zirconium, in any combination. In yet other aspects of this embodiment, an SMA may comprise two or more of Aluminum, Cadmium, Copper, Gold, Hafnium, Iron, Manganese, Nichol, Niobium, Palladium, Silicon, Titanium, Zinc, and Zirconium, in any combination.

In other aspects of this embodiment, an SMA comprises Cobolt-Nichol-Aluminum (CoNiAl), Cobolt-Nichol-Gallium (CoNiGa), Copper-Tin (CuSn), Copper-Zinc (CuZn), Copper-Aluminum-Iron (CuAlFe), Copper-Aluminum-Nickel (CuAlNi), Copper-Zinc-Aluminum (CuZnAl), Copper-Zinc-Silicon (CuZnSi), Copper-Zinc-Tin (CuZnSn), Gold-Cadmium (AuCd), Hafnium-Titanium-Nickel (HfTiNi), Iron-Platinum (FePt), Iron-Manganese-Silicon (FeMnSi), Iron-Zinc-Copper-Aluminum (FeZnCuAl), Manganese-Copper (MnCu), Nickel-Iron-Gallium (NiFeGa), Nickel-Manganese-Gallium (NiMnGa), Nickel-Titanium (NiTi), Nickel-Titanium-Copper (NiTiCu), Nickel-Zirconium-Titanium (NiZrTi), Nickel-Iron-Zinc-Aluminum (NiFeZnAl), Silver-Cadmium (AgCd), Titanium-Palladium-Nickel (TiPdNi), Titanium-Niobium (TiNb), Titanium-Niobium-Aluminum (TiNbAl), Uranium-Niobium (Nb), and Zirconium-Copper-Zinc (ZrCuZn).

In one embodiment, an expandable device disclosed herein is composed of a bimetal comprising an outer layer and an inner layer, wherein the outer layer and inner layer have unequal thermal expansion rates. In one embodiment, an expandable device disclosed herein is composed of a bimetal comprising an outer layer with a first thermal expansion rate and an inner layer with a second thermal expansion rate, wherein the outer layer and inner layer have unequal thermal expansion rates. In aspects of this embodiment, an expandable device composed of a bimetal comprising an outer layer and an inner layer, wherein the outer layer has a higher coefficient of thermal expansion than the inner layer. In aspects of this embodiment, an expandable device composed of a bimetal comprising an outer layer having a first coefficient of thermal expansion and an inner layer having a second coefficient of thermal expansion, wherein the first coefficient of thermal expansion is higher than the second coefficient of thermal expansion.

In one embodiment, an expandable device composed of a SMA disclosed herein has a transition temperature at a body response temperature. In aspects of this embodiment, an expandable device composed of an SMA disclosed herein has a transition temperature of, e.g., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In other aspects of this embodiment, an expandable device composed of an SMA disclosed herein has a transition temperature of, e.g., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., or at least 50° C. In yet other aspects of this embodiment, an expandable device composed of an SMA disclosed herein has a transition temperature of, e.g., 24° C. or above, 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, 45° C. or above, 46° C. or above, 47° C. or above, 48° C. or above, 49° C. or above, or 50° C. or above.

In still other aspects of this embodiment, an expandable device composed of a SMA disclosed herein has a transition temperature of, e.g., about 24° C. to about 26° C., about 24° C. to about 28° C., about 24° C. to about 30° C., about 24° C. to about 32° C., about 24° C. to about 34° C., about 24° C. to about 36° C., about 24° C. to about 38° C., about 24° C. to about 40° C., about 24° C. to about 42° C., about 24° C. to about 44° C., about 24° C. to about 46° C., about 24° C. to about 48° C., about 24° C. to about 50° C., about 25° C. to about 26° C., about 25° C. to about 28° C., about 25° C. to about 30° C., about 25° C. to about 32° C., about 25° C. to about 34° C., about 25° C. to about 36° C., about 25° C. to about 38° C., about 25° C. to about 40° C., about 25° C. to about 42° C., about 25° C. to about 44° C., about 25° C. to about 46° C., about 25° C. to about 48° C., about 25° C. to about 50° C., about 26° C. to about 28° C., about 26° C. to about 30° C., about 26° C. to about 32° C., about 26° C. to about 34° C., about 26° C. to about 36° C., about 26° C. to about 38° C., about 26° C. to about 40° C., about 26° C. to about 42° C., about 26° C. to about 44° C., about 26° C. to about 46° C., about 26° C. to about 48° C., about 26° C. to about 50° C., about 27° C. to about 28° C., about 27° C. to about 30° C., about 27° C. to about 32° C., about 27° C. to about 34° C., about 27° C. to about 36° C., about 27° C. to about 38° C., about 27° C. to about 40° C., about 27° C. to about 42° C., about 27° C. to about 44° C., about 27° C. to about 46° C., about 27° C. to about 48° C., about 27° C. to about 50° C., about 28° C. to about 30° C., about 28° C. to about 32° C., about 28° C. to about 34° C., about 28° C. to about 36° C., about 28° C. to about 38° C., about 28° C. to about 40° C., about 28° C. to about 42° C., about 28° C. to about 44° C., about 28° C. to about 46° C., about 28° C. to about 48° C., about 28° C. to about 50° C., about 29° C. to about 30° C., about 29° C. to about 32° C., about 29° C. to about 34° C., about 29° C. to about 36° C., about 29° C. to about 38° C., about 29° C. to about 40° C., about 29° C. to about 42° C., about 29° C. to about 44° C., about 29° C. to about 46° C., about 29° C. to about 48° C., about 29° C. to about 50° C., about 30° C. to about 32° C., about 30° C. to about 34° C., about 30° C. to about 36° C., about 30° C. to about 38° C., about 30° C. to about 40° C., about 30° C. to about 42° C., about 30° C. to about 44° C., about 30° C. to about 46° C., about 30° C. to about 48° C., about 30° C. to about 50° C., about 31° C. to about 32° C., about 31° C. to about 34° C., about 31° C. to about 36° C., about 31° C. to about 38° C., about 31° C. to about 40° C., about 31° C. to about 42° C., about 31° C. to about 44° C., about 31° C. to about 46° C., about 31° C. to about 48° C., about 31° C. to about 50° C., about 32° C. to about 34° C., about 32° C. to about 36° C., about 32° C. to about 38° C., about 32° C. to about 40° C., about 32° C. to about 42° C., about 32° C. to about 44° C., about 32° C. to about 46° C., about 32° C. to about 48° C., about 32° C. to about 50° C., about 33° C. to about 34° C., about 33° C. to about 36° C., about 33° C. to about 38° C., about 33° C. to about 40° C., about 33° C. to about 42° C., about 33° C. to about 44° C., about 33° C. to about 46° C., about 33° C. to about 48° C., about 33° C. to about 50° C., about 34° C. to about 36° C., about 34° C. to about 38° C., about 34° C. to about 40° C., about 34° C. to about 42° C., about 34° C. to about 44° C., about 34° C. to about 46° C., about 34° C. to about 48° C., about 34° C. to about 50° C., about 35° C. to about 36° C., about 35° C. to about 38° C., about 35° C. to about 40° C., about 35° C. to about 42° C., about 35° C. to about 44° C., about 35° C. to about 46° C., about 35° C. to about 48° C., about 35° C. to about 50° C., about 36° C. to about 38° C., about 36° C. to about 40° C., about 37° C. to about 38° C., about 37° C. to about 40° C., about 37° C. to about 42° C., about 37° C. to about 44° C., about 37° C. to about 46° C., about 37° C. to about 48° C., about 37° C. to about 50° C., about 38° C. to about 40° C., about 38° C. to about 42° C., about 38° C. to about 44° C., about 38° C. to about 46° C., about 38° C. to about 48° C., about 38° C. to about 50° C., about 39° C. to about 40° C., about 39° C. to about 42° C., about 39° C. to about 44° C., about 39° C. to about 46° C., about 39° C. to about 48° C., about 39° C. to about 50° C., about 40° C. to about 42° C., about 40° C. to about 44° C., about 40° C. to about 46° C., about 40° C. to about 48° C., about 40° C. to about 50° C., about 41° C. to about 42° C., about 41° C. to about 44° C., about 41° C. to about 46° C., about 41° C. to about 48° C., about 41° C. to about 50° C., about 42° C. to about 44° C., about 42° C. to about 46° C., about 42° C. to about 48° C., about 42° C. to about 50° C., about 43° C. to about 44° C., about 43° C. to about 46° C., about 43° C. to about 48° C., about 43° C. to about 50° C., about 44° C. to about 46° C., about 44° C. to about 48° C., about 44° C. to about 50° C., about 45° C. to about 46° C., about 45° C. to about 48° C., about 45° C. to about 50° C., about 46° C. to about 48° C., about 46° C. to about 50° C., about 47° C. to about 48° C., about 47° C. to about 50° C., or about 48° C. to about 50° C.

In one embodiment, an expandable device composed of an SMA disclosed herein is of a thickness that is physiologically-acceptable and allows for a suitable degree of expansion at a body response temperature. In aspects of this embodiment, an expandable device composed of an SMA herein has a thickness of, e.g., about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, or about 500 μm. In other aspects of this embodiment, an expandable device composed of an SMA disclosed herein has a thickness of, e.g., at least 10 μm, at least 20 μm, at least 30 μm, at least 40 μm, at least 50 μm, at least 60 μm, at least 70 μm, at least 80 μm, at least 90 μm, at least 100 μm, at least 125 μm, at least 150 μm, at least 175 μm, at least 200 μm, at least 225 μm, at least 250 μm, at least 275 μm, at least 300 μm, at least 325 μm, at least 350 μm, at least 375 μm, at least 400 μm, at least 425 μm, at least 450 μm, at least 475 μm, or at least 500 μm. In yet other aspects of this embodiment, an expandable device composed of an SMA disclosed herein has a thickness of, e.g., at most 10 μm, at most 20 μm, at most 30 μm, at most 40 μm, at most 50 μm, at most 60 μm, at most 70 μm, at most 80 μm, at most 90 μm, at most 100 μm, at most 125 μm, at most 150 μm, at most 175 μm, at most 200 μm, at most 225 μm, at most 250 μm, at most 275 μm, at most 300 μm, at most 325 μm, at most 350 μm, at most 375 μm, at most 400 μm, at most 425 μm, at most 450 μm, at most 475 μm, or at most 500 μm.

In still other aspects of this embodiment, an expandable device composed of an SMA disclosed herein has a thickness of, e.g., about 10 μm to about 50 μm, about 10 μm to about 75 μm, about 10 μm to about 100 μm, about 10 μm to about 150 μm, about 10 μm to about 200 μm, about 10 μm to about 250 μm, about 10 μm to about 300 μm, about 10 μm to about 350 μm, about 10 μm to about 400 μm, about 10 μm to about 450 μm, about 10 μm to about 500 μm, about 25 μm to about 50 μm, about 25 μm to about 75 μm, about 25 μm to about 100 μm, about 25 μm to about 150 μm, about 25 μm to about 200 μm, about 25 μm to about 250 μm, about 25 μm to about 300 μm, about 25 μm to about 350 μm, about 25 μm to about 400 μm, about 25 μm to about 450 μm, about 25 μm to about 500 μm, about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, or about 250 μm to about 500 μm.

Aspects of the present specification disclose, in part, methods of employing an expandable device disclosed herein. In one embodiment, a method of employing an expandable device disclosed herein comprises the steps of a) inserting one end of an expandable device disclosed herein into a first lumen of a first vessel; b) inserting the other end of an expandable device disclosed herein into a second lumen of a second vessel; and c) enlarging the expandable device. In aspects of this method, the method may further include the step of attaching a fluid-filled device to a port on the expandable device. In aspects of this method, the method may further include the step of delivering fluid through a port on the expandable device.

Aspects of the present specification disclose, in part, methods of employing an expandable device disclosed herein. In one embodiment, a method of employing an expandable device disclosed herein comprises the steps of a) inserting an end of a first vessel into a first end of an expandable device disclosed herein; b) inserting an end of a second vessel into an opposite second end of an expandable device disclosed herein; and c) contracting the expandable device. In aspects of this method, the method may further include the step of attaching a fluid-filled device to a port on the expandable device. In aspects of this method, the method may further include the step of delivering fluid through a port on the expandable device. In aspects of this method, the method may further include the step of heating the expandable device to a temperature above body response temperature so as to expand the expandable device prior to insertion of the vessel therein.

Aspects of the present specification disclose, in part, use of an expandable device disclosed herein to create a connection between two or more tubular structures.

A first vessel and a second vessel disclosed herein may be an artery, an arteriole, a vein, a veiniole, a lymph vessel, a lymph node, or a gastrointestinal vessel such as, e.g., an esophagus, a small intestine, a large intestine.

A method or use disclosed herein may further comprise the step of applying suction to the inserted expandable device. The suction helps draw the ends of the two vessels closer together to form better anastomosis. Typically a vacuum pump is employed to apply the suction and a hose is attached to a port on an expandable device disclosed herein.

A method or use disclosed herein may further comprise the step of placing a portion of tunia externa from a vessel disclosed herein over a port of an expandable device disclosed herein in order to prevent vessel retraction and thus provide a more secure attachment. The portion of tunia externa may be placed over a port in such a manner that the port may continue to be used after such placement.

An expandable device disclosed herein may be enlarged by 1) allowing the device to be warmed by the body temperature of an individual emanating from the surrounding tissue, 2) injecting a fluid into the expandable device that is sufficiently warm enough to enlarge the device; 3) using a heating device that warms the expandable device to a temperature sufficiently warm enough to enlarge the device, or any combination thereof.

A method or use disclosed herein may be used to create a connection between two or more tubular structures. A tubular structure includes a vessel, such as, e.g., a circulatory vessel like a blood vessel and a lymphatic vessel, a neuronal vessel; a gastrointestinal vessel, such as, e.g., a small intestine, like a duodenum, a jujenum, or an illium; a large intestine, like a colon or a rectum, or a bile duct; an urogenital vessel, such as, e.g., a ureter, a urethra, a fallopian tube, or a vas deference vessel; an endocrine vessel such as, e.g., a pancreatic duct, a thyroid dust, an adrenal gland duct, or a pituitary duct; or a respiratory vessel such as, e.g., a bronchus or broonchiole. In one embodiment, a method or use disclosed herein may be used to create an end-to-end anastomosis, an end-to-side anastomosis, or a side-to-side anastomosis. In one embodiment, through a method or use disclosed herein a connection created is an anastomosis. In aspects of this embodiment, a method or use disclosed herein creates an arterio-arterial anastomosis, a veno-venous anastomosis, a lympho-lymphatic anastomosis, an arterio-venous anastomosis, a lympho-venous anastomosis, or a lymph node to a vein anastomosis. In other aspects of this embodiment, a method or use disclosed herein creates a gastrointestinal anastomosis. In yet other aspects of this embodiment, a method or use disclosed herein creates a urogenital anastomosis. In still other aspects of this embodiment, a method or use disclosed herein creates an endocrine anastomosis. In other aspects of this embodiment, a method or use disclosed herein creates a respiratory anastomosis.

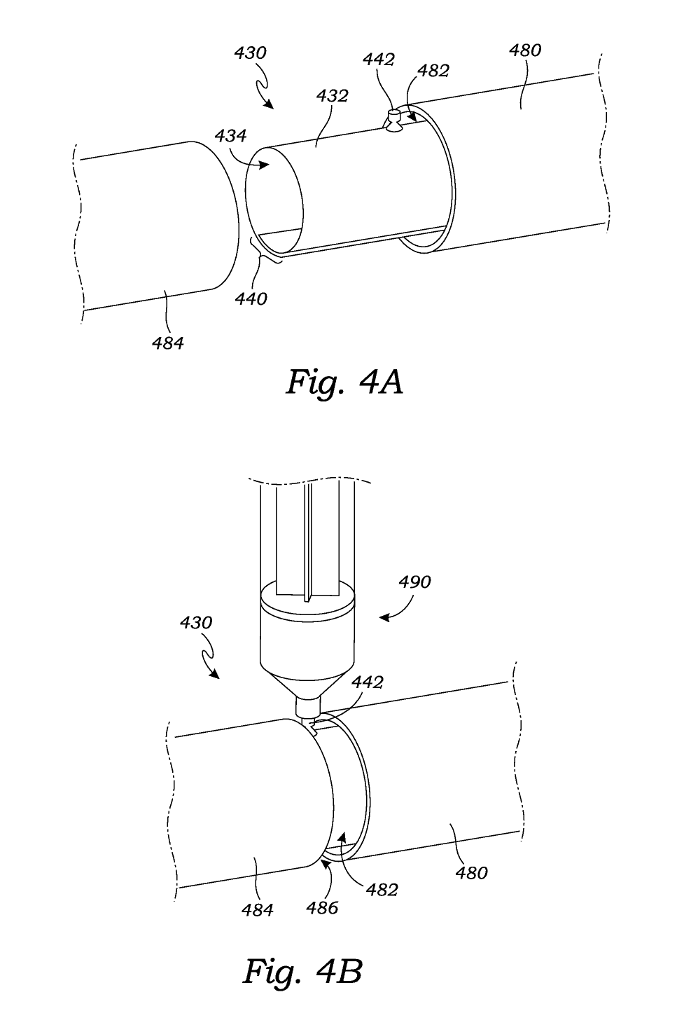

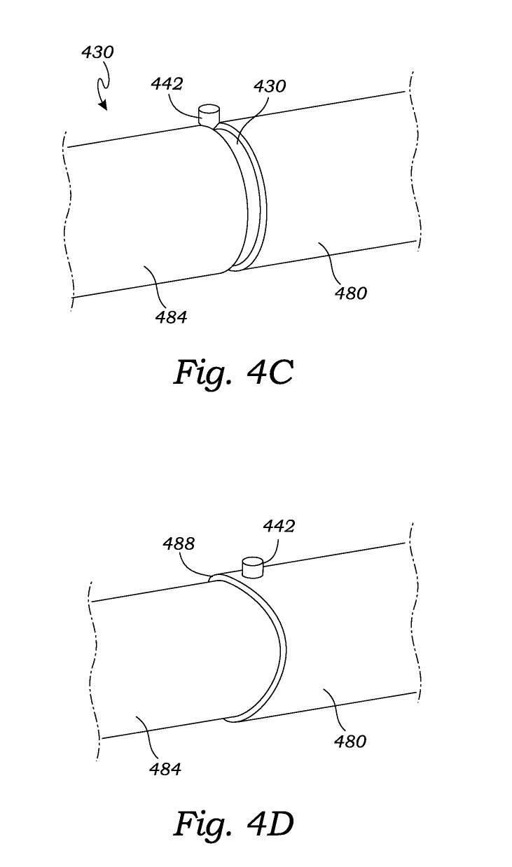

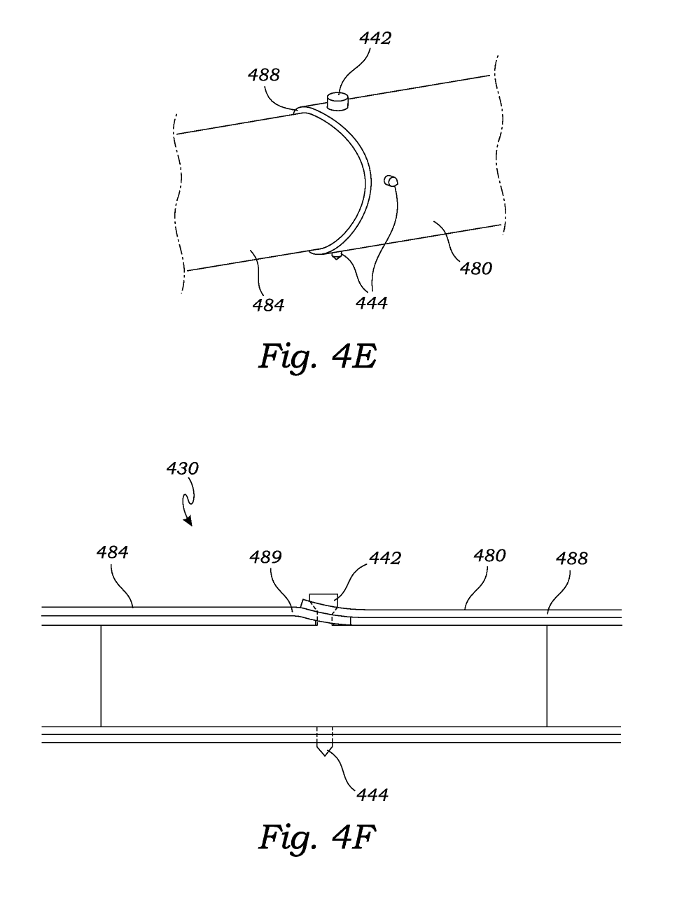

FIG. 4 illustrates one embodiment of a method or use disclosed herein using an expandable device comprising a tube assembly disclosed herein designed in an unsealed configuration. FIG. 4A shows expandable device 430 comprising split tube body 432, overlap region 440, lumen 434, and port 442, in a first shape comprising a first volume inserted in first lumen 482 of first vessel 480. FIG. 4B shows expandable device 430 still in its first shape comprising first volume inserted in first lumen 482 of first vessel 480 and in second lumen 486 of second vessel 484 and having fluid dispensing device 490 attached to port 442. FIG. 4C shows expandable device 430 in an expanded second shape comprising a second volume enlarged to fit substantially tightly in first vessel 480 and second vessel 484. In the second shape comprising the second volume, expandable device 430 has enlarged to such an extent that first lumen 482 of first vessel 480 and second lumen 486 of second vessel 484 have been occupied by expandable device 430. FIG. 4D shows a portion of tunica externa 488 of first vessel 480 and a portion of tunica externa 489 of second vessel 484 pulled over port 442. FIG. 4E shows a portion of tunica externa 488 of first vessel 480 and a portion of tunica externa 489 of second vessel 484 pulled over port 442 and spikes 444. FIG. 4F shows a portion of tunica externa 488 of first vessel 480 and a portion of tunica externa 489 of second vessel 484 pulled over port 442 and spike 444.

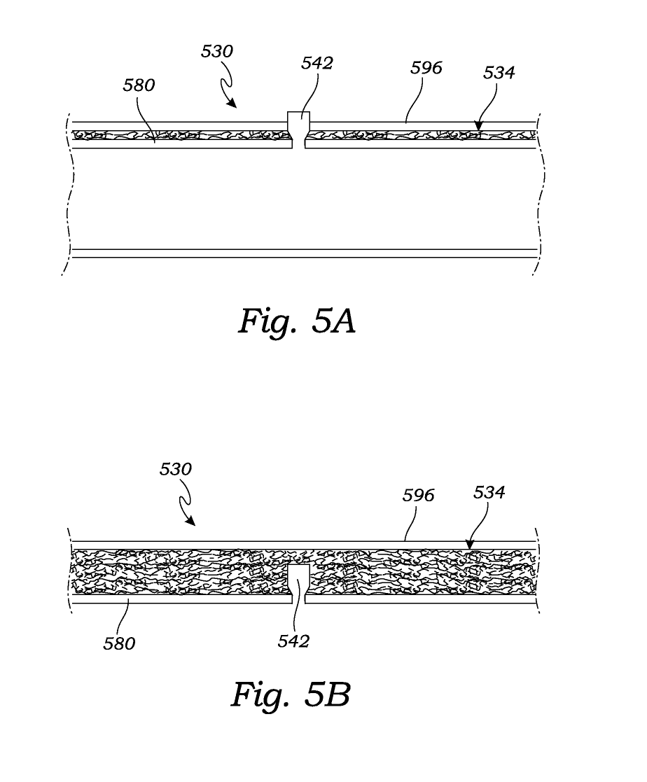

In one embodiment, a port disclosed herein is designed to extend through the skin and be accessible from outside the body. In one embodiment, a port disclosed herein is designed not to extend through the skin, but can be accessible from outside the body with the use of a catheter. FIG. 5 illustrates one embodiment. FIG. 5A shows implantation of expandable device 530 in vessel 580 having port 542 extending through skin surface 596, allowing port 542 to be accessible from outside the body. FIG. 5B shows implantation of expandable device 530 in vessel 580 having port 542 beneath skin surface 596, not allowing port 542 to be accessible from outside the body, at least without the use of some additional device such as a catheter.

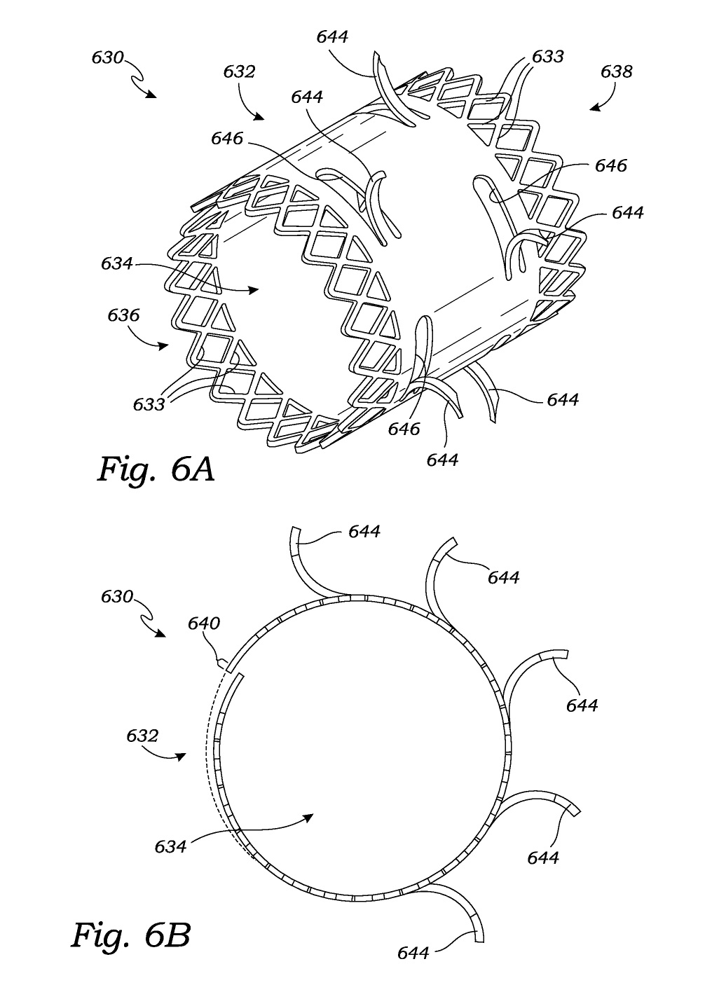

FIG. 6 illustrates a further embodiment of an expandable device comprising a tube assembly disclosed herein designed in an unsealed configuration. As shown in the perspective view of FIG. 6A, the expandable device 630 includes a split tube body 632 and lumen 634 defining a first luminal opening 636 and an opposite second luminal opening 638. The split tube body 632 is characterized in that at least at its opposite ends adjacent the first and second luminal openings 636, 638 there is formed a mesh pattern defined by mesh openings 633. It will be appreciated that such mesh openings 633 may assist in the retention of the expandable device 630 in or on a vessel, as by allowing the vessel wall itself—the tunica intima when the device is inserted inside a vessel or the tunica externa if the device is installed outside or around a vessel—to partially seat within or adhere to the tube body 632. While a particular pattern and geometrical configuration of the mesh openings 633 is shown, such is merely illustrative. As also shown in FIG. 6A, the central portion of the split tube body 632, between the opposite mesh portions, may be formed with one or more spikes 644 to further assist in securing the vessel relative to the expandable device 630. As illustrated, the exemplary spikes 644 are formed as tabs bent or curved radially outwardly from respective cut-outs 646 in the body 632. It will be appreciated that as shown with the spikes 644 formed so as to bend or curve radially outwardly they would pierce and seat within the vessel wall when the expandable device 630 is inserted inside a vessel or other tubular structure. Or, when the expandable device 630 is installed on the outside of a vessel, the spikes 644 may instead secure the position of the device 630 and vessel relative to the surrounding interstitial tissues as by piercing or engaging therewith. Alternatively, some or all of the spikes 644 may instead be bent or curved somewhat radially inwardly so as to then pierce and secure to a vessel placed within the expandable device 630; with some curved radially outwardly and some curved radially inwardly the device 630 may thus be anchored in or secured relative to both the interior vessel and the surrounding interstitial tissues. FIG. 6B shows an end view of expandable device 630, including split tube body 632 with spikes 644, overlap region 640, and lumen 634.

In aspects of an expandable device disclosed herein, the device may be secured to either or both the vessel or other tubular structure and/or surrounding interstitial tissue using any structure, mechanical means, or method now known or later developed, including but not limited to one or more spikes, one or more ports, one or more staples, and bio-compatible glue, alone or in any combination.

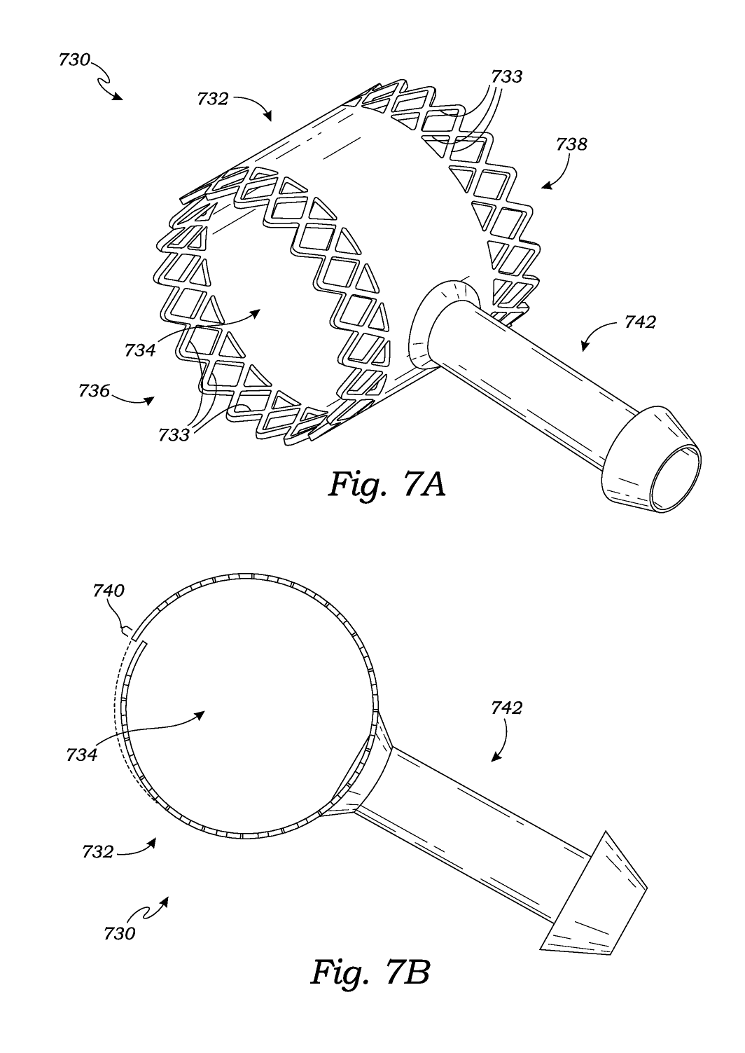

FIG. 7 illustrates a still further embodiment of an expandable device comprising a tube assembly disclosed herein designed in an unsealed configuration. As shown in the perspective view of FIG. 7A, the expandable device 730 includes a split tube body 732 and lumen 734 defining a first luminal opening 736 and an opposite second luminal opening 738. The split tube body 732 is characterized in that at least at its opposite ends adjacent the first and second luminal openings 736, 738 there is formed a mesh pattern defined by mesh openings 733. While a particular pattern and geometrical configuration of the mesh openings 733 is shown, such is merely illustrative. As also shown in FIG. 7A, the central portion of the split tube body 732, between the opposite mesh portions, may be formed with one or more port 742 to allow communication with the interior lumen 734 after the device 730 is installed. It will be appreciated that to the extent the port 742 passes through the vessel wall, the port 742 will also further assist in securing the vessel relative to the expandable device 730. FIG. 7B shows an end view of expandable device 730, including split tube body 732 with port 742, overlap region 740, and lumen 734.

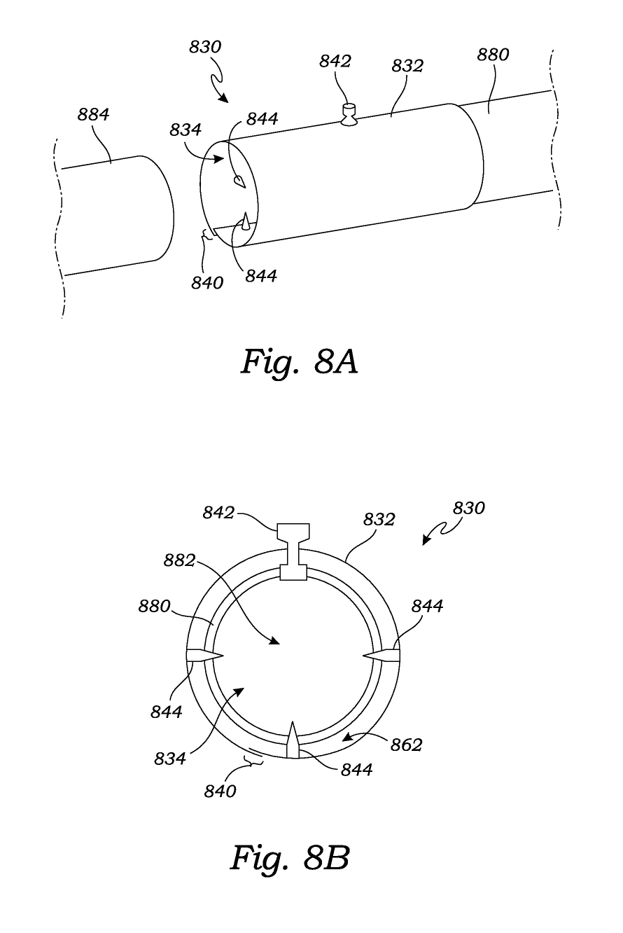

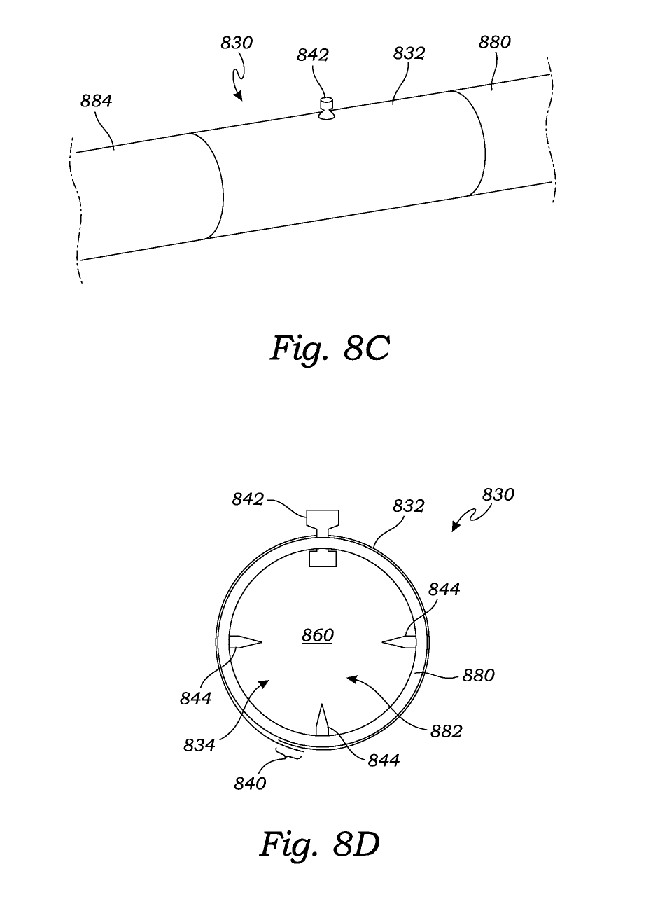

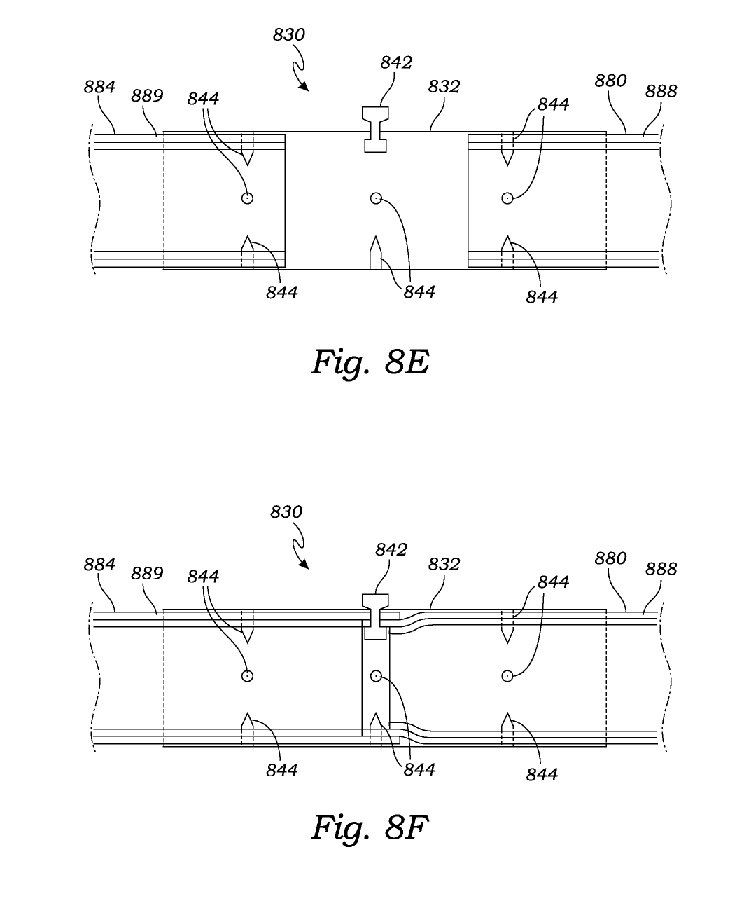

FIG. 8 illustrates a further embodiment of an expandable device comprising a tube assembly disclosed herein designed in an unsealed configuration. More particularly, the alternative exemplary embodiment is configured for placement outside of or around a vessel as herein described. As shown in the perspective view of FIG. 8A, the expandable device 830 includes split tube body 832, overlap region 840, lumen 834, port 842, and spikes 844, starting in an expanded second shape with a first vessel 880 inserted therein at one end. The spikes 844 are configured to project substantially radially inwardly, as best seen in FIG. 8B, so as to help secure any vessel or other tubular structure within the expandable device 830. FIG. 8B again shows an end sectional schematic view of expandable device 830, including split tube body 832 with spikes 844, overlap region 840, and lumen 834. Expandable device 830 is still in its expanded second shape comprising a second volume 862 and positioned over or about first vessel 880 having first lumen 882, with the spikes 844 having just pierced the vessel wall; in an alternative embodiment the spikes 844 may be configured such that with the device 830 in its expanded second shape the spikes 844 do not yet pierce the vessel 880. Likewise, while the port 842 is shown as having also pierced or passed through or been positioned in the wall of the vessel 880 that is not necessarily the case with the device 830 in its expanded second shape. Notably, the second volume 862 defined by the lumen 834 of the split tube body 832 of the expandable device 830 in its expanded second shape is thus greater than the volume occupied by the vessel 880 and its lumen 882, as shown schematically. FIG. 4C shows in perspective view expandable device 830 now in a contracted or “at rest” first shape secured about opposite first and second vessels 880, 884 so as to provide anastomosis thereto. FIG. 8D shows in an end schematic view expandable device 830 still in its relatively smaller or contracted first shape effectively in a net fit engagement about the vessel 880. Thus, the first volume 860 associated with the contracted expandable device 830, or the lumen 834 of the split tube body 832 in the first shape, substantially approximates the volume occupied by the vessel 880, the device 830 having contracted from its previously expanded state to fit substantially tightly about the first and second vessels 880, 884. FIG. 8E shows in one exemplary embodiment first and second vessels 880, 884 pulled over spikes 844 positioned adjacent opposite ends of the tubular body 832, with the central port 842 and any central spikes 844 not being engaged by either vessel 880, 884. FIG. 8F shows the respective first and second vessels 880, 884 inserted more fully, with a portion of tunica externa 888 of first vessel 880 and a portion of tunica externa 889 of second vessel 884 pulled over port 842 and over the one or more central spikes 844 as well.

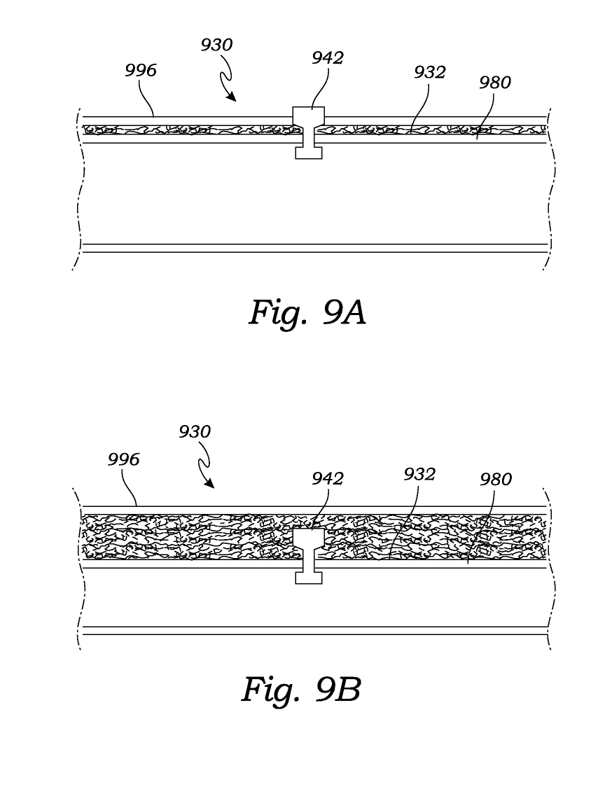

In one embodiment, a port disclosed herein is designed to extend through the skin and be accessible from outside the body. In one embodiment, a port disclosed herein is designed not to extend through the skin, but can be accessible from outside the body with the use of a catheter. FIG. 9 illustrates one embodiment. FIG. 9A shows installation of expandable device 930 on vessel 980 with the split tube body 932 thereabout and having port 942 extending through skin surface 996, allowing port 942 to be accessible from outside the body. FIG. 9B shows installation of expandable device 930 on vessel 980 with the split tube body 932 thereabout and having port 942 beneath skin surface 996, not allowing port 942 to be accessible from outside the body, at least without the use of some additional device such as a catheter.

Aspects of the present specification disclose, in part, kits comprising one or more expandable device disclosed herein. In one embodiment, a kit disclosed herein comprises a single expandable device disclosed herein. In one embodiment, a kit disclosed herein comprises a plurality of expandable devices disclosed herein. In aspects of this embodiment, a kit disclosed herein comprises, e.g., 1, 2, 3, 4, 5, 6, 7, 8, 9, 10, 11, 12, 13, 14, 15, 20, 25, 30, 35, 40, 45, or 50 expandable devices. In other aspects of this embodiment, a kit disclosed herein comprises, e.g., at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, or at least 50 expandable devices. In yet other aspects of this embodiment, a kit disclosed herein comprises, e.g., about 1 to about 5, about 1 to about 10, about 1 to about 15, about 1 to about 20, about 1 to about 25, about 1 to about 30, about 1 to about 35, about 1 to about 40, about 1 to about 45, about 1 to about 50, about 5 to about 10, about 5 to about 15, about 5 to about 20, about 5 to about 25, about 5 to about 30, about 5 to about 35, about 5 to about 40, about 5 to about 45, about 5 to about 50, about 10 to about 15, about 10 to about 20, about 10 to about 25, about 10 to about 30, about 10 to about 35, about 10 to about 40, about 10 to about 45, about 10 to about 50, about 15 to about 20, about 15 to about 25, about 15 to about 30, about 15 to about 35, about 15 to about 40, about 15 to about 45, about 15 to about 50, about 20 to about 25, about 20 to about 30, about 20 to about 35, about 20 to about 40, about 20 to about 45, about 20 to about 50, about 25 to about 30, about 25 to about 35, about 25 to about 40, about 25 to about 45, about 25 to about 50, about 30 to about 35, about 30 to about 40, about 30 to about 45, about 30 to about 50, about 35 to about 40, about 35 to about 45, about 35 to about 50, about 40 to about 45, about 40 to about 50, or about 45 to about 50 expandable devices.

A kit disclosed herein may comprise one or more expandable device disclosed herein and one or more containers comprising a fluid used to enlarge an expandable device. Typically, a fluid disclosed herein is a physiological-acceptable fluid. A physiological-acceptable fluid is a fluid that is nontoxic at the volumes and concentrations employed, and has substantially no long-term or permanent detrimental effect when administered to an individual. In aspects of this embodiment, a physiological-acceptable fluid is water, saline, or other buffered solution.

In one embodiment, a kit disclosed herein comprises a single container comprising a fluid used to enlarge an expandable device. In one embodiment, a kit disclosed herein comprises a plurality of containers comprising a fluid used to enlarge an expandable device. In aspects of this embodiment, a kit disclosed herein comprises, e.g., 1, 2, 3, 4, 5, 6, 7, 8, 9, 10, 11, 12, 13, 14, 15, 20, 25, 30, 35, 40, 45, or 50 containers comprising a fluid used to enlarge an expandable device. In other aspects of this embodiment, a kit disclosed herein comprises, e.g., at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, or at least 50 containers comprising a fluid used to enlarge an expandable device. In yet other aspect of this embodiment, a kit disclosed herein comprises, e.g., about 1 to about 5, about 1 to about 10, about 1 to about 15, about 1 to about 20, about 1 to about 25, about 1 to about 30, about 1 to about 35, about 1 to about 40, about 1 to about 45, about 1 to about 50, about 5 to about 10, about 5 to about 15, about 5 to about 20, about 5 to about 25, about 5 to about 30, about 5 to about 35, about 5 to about 40, about 5 to about 45, about 5 to about 50, about 10 to about 15, about 10 to about 20, about 10 to about 25, about 10 to about 30, about 10 to about 35, about 10 to about 40, about 10 to about 45, about 10 to about 50, about 15 to about 20, about 15 to about 25, about 15 to about 30, about 15 to about 35, about 15 to about 40, about 15 to about 45, about 15 to about 50, about 20 to about 25, about 20 to about 30, about 20 to about 35, about 20 to about 40, about 20 to about 45, about 20 to about 50, about 25 to about 30, about 25 to about 35, about 25 to about 40, about 25 to about 45, about 25 to about 50, about 30 to about 35, about 30 to about 40, about 30 to about 45, about 30 to about 50, about 35 to about 40, about 35 to about 45, about 35 to about 50, about 40 to about 45, about 40 to about 50, or about 45 to about 50 containers comprising a fluid used to enlarge an expandable device.

A kit disclosed herein may further comprise a heating device used to warm an expandable device disclosed herein in order to enlarge the device prior to or during implantation. Such a device can simply radiate heat or expel heat from the device using blown air.

A kit disclosed herein may include labels or inserts. Labels or inserts include “printed matter,” e.g., paper or cardboard, or separate or affixed to a component, a kit or packing material (e.g., a box), or attached to kit component. Labels or inserts can additionally include a computer readable medium, such as a disk (e.g., hard disk, flash memory), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts may include identifying information of one or more components therein, technical information of one or more components therein, instructions on how to use one or more components therein including a method or use disclosed herein, safety information on one or more components therein, information on potential adverse side effects of one or more components therein, complications, reactions, and/or warnings to the individual or clinician of one or more components therein and their use, manufacturer information identifying lot numbers, manufacturer location, and/or manufacturer date of kit and one or more components therein.

Aspects of the present specification may also be described as follows:
1. An expandable device comprising a tube assembly composed of an expandable metal, the tube assembly comprising a tube body having a lumen defining a first luminal opening and a second luminal opening, wherein the tube body forms the lumen and the first and second luminal openings are at each end of the tube assembly, and wherein the expandable device is configured to exist in at least two different shapes each comprising a volume, the at least two different shapes comprising a first shape comprising a first volume and a first luminal diameter that occurs at a first temperature and a second shape comprising a second volume and a second luminal diameter that occurs at a second temperature.
2. The expandable device according to Embodiment 1, wherein the tube assembly is designed in a sealed configuration or an unsealed configuration.
3. The expandable device according to Embodiment 1 or Embodiment 2, wherein when in the unsealed configuration, the tube body of the tube assembly further comprises an overlap region.
4. The expandable device according to Embodiment 3, wherein the overlap region has a length of at least 0.1 mm.
5. The expandable device according to any one of Embodiments 1-4, wherein the tube assembly is longitudinally a straight form, an angled form, a curved form, a L-shaped form, a tee-shaped form, a cross-shaped form, or a reducer form.
6. The expandable device according to any one of Embodiments 1-5, wherein the tube assembly is cylindrical in shape.
7. The expandable device according to any one of Embodiments 1-6, wherein the tube body has a length of between about 2 mm to about 15 mm.
8. The expandable device according to any one of Embodiments 1-7, wherein the expandable device further comprises at least one port.
9. The expandable device according to Embodiment 8, wherein the at least one port further comprises a port cap.
10. The expandable device according to any one of Embodiments 1-9, wherein the expandable device comprises one or more sensors.
11. The expandable device according to Embodiment 10, wherein the one or more sensors monitor one or more physiological and/or structural parameters.
12. The expandable device according to Embodiment 11, wherein the one or more physiological and/or structural parameters comprise a fluid flow rate, a fluid pressure, a fluid viscosity, a fluid contrast, a luminal diameter of the expandable device, a luminal diameter of a vessel, a structural integrity of the expandable device, a structural integrity of the vessel, a label or other tracing component in a fluid, a composition of fluid contained in vessel, or any combination thereof.
13. The expandable device according to any one of Embodiments 10-12, wherein the one or more sensors are located on or integrated with a tube assembly, a tube body, a port, a port cap, a channel of the port, or a lumen of a tube assembly.
14. The expandable device according to any one of Embodiments 1-13, wherein the expandable device further comprises at least one spike.
15. The expandable device according to Embodiment 14, wherein the at least one spike further comprises a spike cap.
16. The expandable device according to any one of Embodiments 1-15, wherein the first shape comprising the first volume and the first luminal diameter occurs at a first temperature that is below the second temperature.
17. The expandable device according to any one of Embodiments 1-16, wherein the first temperature is lower than the second temperature by at least 2° C.
18. The expandable device according to any one of Embodiments 1-17, wherein the first shape comprising the first volume and the first luminal diameter occurs at a first temperature of 25° C. or below.
19. The expandable device according to any one of Embodiments 1-18, wherein the second shape comprising the second volume and the second luminal diameter occurs at a second temperature of 30° C. or above.
20. The expandable device according to any one of Embodiments 1-17, wherein the first shape comprising the first volume and the first luminal diameter occurs at a first temperature of 37° C. or below.
21. The expandable device according to any one of Embodiments 1-17 or Embodiment 20, wherein the second shape comprising the second volume and the second luminal diameter occurs at a second temperature of 41° C. or above.
22. The expandable device according to any one of Embodiments 1-21, wherein the second shape comprises a second volume that is larger than the first volume of the first shape.
23. The expandable device according to any one of Embodiments 1-22, wherein the second shape comprises a second volume that is larger than the first volume of the first shape by at least 5%.
24. The expandable device according to any one of Embodiments 1-23, wherein the second shape comprises a second luminal diameter that is larger than the first luminal diameter of the first shape.
25. The expandable device according to any one of Embodiments 1-24, wherein the second shape comprises a second luminal diameter that is larger than the first luminal diameter of the first shape by at least 5%.
26. The expandable device according to any one of Embodiments 1-25, wherein the first luminal diameter is from about 0.1 mm to about 3.0 mm.
27. The expandable device according to any one of Embodiments 1-26, wherein the second luminal diameter is from about 0.1 mm to about 4.0 mm.
28. The expandable device according to any one of Embodiments 1-27, wherein the expandable metal is a bimetal or a shape memory alloy.
29. The expandable device according to Embodiment 28, wherein the bimetal comprises an outer layer composed of a metal or a metal alloy.
30. The expandable device according to Embodiment 28 or Embodiment 29, wherein the bimetal comprises an inner layer composed of a metal or a metal alloy.
31. The expandable device according to any one of Embodiments 28-30, wherein the metal is composed of a transition metal element, a poor metal element or a metalloid group element.
32. The expandable device according to Embodiment 31, wherein the metal is Aluminum, Antimony, Arsenic, Bismuth, Bohrium, Boron, Cadmium, Chromium, Cobalt, Copernicium, Copper, Dubnium, Gallium, Germanium, Gold, Hafnium, Hassium, Indium, Iridium, Iron, Lead, Manganese, Mercury, Molybdenum, Niobium, Nickel, Osmium, Palladium, Platinum, Polonium, Rethenium, Rhenium, Rhodium, Rutherfordium, Scandium, Seaborgium, Silicon, Silver, Tantaium, Technetium, Tellurium, Thallium, Tin, Titanium, Tungsten, Vanadium, Yttrium, Zinc, or Zirconium.
33. The expandable device according to any one of Embodiments 28-30, wherein the metal alloy is composed of a transition metal element, a poor metal element or a metalloid group element.
34. The expandable device according to Embodiment 33, wherein the metal alloy is composed of two or more of Aluminum, Antimony, Arsenic, Bismuth, Bohrium, Boron, Cadmium, Chromium, Cobalt, Copernicium, Copper, Dubnium, Gallium, Germanium, Gold, Hafnium, Hassium, Indium, Iridium, Iron, Lead, Manganese, Mercury, Molybdenum, Niobium, Nickel, Osmium, Palladium, Platinum, Polonium, Rethenium, Rhenium, Rhodium, Rutherfordium, Scandium, Seaborgium, Silicon, Silver, Tantaium, Technetium, Tellurium, Thallium, Tin, Titanium, Tungsten, Vanadium, Yttrium, Zinc, or Zirconium.
35. The expandable device according to Embodiment 28, wherein the shape memory alloy is composed of a transition metal element, a poor metal element or a metalloid group element.
36. The expandable device according to Embodiment 35, wherein the shape memory alloy is composed of two or more of Aluminum, Antimony, Arsenic, Bismuth, Bohrium, Boron, Cadmium, Chromium, Cobalt, Copernicium, Copper, Dubnium, Gallium, Germanium, Gold, Hafnium, Hassium, Indium, Iridium, Iron, Lead, Manganese, Mercury, Molybdenum, Niobium, Nickel, Osmium, Palladium, Platinum, Polonium, Rethenium, Rhenium, Rhodium, Rutherfordium, Scandium, Seaborgium, Silicon, Silver, Tantaium, Technetium, Tellurium, Thallium, Tin, Titanium, Tungsten, Vanadium, Yttrium, Zinc, or Zirconium.
37. The expandable device according to Embodiment 36, wherein the shape memory alloy is Cobolt-Nichol-Aluminum (CoNiAl), Cobolt-Nichol-Gallium (CoNiGa), Copper-Tin (CuSn), Copper-Zinc (CuZn), Copper-Aluminum-Iron (CuAlFe), Copper-Aluminum-Nickel (CuAlNi), Copper-Zinc-Aluminum (CuZnAl), Copper-Zinc-Silicon (CuZnSi), Copper-Zinc-Tin (CuZnSn), Gold-Cadmium (AuCd), Hafnium-Titanium-Nickel (HfTiNi), Iron-Platinum (FePt), Iron-Manganese-Silicon (FeMnSi), Iron-Zinc-Copper-Aluminum (FeZnCuAl), Manganese-Copper (MnCu), Nickel-Iron-Gallium (NiFeGa), Nickel-Manganese-Gallium (NiMnGa), Nickel-Titanium (NiTi), Nickel-Titanium-Copper (NiTiCu), Nickel-Zirconium-Titanium (NiZrTi), Nickel-Iron-Zinc-Aluminum (NiFeZnAl), Silver-Cadmium (AgCd), Titanium-Palladium-Nickel (TiPdNi), Titanium-Niobium (TiNb), Titanium-Niobium-Aluminum (TiNbAl), Uranium-Niobium (Nb), or Zirconium-Copper-Zinc (ZrCuZn).
38. A method of employing an expandable device as defined in any one of Embodiments 1-37, the method comprising the steps of: a) inserting one end of the expandable device into a first lumen of a first vessel; b) inserting the other end of the expandable device into a second lumen of a second vessel; and c) enlarging the expandable device.
39. The method according to Embodiment 38, wherein the enlarging occurs by: 1) allowing the device to be warmed by the body temperature of an individual emanating from the surrounding tissue; 2) injecting a fluid into the expandable device that is sufficiently warm enough to enlarge the device; or 3) using a heating device that warms the expandable device to a temperature sufficiently warm enough to enlarge the device, or any combination thereof.
40. A method of employing an expandable device as defined in any one of Embodiments 1-37, the method comprising the steps of: a) inserting an end of a first lumen of a first vessel into a first end of the expandable device; b) inserting an end of a second lumen of a second vessel into an opposite second end of the expandable device; and c) contracting the expandable device.
41. The method according to Embodiment 40, wherein the contracting occurs by allowing the device to be cooled.
42. The method according to Embodiment 40 or Embodiment 41, further comprising the step of enlarging the expandable device prior to the steps of inserting an end of a first lumen of a first vessel into a first end of the expandable device or inserting an end of a second lumen of a second vessel into an opposite second end of the expandable device.
43. The method according to Embodiment 42, wherein the enlarging occurs by 1) injecting a fluid into the expandable device that is sufficiently warm enough to enlarge the device; or 2) using a heating device that warms the expandable device to a temperature sufficiently warm enough to enlarge the device, or any combination thereof.
44. The method according to any one of Embodiments 38-43, further comprising the step of applying suction to the inserted expandable device.
45. The method according to any one of Embodiments 38-44, further comprising the step of attaching a fluid-filled device to a port on the expandable device.
46. The method according to any one of Embodiments 38-45, further comprising the step of placing a portion of the first vessel comprising tunia externa over a port on the expandable device in order to prevent retraction of the first vessel, or placing a portion of the second vessel comprising tunia externa over a port on the expandable device in order to prevent retraction of the second vessel, or both.
47. The method according to any one of Embodiments 38-46, wherein the first vessel is a circulatory vessel, a neuronal vessel, a gastrointestinal vessel, an urogenital vessel, an endocrine vessel, a respiratory vessel.
48. The method according to Embodiment 47, wherein the circulatory vessel is a blood vessel or a lymphatic vessel.
49. The method according to Embodiment 48, wherein the blood vessel is an artery, an arteriole, a vein, or a veiniole.
50. The method according to Embodiment 48, wherein the lymphatic vessel is a lymph vessel or a lymph node.
51. The method according to Embodiment 47, wherein the gastrointestinal vessel is a small intestine, a large intestine, or a bile duct
52. The method according to Embodiment 51, wherein the small intestine is a duodenum, a jujenum, or an illium.
53. The method according to Embodiment 53, wherein the large intestine is a colon or a rectum.
54. The method according to Embodiment 47, wherein the urogenital vessel is a ureter, a urethra, a fallopian tube, or a vas deference vessel.
55. The method according to Embodiment 47, wherein the endocrine vessel is a pancreatic duct, a thyroid gland duct, an adrenal duct, or a pituitary duct.
56. The method according to Embodiment 47, wherein the respiratory vessel is a bronchus or a bronchiole.
57. The method according to any one of Embodiments 38-56, wherein the second vessel is a circulatory vessel, a neuronal vessel, a gastrointestinal vessel, an urogenital vessel, an endocrine vessel, a respiratory vessel.
58. The method according to Embodiment 57, wherein the circulatory vessel is a blood vessel or a lymphatic vessel.
59. The method according to Embodiment 58, wherein the blood vessel is an artery, an arteriole, a vein, or a veiniole.
60. The method according to Embodiment 58, wherein the lymphatic vessel is a lymph vessel or a lymph node.
61. The method according to Embodiment 57, wherein the gastrointestinal vessel is a small intestine, a large intestine, or a bile duct.
62. The method according to Embodiment 61, wherein the small intestine is a duodenum, a jujenum, or an illium.
63. The method according to Embodiment 61, wherein the large intestine is a colon or a rectum.
64. The method according to Embodiment 57, wherein the urogenital vessel is a ureter, a urethra, a fallopian tube, or a vas deference vessel.
65. The method according to Embodiment 57, wherein the endocrine vessel is a pancreatic duct, a thyroid gland duct, an adrenal duct, or a pituitary duct.
66. The method according to Embodiment 57, wherein the respiratory vessel is a bronchus or a bronchiole.
67. The method according to any one of Embodiments 38-66, wherein the method creates an end-to-end anastomosis, an end-to-side anastomosis, or a side-to-side anastomosis.
68. The method according to any one of Embodiments 38-67, wherein the method creates an arterio-arterial anastomosis, a veno-venous anastomosis, a lympho-lymphatic anastomosis, an arterio-venous anastomosis, a lympho-venous anastomosis, a lymph node to a vein anastomosis, a gastrointestinal anastomosis, an urogenital anastomosis, an endocrine anastomosis, or a respiratory anastomosis.
69. A kit comprising one or more expandable devices as defined in any one of Embodiments 1-37.
70. The kit according to Embodiment 69, further comprising one or more containers comprising a physiological-acceptable fluid used to enlarge an expandable device.
71. The kit according to Embodiment 69 or Embodiment 70, further comprising instructional material providing instructions on how to use an expandable device contained therein.
72. The kit according to Embodiment 71, wherein the instructional material provides instructions on how to perform a method as defined in any one of Embodiments 38-68.
73. The kit according to any one of Embodiments 69-72, further comprising a heating device useful for warming the expandable device in order to enlarge the expandable device.
74. Use of an expandable devices as defined in any one of Embodiments 1-37 to create a connection between two or more tubular structures.
75. The use according to Embodiment 74, wherein the use comprises a method as defined in any one of Embodiments 38-68.

### EXAMPLES

The following non-limiting examples are provided for illustrative purposes only in order to facilitate a more complete understanding of the disclosed subject matter. These examples should not be construed to limit any of the embodiments described in the present specification, including those pertaining to an expandable device, kits comprising an expandable device, and/or methods and uses for forming an anastomosis. Specifically, while the examples presented involve embodiments not having spikes or the like, it will be appreciated that in certain clinical contexts such will be indicated for use, with the device according to aspects of the present invention yet providing other benefits in use.

### Example 1

### Head and Neck Reconstructive Surgery

This example demonstrates the use of an expandable device disclosed herein to create arterio-arterial and veno-venous anastomoses during a head and neck reconstructive surgery. A surgeon is performing head and neck reconstructive surgery on a 48 year old female. The surgeon removes a radial forearm free flap, which is a unit of tissue supplied by artery(ies) and drained by the vein(s) and typically includes skin, subcutaneous tissue, muscle, and sometimes bone. Alternatively, the surgeon could use a fibula free flap, an anterolateral thigh flap, an iliac crest flap, and/or a rectus abdominus flap. Small vessels measuring 1-3 mm in diameter are connected under high power surgical microscope illumination using an expandable device disclosed herein. The surgeon inserts one end of the expandable device into the lumen of an artery present in the face and the other end of the expandable device into the lumen of an artery present in the flap. The surgeon ensures that the vessel ends slide up against a port and are properly positioned before attaching a syringe filled with warm saline to the port. The surgeon then injects the warm saline into the expandable device. Both the body temperature of the woman and the temperature of the warm saline cause the expandable device to enlarge and fit tightly against the internal walls of the vessels. The surgeon then removes the syringe. In an alternative example, an expandable device without a port is used and enlargement occurs simply due to the body temperature of the woman and/or through the use of a heating device that radiates warm air. This process is repeated for any other arterio-arterial and/or veno-venous anastomoses the surgeon wishes to create in order to ensure proper blood supply of the tissue flap. This example demonstrates that arterio-arterial and veno-venous anastomoses can be created using an expandable device disclosed herein without the use of sutures, barbs, or spikes, and thus, without any trauma to the endothelium.

### Example 2

### Breast Reconstructive Surgery

This example demonstrates the use of an expandable device disclosed herein to create arterio-arterial and veno-venous anastomoses during a breast reconstructive surgery. A surgeon is performing breast reconstructive surgery on a 53 year old female who had previously undergone a mastectomy. The surgeon removes an abdomen flap supplied by artery(ies) and drained by the vein(s). Alternatively, the surgeon could use a flap of tissue from a buttock, hip, lateral thigh, or medial thigh. Small vessels measuring 2-3 mm in diameter are connected under high power surgical microscope illumination using an expandable device disclosed herein. The surgeon inserts a vein present in the chest wall into one end of the expandable device and a vein present in the flap into the other end of the expandable device. The surgeon ensures that the vessel ends slide up against a port and are properly positioned before attaching a syringe filled with room temperature saline to the port. The surgeon then injects the saline into the expandable device. Both the body temperature of the woman and the temperature of the saline cause the expandable device to contract and fit tightly against the external walls of the vessels. The surgeon then removes the syringe. In an alternative example, an expandable device without a port is used and contraction occurs simply due to the body temperature of the woman and/or through the use of a cooling device that radiates cool air. Alternatively, the expandable device may be heated to a temperature above the body temperature of the woman before use of the device, as by use of a heating device that radiates warm air or an elevated temperature saline bath, for example, so as to first expand the device in order to accommodate insertion of the first and second vessels; upon then inserting the device into the relatively lower temperature environment of the patient's body, the device then contracts to its unexpanded configuration so as to tighten onto the vessels. This process is repeated for any other arterio-arterial and/or veno-venous anastomoses on branches of the subclavian artery and vein, such as the internal mammary vessels, or branches of the axillary artery and vein, such as the thoracodorsal vessels the surgeon wishes to create in order to ensure proper blood supply of the tissue flap. This example demonstrates that arterio-arterial and veno-venous anastomoses can be created using an expandable device disclosed herein without the use of sutures, barbs, or spikes, and thus, without any trauma to the endothelium.

### Example 3

### Limb Surgery

This example demonstrates the use of an expandable device disclosed herein to create arterio-arterial and veno-venous anastomoses during a limb surgery, such as, e.g., a lower extremity reconstruction for limb salvage in trauma, a diabetic ulcer reconstruction, or an ischemic wound. A surgeon is performing a surgery on the lower leg of a 62 year old male in order to repair damage due to a diabetic ulcer. The surgeon removes a rectus abdominus flap supplied by artery(ies) and drained by the vein(s). Alternatively, the surgeon could use an anteriolateral thigh flap or a lattisimusdorsi flap. Small vessels measuring 1-2 mm in diameter are connected under high power surgical microscope illumination using an expandable device disclosed herein. The surgeon inserts one end of the expandable device into the lumen of a vein present in the lower leg and the other end of the expandable device into the lumen of a vein present in the flap. The surgeon ensures that the vessel ends slide up against a port and are properly positioned before attaching a syringe filled with warm saline to the port. The surgeon then injects the warm saline into the expandable device. Both the body temperature of the man and the temperature of the warm saline cause the expandable device to enlarge and fit tightly against the internal walls of the vessels. The surgeon then removes the syringe. In an alternative example, an expandable device without a port is used and enlargement occurs simply due to the body temperature of the woman and/or through the use of a heating device that radiates warm air. This process is repeated for any other arterio-arterial and/or veno-venous anastomoses the surgeon wishes to create in order to ensure proper blood supply of the tissue flap. This example demonstrates that arterio-arterial and veno-venous anastomoses can be created using an expandable device disclosed herein without the use of sutures, barbs, or spikes, and thus, without any trauma to the endothelium.

### Example 4

### Digit Reimplantation Surgery

This example demonstrates the use of an expandable device disclosed herein to create arterio-arterial and veno-venous anastomoses during a reimplantation surgery of a severed digit or wrist laceration. A surgeon is performing a surgery to reattach a thumb of a 28 year old male cut off in a power saw accident. Small vessels measuring 0.5-1 mm in diameter are connected under high power surgical microscope illumination using an expandable device disclosed herein. The surgeon inserts one end of the expandable device into the lumen of an artery present in the amputated thumb and the other end of the expandable device into the lumen of an artery present in the thumb stump on the hand. The surgeon ensures that the vessel ends slide up against a port and are properly positioned before attaching a syringe filled with warm saline to the port. The surgeon then injects the warm saline into the expandable device. Both the body temperature of the man and the temperature of the warm saline cause the expandable device to enlarge and fit tightly against the internal walls of the vessels. The surgeon then removes the syringe. In an alternative example, an expandable device without a port is used and enlargement occurs simply due to the body temperature of the man and/or through the use of a heating device that radiates warm air. This process is repeated for any other arterio-arterial and/or veno-venous anastomoses the surgeon wishes to create in order to ensure proper blood supply to the reimplanted thumb. This example demonstrates that arterio-arterial and veno-venous anastomoses can be created using an expandable device disclosed herein without the use of sutures, barbs, or spikes, and thus, without any trauma to the endothelium.

### Example 5

### Neurosurgical Revascularization Surgery of Brain

This example demonstrates the use of an expandable device disclosed herein to create arterio-arterial and veno-venous anastomoses during a neurosurgical revascularization surgery of the brain. A surgeon is performing an extracrainial-intracrainial bypass surgery on a 69 year old male who was presenting with an occlusion of the Circle of Willis that is obstructing blood flow to the middle cerebral artery. Small vessels measuring 1-2 mm in diameter are connected under high power surgical microscope illumination using an expandable device disclosed herein. The surgeon inserts an artery present proximal to the occlusion into one end of the expandable device and an artery present distal to the occlusion into the other end of the expandable device. The surgeon ensures that the vessel ends slide up against a port and are properly positioned before attaching a syringe filled with room temperature saline to the port. The surgeon then injects the saline into the expandable device. Both the body temperature of the man and the temperature of the saline cause the expandable device to contract and fit tightly against the external walls of the vessels. The surgeon then removes the syringe. In an alternative example, an expandable device without a port is used and contraction occurs simply due to the body temperature of the man and/or through the use of a cooling device that radiates cool air. Alternatively, the expandable device may be heated to a temperature above the body temperature of the man before use of the device, as by use of a heating device that radiates warm air or an elevated temperature saline bath, for example, so as to first expand the device in order to accommodate insertion of the first and second vessels; upon then inserting the device into the relatively lower temperature environment of the patient's body, the device then contracts to its unexpanded configuration so as to tighten onto the vessels. This process is repeated for any other arterio-arterial and/or veno-venous anastomoses needed to connect branches of superficial temporal vessels to the middle cerebral vessels, distal to the occlusion, that the surgeon wishes to create in order to ensure proper blood supply to the brain. This example demonstrates that arterio-arterial and veno-venous anastomoses can be created using an expandable device disclosed herein without the use of sutures, barbs, or spikes, and thus, without any trauma to the endothelium.

### Example 6

### Surgery to Create an Arteriovenous Fistula

This example demonstrates the use of an expandable device disclosed herein to create arterio-venous anastomoses. A surgeon is performing a surgery to create an arteriovenous fistula in the left arm of a 43 year old male who undergoes renal dialysis to treat his kidney disease. Small vessels measuring 2-3 mm in diameter are connected under high power surgical microscope illumination using an expandable device disclosed herein. The surgeon inserts one end of the expandable device into the lumen of an artery present in the lower left arm and the other end of the expandable device into the lumen of a vein present in the lower left arm. The surgeon ensures that the vessel ends slide up against a port and are properly positioned before attaching a syringe filled with warm saline to the port. The surgeon then injects the warm saline into the expandable device. Both the body temperature of the man and the temperature of the warm saline cause the expandable device to enlarge and fit tightly against the internal walls of the vessels. The surgeon then removes the syringe. In an alternative example, an expandable device without a port is used and enlargement occurs simply due to the body temperature of the man and/or through the use of a heating device that radiates warm air. This example demonstrates that arteriovenous anastomoses can be created using an expandable device disclosed herein without the use of sutures, barbs, or spikes, and thus, without any trauma to the endothelium.

### Example 7

### Lower Limb Surgery

This example demonstrates the use of an expandable device disclosed herein to create arterio-arterial and veno-venous anastomoses during a lower limb surgery to restore vascularization due to, e.g., a vascular disease or diabetes. A surgeon is performing a femoral-tibial bypass on lower right leg of a 74 year old male in order to revascularize the limb. Small vessels measuring 2-3 mm in diameter are connected under high power surgical microscope illumination using an expandable device disclosed herein. The surgeon inserts an artery present proximal to the damaged tissue into one end of the expandable device and an artery present distal to the damaged tissue into the other end of the expandable device. The surgeon ensures that the vessel ends slide up against a port and are properly positioned before attaching a syringe filled with room temperature saline to the port. The surgeon then injects the saline into the expandable device. Both the body temperature of the man and the temperature of the saline cause the expandable device to contract and fit tightly against the external walls of the vessels. The surgeon then removes the syringe. In an alternative example, an expandable device without a port is used and contraction occurs simply due to the body temperature of the man and/or through the use of a cooling device that radiates cool air. Alternatively, the expandable device may be heated to a temperature above the body temperature of the man before use of the device, as by use of a heating device that radiates warm air or an elevated temperature saline bath, for example, so as to first expand the device in order to accommodate insertion of the first and second vessels; upon then inserting the device into the relatively lower temperature environment of the patient's body, the device then contracts to its unexpanded configuration so as to tighten onto the vessels. This process is repeated for any other arterio-arterial and/or veno-venous anastomoses the surgeon wishes to create in order to ensure proper blood supply of the lower right leg. This example demonstrates that arterio-arterial and veno-venous anastomoses can be created using an expandable device disclosed herein without the use of sutures, barbs, or spikes, and thus, without any trauma to the endothelium.

### Example 8

### Lympho-Lymphatic Anastomoses

This example demonstrates the use of an expandable device disclosed herein to create lympho-lymphatic anastomoses and/or lympho-venous anastomoses to restore lymphatic circulation. A surgeon is performing surgery on a 61 year old female to restore lymphatic circulation to her left leg due to a lymphatic obstruction. The surgeon removes an omental flap containing a rich plexus of intact lymphatics. Alternatively, the surgeon could use a flap of tissue containing a rich plexus of intact lymphatics. Small vessels measuring 0.1-0.5 mm in diameter are connected under high power surgical microscope illumination using an expandable device disclosed herein. The surgeon inserts one end of the expandable device into the lumen of a vein present in the lower leg and the other end of the expandable device into the lumen of a lymph vessel present in the flap. The surgeon ensures that the vessel ends slide up against a port and are properly positioned before attaching a syringe filled with warm saline to the port. The surgeon then injects the warm saline into the expandable device. Both the body temperature of the woman and the temperature of the warm saline cause the expandable device to enlarge and fit tightly against the internal walls of the vessels. The surgeon then removes the syringe. In an alternative example, an expandable device without a port is used and enlargement occurs simply due to the body temperature of the woman and/or through the use of a heating device that radiates warm air. This process is repeated for any other lympho-lymphatic anastomosis and/or lympho-venous anastomoses the surgeon wishes to create in order to ensure proper restoration of lymphatic circulation. Additionally, lymph node transplants can also be done from an unaffected extremity and a lymph node-to-venous anastomosis done restore lymphatic outflow. This example demonstrates that lympho-lymphatic and/or lympho-venous and/or lymph node to vein anastomoses can be created using an expandable device disclosed herein without the use of sutures, barbs, or spikes, and thus, without any trauma to the endothelium.

### Example 9

### Surgery to Create Urogenital Anastomoses

This example demonstrates the use of an expandable device disclosed herein to create urogenital anastomoses. A surgeon is performing a surgery to create a urogenital anastomsis in the right ureter of a 49 year old male diagnosed with a kidney stone that he has been unable to pass using standard treatments. The surgeon cuts the ureter before and after the blockage to remove the section containing the stone. The ureter ends, measuring 3-5 mm in diameter, are connected under high power surgical microscope illumination using an expandable device disclosed herein. The surgeon inserts one end of the expandable device into the lumen of the ureter end before the blockage and the other end of the expandable device into the lumen of the ureter end after the blockage. The surgeon ensures that the ureter ends slide up against a port and are properly positioned before attaching a syringe filled with warm saline to the port. The surgeon then injects the warm saline into the expandable device. Both the body temperature of the man and the temperature of the warm saline cause the expandable device to enlarge and fit tightly against the internal walls of the rejoined ureter vessels. The surgeon then removes the syringe. In an alternative example, an expandable device without a port is used and enlargement occurs simply due to the body temperature of the man and/or through the use of a heating device that radiates warm air. This example demonstrates that urogenital anastomoses can be created using an expandable device disclosed herein without the use of sutures, barbs, or spikes, and thus, without any trauma to the endothelium.

### Example 10

### Surgery to Create Urogenital Anastomoses

This example demonstrates the use of an expandable device disclosed herein to create urogenital anastomoses. A surgeon is performing a surgery to create a urogenital anastomsis in the right ureter of a 49 year old male who previously has his vas deferens cut in a sterility procedure. The surgeon cuts the vas deferens remnants. The ends, measuring 2-3 mm in diameter, are connected under high power surgical microscope illumination using an expandable device disclosed herein. The surgeon inserts one end of the expandable device into the lumen of one vas deferens remnant and the other end of the expandable device into the lumen of the other vas deferens remnant. The surgeon ensures that the remnant ends slide up against a port and are properly positioned before attaching a syringe filled with warm saline to the port. The surgeon then injects the warm saline into the expandable device. Both the body temperature of the man and the temperature of the warm saline cause the expandable device to enlarge and fit tightly against the internal walls of the rejoined vas deferens vessels. The surgeon then removes the syringe. In an alternative example, an expandable device without a port is used and enlargement occurs simply due to the body temperature of the man and/or through the use of a heating device that radiates warm air. This process is repeated for any other urogenital anastomoses that the surgeon wishes to create in order to ensure proper fertility. This example demonstrates that urogenital anastomoses can be created using an expandable device disclosed herein without the use of sutures, barbs, or spikes, and thus, without any trauma to the endothelium.

### Example 11

### Surgery to Create Endocrine Anastomoses

This example demonstrates the use of an expandable device disclosed herein to create endocrine anastomoses. A surgeon is performing a surgery to create an endocrine anastomsis in an infant born with a defective pancreatic duct creating a blockage. The surgeon cuts the pancreatic duct before and after the blockage to remove the section containing the defective duct. The pancreatic duct ends, measuring 0.5-1 mm in diameter are connected under high power surgical microscope illumination using an expandable device disclosed herein. The surgeon inserts one end of the expandable device into the lumen of the pancreatic duct end before the blockage and the other end of the expandable device into the lumen of the pancreatic duct end after the blockage. The surgeon ensures that the duct ends slide up against a port and are properly positioned before attaching a syringe filled with warm saline to the port. The surgeon then injects the warm saline into the expandable device. Both the body temperature of the infant and the temperature of the warm saline cause the expandable device to enlarge and fit tightly against the internal walls of the rejoined pancreatic duct vessels. The surgeon then removes the syringe. In an alternative example, an expandable device without a port is used and enlargement occurs simply due to the body temperature of the infant and/or through the use of a heating device that radiates warm air. This process is repeated for any other endocrine anastomoses that the surgeon wishes to create in order to ensure proper pancreatic function. This example demonstrates that urogenital anastomoses can be created using an expandable device disclosed herein without the use of sutures, barbs, or spikes, and thus, without any trauma to the endothelium.

### Example 12

### Surgery to Create Respiratory Anastomoses

This example demonstrates the use of an expandable device disclosed herein to create respiratory anastomoses. A surgeon is performing a surgery to create a respiratory anastomsis in a 29 year old woman who was injured in an automobile accident which caused lacerations in her left lung. Bronchiole measuring 6-8 mm in diameter are connected under high power surgical microscope illumination using an expandable device disclosed herein. The surgeon inserts one end of the expandable device into the lumen of one end of a severed bronchiole and the other end of the expandable device into the lumen of the other end of a severed bronchiole. The surgeon ensures that the vessel ends slide up against a port and are properly positioned. The body temperature of the woman causes the expandable device to enlarge and fit tightly against the internal walls of the rejoined bronchiole. In an alternative example, an expandable device is enlarged through the use of a heating device that radiates warm air. This process is repeated for any other respiratory anastomoses that the surgeon wishes to create in order to ensure proper air supply to the lungs. This example demonstrates that respiratory anastomoses can be created using an expandable device disclosed herein without the use of sutures, barbs, or spikes, and thus, without any trauma to the endothelium.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An expandable device comprising a tube assembly composed of an expandable metal, the tube assembly comprising:
a tube body having a lumen defining a first luminal opening and a second luminal opening,
wherein the tube body forms the lumen and the first and second luminal openings are at each end of the tube assembly, and
wherein the expandable device is configured to exist in at least two different shapes each comprising a volume, the at least two different shapes comprising a first shape comprising a first volume and a first luminal diameter that occurs at a first temperature and a second shape comprising a second volume and a second luminal diameter that occurs at a second temperature.

2. The expandable device according to claim 1, wherein the tube body of the tube assembly further comprises an overlap region.

3. The expandable device according to claim 1, wherein the expandable device further comprises at least one port in fluid communication with the lumen.

4. The expandable device according to claim 1, wherein the expandable device comprises one or more sensors.

5. The expandable device according to claim 4, wherein the one or more sensors monitor one or more physiological and/or structural parameters.

6. The expandable device according to claim 5, wherein the one or more physiological and/or structural parameters comprise a fluid flow rate, a fluid pressure, a fluid viscosity, a fluid contrast, a luminal diameter of the expandable device, a luminal diameter of a vessel, a structural integrity of the expandable device, a structural integrity of the vessel, a label or other tracing component in a fluid, a composition of fluid contained in vessel, or any combination thereof.

7. The expandable device according to claim 4, wherein the one or more sensors are located on or integrated with the tube assembly, the tube body, a port, a port cap, a channel of the port, or the lumen of a tube assembly.

8. The expandable device according to claim 1, wherein the expandable device further comprises at least one spike.

9. The expandable device according to claim 1, wherein the first shape comprising the first volume and the first luminal diameter occurs at a first temperature that is below the second temperature.

10. The expandable device according to claim 9, wherein the first shape comprising the first volume and the first luminal diameter occurs at a first temperature of 25° C. or below.

11. The expandable device according to claim 10, wherein the second shape comprising the second volume and the second luminal diameter occurs at a second temperature of 30° C. or above.

12. The expandable device according to claim 9, wherein the first shape comprising the first volume and the first luminal diameter occurs at a first temperature of 37° C. or below.

13. The expandable device according to claim 12, wherein the second shape comprising the second volume and the second luminal diameter occurs at a second temperature of 41° C. or above.

14. The expandable device according to claim 1, wherein the expandable metal is a bimetal or a shape memory alloy.

15. A method of employing an expandable device as defined in claim 1, the method comprising the steps of:
a) inserting one end of the expandable device into a first lumen of a first vessel;
b) inserting the other end of the expandable device into a second lumen of a second vessel; and
c) enlarging the expandable device.

16. The method according to claim 15, wherein the enlarging occurs by: 1) allowing the device to be warmed by the body temperature of an individual emanating from the surrounding tissue; 2) injecting a fluid into the expandable device that is sufficiently warm enough to enlarge the device; or 3) using a heating device that warms the expandable device to a temperature sufficiently warm enough to enlarge the device, or any combination thereof.

17. A method of employing an expandable device as defined in claim 1, the method comprising the steps of:
a) inserting an end of a first lumen of a first vessel into a first end of the expandable device;
b) inserting an end of a second lumen of a second vessel into an opposite second end of the expandable device; and
c) contracting the expandable device.

18. The method according to 17, further comprising the step of enlarging the expandable device prior to the steps of inserting an end of a first lumen of a first vessel into a first end of the expandable device or inserting an end of a second lumen of a second vessel into an opposite second end of the expandable device.

19. An expandable device comprising a tube assembly composed of an expandable metal, the tube assembly comprising:
a tube body having a lumen defining a first luminal opening and a second luminal opening,
wherein the tube body forms the lumen and the first and second luminal openings are at each end of the tube assembly; and
a port in fluid communication with the lumen,
wherein the expandable device is configured to exist in at least two different shapes each comprising a volume, the at least two different shapes comprising a first shape comprising a first volume and a first luminal diameter that occurs at a first temperature and a second shape comprising a second volume and a second luminal diameter that occurs at a second temperature.

20. An expandable device comprising a tube assembly composed of an expandable metal, the tube assembly comprising:
a tube body having a lumen defining a first luminal opening and a second luminal opening,
wherein the tube body forms the lumen and the first and second luminal openings are at each end of the tube assembly;
at least one spike formed on the tube body so as to extend substantially radially therefrom;
at least one port in fluid communication with the lumen; and
at least one sensor located on or integrated with the tube assembly, the tube body, the port, a port cap, a channel of the port, or the lumen of the tube assembly,
wherein the expandable device is configured to exist in at least two different shapes each comprising a volume, the at least two different shapes comprising a first shape comprising a first volume and a first luminal diameter that occurs at a first temperature and a second shape comprising a second volume and a second luminal diameter that occurs at a second temperature.
